(19) United States
(12) Patent Application Publication
(10) Pub. No.: US 2005/0130910 A1
(43) Pub. Date: Jun. 16, 2005

(54) Immobilized N-substituted tricyclic 3-aminopyrazoles for the identification of biomolecular targets

(76) Inventors: Chih Y. Ho, Lansdale, PA (US); Robert A. Galemmo JR., Paoli, PA (US); Dana L. Johnson, Upper Black Eddy, PA (US); Jay M. Mei, North Wales, PA (US); Stanley M. Belkowski, Norristown, PA (US); Richard William Connors, Harleysville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/986,500

(22) Filed: Nov. 10, 2004

Related U.S. Application Data
(63) Continuation-in-part of application No. 10/438,152, filed on May 14, 2003.
(60) Provisional application No. 60/380,735, filed on May 15, 2002.
(60) Provisional application No. 60/519,875, filed on Nov. 13, 2003.

(51) Int. Cl. A61K 31/7056; A61K 31/427; A61K 31/422; A61K 31/4162; C07H 17/00; C07D 417/02; C07D 413/02

(52) U.S. Cl. 514/23; 514/365; 514/374; 514/406; 536/17.4; 548/181; 548/215

(57) ABSTRACT

The present invention relates to immobilized N-substituted tricyclic 3-aminopyrazole compounds of Formula 1 as tools for the identification of bimolecular targets in cells of therapeutic significance, profiling the selectivity of compounds, prediction of possible related toxicities and exploration of mechanisms of action in biological systems for therapeutic indications related to compounds. These agents can be used to identify biomolecules with the potential to interact with the immobilized reagent. The identified biomolecule may be then be used as a therapeutic target, serve as a marker of drug action, or alternatively describe an untoward or toxic potential of the immobilized agent.

### FIELD OF THE INVENTION

The current invention relates to the synthesis and biological application of immobilized N-substituted tricyclic 3-aminopyrazole compounds as tools for the identification of bimolecular targets in cells of therapeutic significance, profiling the selectivity of compounds, prediction of possible related toxicities and exploration of mechanisms of action in biological systems for therapeutic indications related to compounds.

### BACKGROUND OF THE INVENTION

Characterization of the molecular/cellular targets of a compound is the first step in understanding how the compound may work in a biological system and determine potential therapeutic indications.

To date, applications of immobilized compounds as investigative research tools include the following: immobilized anti-apoptotic compound CGP 3466 for target identification (Zimmermann, et, al. Bioorganic & Medicinal Chemistry Letters 8, 1998, 1195-1200); intracellular target identification of immobilized purine CDK inhibitors using an agarose matrix (Knockaert, et al. Chemistry & Biology 2000, vol 7 No 6, 411-422); use of immobilized Paullones using an agarose matrix for the identification of malate dehydrogenase as cellular target underlying anit-proliferative activity (Knockaert, et al. JBC 2002, vol 277, No 28, 25493-25501); synthesis and binding of biotinylated glucose derivatives which probe glucose transporter proteins in erythrocytes (Hashimoto, et al., Carbohydrate Research, 2001, 331(2), 119-127); use of a magnetic particle as the binding mediator use of magnetic separation techniques for cells and biological molecules (Applications of magnetic nanoparticles in biomedicine. Pankhurst, et al., J. of Physics D: Applied Physics, 2003, 36(13), R167-R181). See also: Knockaert et al. (Intracellular Targets of Paullones, J. Biol. Chem. 2002, 277(28) 25493-25501) (disclosing agarose matrix immobilized paullone compounds, gwennpaullone); (Schnier et al. Identification of cytosolic aldehyde dehydrogenase-1 from non-small cell lung carcinomas as a flavopirirdol-binding protein, FEBS Letters 1999, 454, 100-104) (demonstrating the interaction of the well-know CDK inhibitor, flavopiridol, with another protein not in the CDK family by an immobilization experiment; proteins isolated and identified included cytosolic aldehyde dehydrogenase-1 (ALDH-1)).

Additional examples in the literature include, those presented in Immobilized Biomolecules in Analysis (1998), 15-34. Editor(s): Cass, Tony; Ligler, Frances S. Publisher: Oxford University Press, Oxford, UK CODEN: 67NBAN Conference; General Review written in English. CAN 131:41599 AN 1999:214232, in which virtually any biologically active compounds including antibodies, receptors, enzymes, inhibitors, hormones, nucleic acids, drugs, and toxins are biotinylated, bound to an avidin surface, and the surface then used for a variety of isolation purposes, including the discovery of target molecules that can be isolated via interaction with the immobilized biotinylated molecule (the binder). Also disclosed is use of the immobilized avidin as a simple capture system: to capture the biotinylated binder in complex with its partner (the target) for isolation.

U.S. patent application Ser. No. 10/438,152, “filed May 14, 2003, and PCT/US/03/15193, filed May 13, 2003, “N-SUBSTITUTED TRICYCLIC 3-AMINOPYRAZOLES AS INHIBITORS FOR THE TREATMENT OF CELL PROLIFERATIVE DISORDERS,” both of which are incorporated herein in their entirety, disclose a novel series of N-substituted tricyclic 3-aminopyrazoles compounds which possess direct anti-proliferative activities against human tumor cells in addition to demonstrated inhibitory activity against platelet-derived growth factor (PDGF) receptor tyrosine kinase (RTK).

Anti-angiogenic therapy appears as an attractive novel way of interfering with tumor growth. Indeed, numerous studies in animal models have demonstrated striking effects in inhibiting tumor growth by targeting angiogenic growth factors such as vascular endothelial growth factor (VEGF), acidic and basic fibroblast growth factor (aFGF, bFGF) and PDGF. The receptors for VEGF and PDGF belong to a specific super family of RTK (receptor tyrosine kinases). Therefore, in addition to their roles in treating other cell proliferative disorders, clinically useful PDGF-RTK inhibitors are highly warranted for anti-angiogenic therapy as well as for direct inhibition of certain tumor types.

Small molecule inhibitors of the receptor tyrosine kinase constitute a novel class of drugs with large potential (Druker and Lydon, J. Clin. Invest., 105:3-7, 2000, and references therein). Since 1995, a number of small molecule inhibitors for PDGF receptor autophosphorylation have been characterized. Some examples are listed below.

JP 06087834 (Zimmermann) discloses N-phenyl-2-pyrimidine-amine derivatives which have tumor inhibitory activity and are useful for treating tumors in warm-blooded animals including human beings. Derivatives of this group of compounds, compound CGP53716 (Buchdunger et al., PNAS, 92:2558-2562,1995) and compound STI-571 (Buchdunger et al., Cancer Res, 56:100-4, 1996), have been shown to inhibit PDGF-R autophosphorylation.

JP 11158149 (Kubo et al.) discloses quinoline derivatives for the treatment of diseases such as tumors and diabetic retinopathy. Derivatives of this group of compounds, compound Ki6783 (Yagi et al., Exp. Cell Res. 243:285-292, 1997) and compound Ki6896 (Yagi et al., Gen. Pharmacol. 31:765-773,1998), have been shown to inhibit PDGF-R autophosphorylation.

U.S. Pat. No. 5,932,580 (Levitzki et al.) discloses PDGF receptor kinase inhibitory compounds of the quinoxaline family including Tyrphostin, ATP-competitive inhibitors of the receptor kinase.

U.S. Pat. No. 5,409,930 (Spada, et al.) discloses bis mono- and/or bicyclic aryl and/or heteroaryl compounds exhibiting protein tyrosine kinase inhibition activity. Compound RPR01511A, a derivative of this group of compound, has been shown to inhibit PDGF-R autophosphorylation (Bilder et al., Circulation. 99(25):3292-9, 1999).

U.S. Pat. No. 5,563,173 (Yatsu, et al.) discloses a method of inhibiting the proliferation of smooth muscle cells by sodium butyrate, which inhibits PDGF-R kinase activity.

U.S. Pat. No. 5,476,851 (Myers, et al.) discloses Pyrazolo[3,4-g]quinoxaline compounds, as PDGF receptor protein tyrosine kinase inhibitors.

Compound SU-6668, an ATP competitive inhibitor, has been shown to inhibit PDGF-R autophosphorylation (Laird, et al., Cancer Res. 60:4152-4160, 2000].

WO01/79198 (Reich et al.) discloses amino-pyrazole compounds of the following formula that modulate and/or inhibit the activity of protein kinases.

WO0212242 (Fancelli et al.) discloses bicyclo-pyrazole compounds that are useful for treating diseases linked to disregulated protein kinases.

References to a number of substituted tricyclic pyrazole derivatives also include those disclosing use as: inhibitors of tyrosine kinase activity (WO 99/17769, WO 99/17770); cyclin dependent kinases inhibitors (WO 99/54308); selective estrogen receptor modulators (WO 00/07996); analgesics (U.S. Pat. No. 4,420,476); prophylaxis and therapy of diseases caused by rhinoviruses (U.S. Pat. No. 4,220,776; U.S. Pat. No. 4,140,785); analgesics/anti-inflammatory activity (U.S. Pat. No. 3,928,378; Schenone, Silvia et al. Farmaco (2000), 55(5), 383-388); cyan couplers for photographic dye (EP 0620489, JP 8022109); quinolines and naphthyridines as drugs (JP 6092963); and immunomodulators (JP 6100561); and hypoglycemic agents (Reddy, R. Raja et al., Indian Journal of Heterocyclic Chemistry (1998), 7(3), 189-192).

To date, STI-571 (GLEEVEC) is the only compound with significant PDGFR activity to reach market, although it is not a selective inhibitor of this enzyme. PDGF-R remains an extremely attractive target for the design of potent and selective small molecule inhibitors useful as therapeutic agents for the treatment of tumors and other cell proliferative disorders. Therefore, an equally attractive need exists for research tools useful for the development of PDGF-RTK inhibitors, for the identification of bimolecular targets in cells of therapeutic significance, profiling the selectivity of compounds, prediction of possible related toxicities and exploration of mechanisms of action in biological systems for therapeutic indications related to compounds of clinical interest, including PDGF-RTK inhibitor compounds.

### SUMMARY OF THE INVENTION

The present invention provides immobilized N-substituted tricyclic 3-aminopyrazole compounds of Formula 1 as tools for the identification of bimolecular targets in cells of therapeutic significance, profiling the selectivity of compounds, prediction of possible related toxicities and exploration of mechanisms of action in biological systems for therapeutic indications related to compounds. These agents can be used to identify biomolecules with the potential to interact with the immobilized reagent. The identified biomolecule may be then be used as a therapeutic target, serve as a marker of drug action, or alternatively desctibe an untoward or toxic potential of the immobilized agent.

### BRIEF DESCRIPTION OF THE FIGURES

FIG. 1 depicts a method of the present invention for the identification of a protein that covalently or non-covalently interacts with a compound of formula 1 containing a biotin substituent.

FIG. 2 depicts a method of the present invention for the identification of a protein that forms a reversible, non-covalent complex with a compound of formula 1.

FIG. 3 depicts a method of the present invention for the identification of a protein that forms a reversible, non-covalent complex with a compound of formula 1 containing a biotin substituent.

FIG. 4 depicts a method of the present invention for the identification of a protein that forms a reversible, non-covalent complex with a compound of formula 1 bound to a chip system.

FIGS. 5A and 5B depict a method of the present invention for identification of a protein that forms a reversible, non-covalent complex with a compound of formula 1 containing a biotin substituent using a protein chip system. FIG. 5A: the CATCH step is performed before the TRAP step. FIG. 5A: the TRAP step is performed before the CATCH step.

FIG. 6 shows a unique peak from a pH 9.0 fraction when a PS10 Chip-bound compound, Compound 6 of the present invention, was used to capture the associated proteins.

FIG. 7 shows a comparison of results obtained from using an ATP affinity column (top graph) and a PS10 Chip-bound compound (bottom graph).

FIG. 8 illustrates that Compound 124 of the invention forms a reversible, non-covalent complex with polymerizing tubulin: solid filed bars (compound 124); striped filed bars (control compound).

### DETAILED DESCRIPTION OF THE INVENTION

The present invention comprises a set of research tools and methods, whereby the N-substituted tricyclic 3-aminopyrazole compounds disclosed in U.S. patent application Ser. No. 10/438,152 and PCT/US/03/15193 are immobilized, and used for the characterization of PDGF-RTK inhibitors, for the identification of biomolecular targets in cells of therapeutic significance, profiling the selectivity of compounds, prediction of possible related toxicities and exploration of mechanisms of action in biological systems for therapeutic indications related to compounds of clinical interest, including PDGF-RTK inhibitor compounds or compounds with general anti-proliferative or other biological action.

1. Compounds of the Present Invention

1.A Definitions and Nomenclature

Unless otherwise noted, the term “alkyl” as used herein, whether used alone or as part of a substituent group, includes straight and branched chains having 1 to 10 carbon atoms, or any number within this range. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl, 2-methylpentyl, and the like. Unless otherwise noted, lower alkyl shall include straight and branched chains having 1 to 4 carbon atoms, or any number within this range.

Unless otherwise noted, the terms “alkoxy” or “alkyloxy” are used synonymously herein, and as used herein, whether used alone or as part of a substituent group, denotes an oxygen ether radical of the above described straight or branched chain alkyl groups. For example, alkoxy radicals include methoxy, ethoxy, n-propoxy, sec-butoxy, t-butoxy, n-hexyloxy and the like. Specific placement of the oxygen atom in relation to the alkyl portion is specified in the following manner, “—Oalkyl” or “-alkylO—”, to describe —OCH₃ and —CH₂O— respectively (wherein alkyl is methyl for purposes of the example).

Unless otherwise stated, “aryl,” employed alone or in combination with other terms (e.g., aryloxy, arylthioxy, arylalkyl), shall mean an aromatic ring structure comprising carbon atoms, for example, phenyl, naphthyl, fluorenyl, and the like.

As used herein, unless otherwise noted, “aralkyl” shall mean any lower alkyl group substituted with an aryl group such as phenyl, naphthyl and the like, for example, benzyl, phenylethyl, phenylpropyl, naphthylmethyl, and the like.

Unless otherwise noted, the term “cycloalkyl” as used herein, whether used alone or as part of a substituent group, shall mean any stable 3-10 membered, saturated ring system, for example cyclopropyl, cyclobutyl, cyclopentyl, cyclohexyl, cycloheptyl and cyclooctyl.

Unless otherwise noted, the term “partially unsaturated carbocycle” as used herein, whether used alone or as part of a substituent group, shall mean any stable 5-10 membered, partially unsaturated ring system, wherein the carbocycle contains, at least one unsaturated bond, for example cyclopentenyl, cyclohexenyl, cycloheptenyl, and the like.

Unless otherwise noted, the term “heteroaryl group” as used herein, whether used alone or as part of a substituent group, shall denote any five to ten membered monocyclic or bicyclic aromatic ring structure which containing carbon atoms and at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, pyrazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

As used herein, the term “heterocycloalkyl” shall denote any five to ten membered monocyclic or bicyclic, saturated or partially unsaturated ring structure containing C atoms and at least one heteroatom selected from the group consisting of O, N and S, optionally containing one to four additional heteroatoms independently selected from the group consisting of O, N and S. The monocyclic or bicyclic heteroalkyl group may be attached at any heteroatom or carbon atom of the ring such that the result is a stable structure. Examples of suitable monocyclic or bicyclic heteroalkyl groups include, but are not limited to, pyrrolinyl, pyrrolidinyl, dioxolanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 1,3-methylenedioxyphenyl (equivalent to benzofused dioxolyl), 1,4-ethylenedioxyphenyl (equivalent to benzofused dioxanyl), 2,3-dihydrobenzofuryl, and the like.

As used herein, unless otherwise noted, the term “benzo-fused heteroaryl” shall mean a bicyclic ring structure wherein one of the rings is phenyl and the other is a five to six membered heteroaryl. The benzo-fused heteroaryls are a subset of heteroaryls. Suitable examples include, but are not limited to, indolyl, isoindolyl, benzofuryl, benzothienyl, indazolyl, benzthiazolyl, quinolinyl, isoquinolinyl, cinnolinyl, phthalazinyl, quinazolinyl, pteridinyl, and the like.

As used herein, unless otherwise noted, the term “benzo-fused heterocycloalkyl” shall mean a bicyclic ring structure wherein one of the rings is phenyl and the other is a five to six membered heterocycloalkyl. The benzo-fused heterocycloalkyls are a subset of the heterocycloalkyl groups. Suitable examples include, but are not limited to, 1,3-benzodioxolyl (also known as 1,3-methylenedioxyphenyl), indolinyl, 1,4-benzodioxolanyl (also known as 1,4-ethylenedioxyphenyl), benzodihydrofuranyl, benzotetrahydropyranyl, benzodihydrothiophene and the like.

As used herein, unless otherwise noted, the term “benzo-fused cycloalkyl” shall mean a bicyclic ring structure wherein one of the rings is phenyl and the other is a three to eight membered cycloalkyl. Suitable examples include, but are not limited to indanyl, 1,2,3,4-tetrahydronaphthyl, 6,7,8,9,-tetrahydro-5H-benzocycloheptenyl, 5,6,7,8,9,10-hexahydro-benzocyclooctenyl, and the like.

As used herein, the term “linking group” is intended to refer to a divalent radical derived by, for example, the removal of at least one hydrogen atom from each of two different atoms, or the removal of two hydrogen atoms from a single atom, such that the two monovalent radical centers, or the single divalent radical center, form bonds with different atoms.

As used herein, the term “alkyldiyl” shall include straight and branched chain of 1 to 10 carbon atoms, or any number within this range, divalent or monovalent hydrocarbon radicals derived by the removal of one hydrogen atom from each of two different carbon atoms, or by the removal of two hydrogen atoms from a single carbon atom. Examples include methyldiyl (also referred to herein as methylene), and ethyldiyls (also referred to herein as ethylene), such as ethan-1,1-diyl, and ethan-1,2-diyl.

As used herein, the term “Matrix” refers to a support that is an insoluble, functionalized, polymeric material to which precursor compounds may be attached (via a linker) allowing them to be readily separated (by filtration, centrifugation, etc.) from excess reagents, soluble reaction by-products, or solvents. Matrix is also depicted herein with the following symbol:

In general, IUPAC nomenclature rules are used throughout this disclosure. Nomenclature for radical substituents is derived by first indicating the functionality having the point of attachment with a hyphen, followed by the adjacent functionality toward the terminal portion of the side chain, as in:
—(C₁₋₁₀)alkyl-C(O)NH—(C₁₋₁₀)alkyl-Phenyl
   or, when a lead hyphen is not used, by describing the terminal portion of the side chain first, followed by the adjacent functionality toward the point of attachment, as in:
phenyl-(C₁₋₁₀)alkylamido(C₁₋₁₀)alkyl, or phenylalkylamidoalkyl
   all three of, which refer to a radical of the formula:

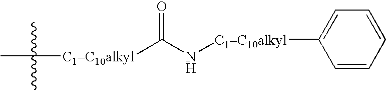

Where there are two points of attachment, for example in a linking group or a ring member, the two points of attachment are indicated with a lead hyphen and a final hypen. For example, the points of attachment of a linking group having two monvalent radical centers would be indicated as —(CH₂)₂— or —O(CH₂)₂— and the like; and the points of attachment of a linking group having a single divalent radical center would be indicated as —NH— or —N(C═O alkyl)- and the like. Points of attachment for an aromatic ring member would be indicated as —N—., —S— or —CH— and the like, for example.

Where the phrase “terminating with” is used, the point of attachment for the terminal substituent is indicated by the second dash. For example, for the phrase “—8C(═O)—(CH₂CH₂O—)₁₋₁₀ terminating with —H, methyl, ethyl, or benzyl” the point of attachment for the selected terminal substituent is the terminal oxygen, for example, —C(═O)—(CH₂CH₂OH) or —C(═O)—(CH₂CH₂OCH₂CH₂OCH₂CH₂OCH₃).

When a particular group is “substituted” (e.g., phenyl, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Compounds exemplified in the present invention were named according to nomenclature well known in the art, either using Autonom Version 2.2 (brand of nomenclature software provided in the ChemDraw Ultra® 7.0.1 Office Suite marketed by CambridgeSoft.com).

1.B Formula I

The compounds of the present invention comprise compounds of Formula I: 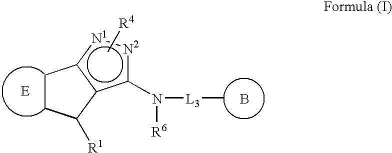
or an optical isomer, enantiomer, diastereomer, racemate, or pharmaceutically acceptable salt thereof, wherein: 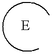
is selected from the group consisting of Formulae A-1, A-2 and A-3: 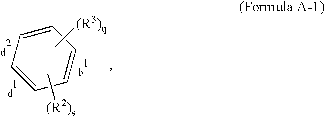
wherein Formula A-1 is attached on the b¹ side of Formula A-1 to the R¹-substituted ring of formula (I) and optionally substituted with one substituent selected from the group consisting of Formulae A-1-a, A-1-b and A-1-c: 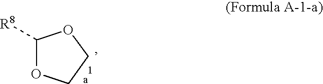
wherein Formula A-1-a is attached on the a¹ side to adjacent carbons on the d¹ or d² side of Formula A-1; 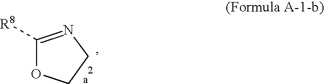
wherein Formula A-1-b is attached on the a² side to adjacent carbons on the d¹ or d² side of Formula A-1; and 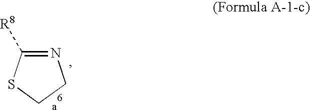
wherein Formula A-1-c is attached on the a⁶ side to adjacent carbons on the d¹ or d² side of Formula A-1;
wherein R⁸ is selected from the group consisting of hydrogen, alkyl, or L₄; 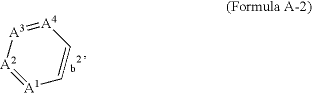
wherein Formula A-2 is attached on the b² side of Formula A-2 to the R¹-substituted ring of formula (I), and A¹, A², A³, A⁴ are (i) —N—; or (ii) —C—substituted with H or alkoxy, wherein the alkoxy may be optionally further substituted with alkoxy on a terminal carbon or up to 3 halogen atoms on a terminal carbon; provided that at least one and no more than two of A¹, A², A³, A⁴ are —N—; and 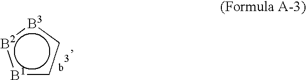
wherein Formula A-3 is attached on the b³ side of Formula A-3 to the R¹-substituted ring of formula (I), and B¹, B² and B³ are independently (i) —CH— optionally substituted with alkyl, aryl, alkoxy, or halogen, (ii) —S—; (iii) —O—; or (iv) —N—; provided that no more than one of B¹, B² or B³ is —S— or —O—, and, provided that when one of B¹, B² or B³ is —S— or —O—, then the adjacent ring members are not —S— or —O—;
   s is an integer from 0 to 2;
   q is an integer from 0 to 4; provided that when 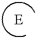
   is not substituted with Formulae A-1-a, A-1-b or A-1-c, the sum of q and s is an integer from 0 to 4, and when 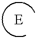
   is substituted with one of Formulae A-1-a, A-1-b, or A-1-c, the sum of q and s is an integer from 0 to 2;
   R¹ is selected from hydrogen, lower alkyl, —OH, alkoxy, -oxo-NH₂, —NH(alkyl), and —N(alkyl)₂;
   R² is selected from the group consisting of hydrogen, alkoxy, alkyl, alkenyl, alkylamino, amino, cyano, dialkylamino, halogen, halogenated alkyl, halogenated alkyloxy, hydroxylated alkyloxy, halogenated —SO₂alkyl, halogenated thioalkyl, hydroxy, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio, thioalkyl, —CO₂H, —C(═O)Oalkyl, —C(═O)NH₂, —C(═O)NH(alkyl), —C(═O)N(alkyl)₂, and L₄;
   R³ is independently selected from the group consisting of 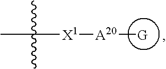
   X¹-A²⁰-Y¹-A²¹, —CO₂H, —C(═O)Oalkyl, —C(═O)NH₂, —C(═O)NH(alkyl), —C(═O)N(alkyl)₂, and L₄;
wherein X¹ and Y¹ are each independently absent or selected from the group consisting of: -(alkyl)C(═O)N(alkyl)-, —C(═O)N(alkyl)-, —C(═O)NH—, —C(═O)O—, —N(alkyl)-, —N(alkyl)C(═O)—, —N(alkyl)C(═O)NH—, —N(alkyl)C(═O)O—, —N(alkyl)SO₂—, —NH—, —NHC(═O)—, —NHC(═O)N(alkyl)-, —NHC(═O)NH, —NHC(═O)O—, —NHSO₂—, —O—, —OC(═O), —OC(═O)N(alkyl)-, —OC(═O)NH—, —OC(═O)O—, —S—, —SO—, —SO₂—, —SO₂N(alkyl)-, and —SO₂NH—;
   A²⁰, when present, is selected from alkyl or alkenyl; and
   A²¹ is selected from alkyl, alkenyl, or H;
wherein when A²⁰ or A²¹ is alkyl or alkenyl, the alkyl or alkenyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 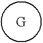
   is selected from the group consisting of aryl, cycloalkyl, partially unsaturated carbocycle, heteroaryl, heterocycloalkyl, nine to ten membered benzo-fused cycloalkyl, and nine to ten membered benzo-fused heterocycloalkyl; wherein, the aryl, cycloalkyl, partially unsaturated carbocycle, heteroaryl, heterocycloalkyl, benzo-fused cycloalkyl, or benzo-fused heterocycloalkyl, are optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂;
L₄ is selected from -M-K-J¹-Matrix or -M-K-J³-X-Matrix;
   wherein:
   M is selected from —C(═O)NH—, —(C═O)O—, —NHC(═O)—, —NH(C═O)NH—, —NH(C═O)O—, —O(C═O)NH—, —OC(═O)O—, —O(C═O)—, —O—, —NH—, —(CH₂)₁₋₃O—, —SS—, or —S—;
   K is selected from the group consisting of —(CH₂)ₘ(CH₂)ₙ(CH₂)ₚ—,
—(CH₂)ₘ(OCH₂CH₂)ₙ(CH₂)ₚ—,
(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)NH(CH₂)ᵣ₂]ₙ(CH₂)ₚ—,
(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘN H(C═O)CH₂CH₂(OCH₂CH₂)ₙNH(C═O)(CH₂)ₚ—,
—(CH₂)ₘ(C═O)NHCH₂CH₂(OCH₂CH₂)ₙ(C═O)NHCH₂(CH₂)ₚ—,
—(CH₂)ₘ(C═O)NHCH₂(CH₂)ₙ(C═O)NHCH₂(CH₂)ₚ—,
—(CH₂)ₘNH(CH₂)ₜ—, —(CH₂)ₘO(CH₂)ₜ—, —(CH₂)ₘS(CH₂)ₜ—,
—(CH₂)ₘS(═O)(CH₂)ₜ—, —(CH₂)ₘSO₂(CH₂)ₜ—,
—(CH₂)ₘNH(C═O)(CH₂)ₜ—,
—(CH₂)ₘ(C═O)NH(CH₂)ₜ—,
—(CH₂)ₘNH(C═O)NH(CH₂)ₜ—,
—(CH₂)ₘSS(CH₂)ₜ—,
—(CH₂)ₘ(SCH₂CH₂)ₙ(CH₂)ₚ—,
—(CH₂)ₘNH(C═O)CH₂CH₂(SCH₂CH₂)ₙNH(C═O)(CH₂)ₚ—,
—(CH₂)ₘ(C═O)NHCH₂CH₂(SCH₂CH₂)ₙ(C═O)NHCH₂(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—, and
—(CH₂)ₘ(OCH₂CH₂)ₙSS(CH₂)ₚ—;
   m is 1 to 7;
   n is 1 to 10;
   p is 0 to 7;
   r and r2 are 1 to 5;
   t is 1 to 7;
   J¹ is selected from —C(═O)NH—, —(C═O)O—, —NHC(═O)—, —NH(C═O)NH—, —NH(C═O)O—, —NH—, —O(C═O)NH—, —OC(═O)O—, —O(C═O)—, —O—, —(CH₂)₁₋₃O—, —SS—, —S—, —SS—, —SCH₂(CHOH)—, —CH═NNH—, 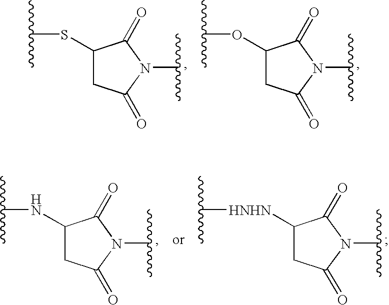
   J³ is selected from the group consisting of —NH—, —O—, —NHNH—, —NHNH═CH—, —NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —CH═NNH—, 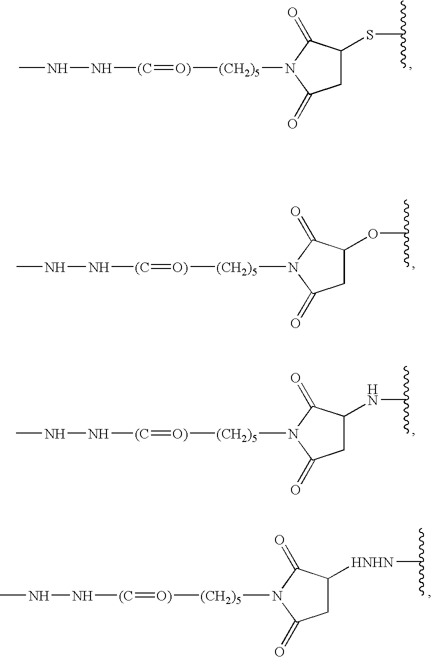
—NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHNH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHN H—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C═O)NHNH(C═O)NH—, and —NH(CH₂)₆—NH(C═O)CH₂CH₂SS—;
   provided that in L₄ the attachment of M to K to J¹ or J³ is not through a N—O or O—N bond, a N—S or S—N bond, an O—S or S—O bond, or an O—O bond;
   X is selected from biotin/avidin, biotin/streptavidin, iminobiotin/avidin, or iminobiotin/streptavidin, biotin/NeutrAvidin™, or iminobiotin/NeutrAvidin™;
   R⁴ is a substituent independently selected from:
(a) H; 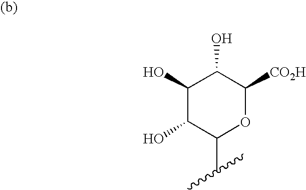
   provided that R⁶ is not 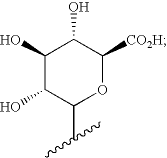
(c) —CH₂— or C₁₋₆alkenyl substituted with one group selected from: —H, -methyl, —Oalkyl, —CH₂OH, —CH(CH₃)OH, cyano, halogen, amino, —(CH₂)₁₄alkylamino, —(CH₂)₁₋₄dialkylamino, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(d) —C(═O)(CH₂CH₂O—)₁₋₁₀ terminating with H, methyl, ethyl, or benzyl;
(e) —C(═O)CH₂O(CH₂CH₂O—)₁₋₁₀ terminating with H, methyl, ethyl, or benzyl;
(f) —C(═O)alkyl, or —C(═O)(C₃₋₆)cycloalkyl, wherein said —C(═O)alkyl, and —C(═O)(C₃₋₆)cycloalkyl may be optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)(CH₂)₁₋₃aryl, —C(═O)aryl, —C(═O)(CH₂)₁₋₃heteroaryl, or —C(═O)heteroaryl, wherein said —C(═O)(CH₂)₁₋₃aryl, —C(═O)aryl, —C(═O)(CH₂)₁₋₃heteroaryl, and —C(═O)heteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh) —C(═O)alkylOC(═O)alkyl- terminating with —OH, —Oalkyl, —NH₂, —NHalkyl, —N(alkyl)₂, or heterocycloalkyl;
(i) —C(═O)O(CH₂CH₂O—)₁₋₁₀ terminating with H, methyl, ethyl, or benzyl;
(j) —C(═O)Oalkyl, or —C(═O)O(C₃₋₆)cycloalkyl, wherein said —C(═O)Oalkyl, and —C(═O)O(C₃₋₆)cycloalkyl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)O(CH₂)₁₋₃aryl, —C(═O)Oaryl, —C(═O)O(CH₂)₁₋₃heteroaryl, or —C(═O)Oheteroaryl, wherein said —C(═O)O(CH₂)₁₋₃aryl, —C(═O)Oaryl, —C(═O)O(CH₂)₁₋₃heteroaryl, or —C(═O)Oheteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH₂, —C(═O)NH(C₁₋₂₀)alkyl, —C(═O)NH(C₃₋₆)cycloalkyl, or —C(═O)N(alkyl)₂, wherein said —C(═O)NH(C₁₋₂₀)alkyl, —C(═O)NH(C₃₋₆)cycloalkyl, and —C(═O)N(alkyl)₂ may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NHalkyl, —N(alkyl)₂, halogen or nitrile;
(n) —C(═O)NH(CH₂)₁₋₃aryl, —C(═O)NHaryl, —C(═O)NH(CH₂)₁₋₃heteroaryl, or —C(═O)NHheteroaryl, wherein said —C(═O)NH(CH₂)₁₋₃aryl, —C(═O)NHaryl, —C(═O)NH(CH₂)₁₋₃heteroaryl, and —C(═O)NHheteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(q) —C(═S)NHalkyl;
(r) —C(═S)N(alkyl)₂;
(s) —SO₂NH₂;
(t) —SO₂NHalkyl;
(u) —SO₂N(alkyl)₂;
(v) —P(═O)(OCH₃)₂;
(w) —P(═O)(OCH₂CH₃)₂; or
(x) L₅
   L₅ is selected from -M¹-K-J¹-Matrix or -M¹-K-J³-X-Matrix
   wherein:
   M¹ is selected from —C(═O)—, —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄O(C═O)NH—, —(CH₂)₂₋₄OC(═O)—, —(CH₂)₂₋₄(C═O)O—, —(CH₂)₂₋₄S—, —(CH₂)₂₋₄SS—, or —(CH₂)₂₋₄O—;
      provided that in L₅ the attachment of M¹ to K to J¹ or J³ is not through a N—O or O—N bond, a N—S or S—N bond, an O—S or S—O bond, or an O—O bond;
   R⁶ is selected from the group consisting of H and 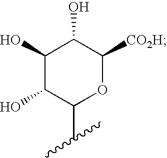
   provided that if R⁴ is 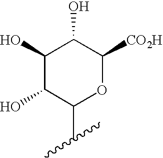
   then R⁶ is H;
   L₃ is absent or is a linking group selected from the group consisting of alkylidyl, carbonyl, -alkylidyl-C(═O)—, —C(═O)-alkylidyl-, -alkylidylC(═O)alkylidyl-, or —SO₂—;
   B is selected from the group consisting of an aryl, a heteroaryl, a nine to ten membered benzo-fused cycloalkyl, a nine to ten membered benzo-fused heterocycloalkyl, —CH(R⁹)aryl, and —CH(R⁹)heteroaryl; wherein the aromatic portion of said B is optionally substituted with R⁵; and wherein R⁹ is a substituent selected from hydrogen, alkyl, or cycloalkyl, wherein said alkyl is optionally substituted with alkylamino, amino, cyano, dialkylamino, halogenated alkyl, halogenated alkyloxy, —SO₂alkyl, or hydroxy;
   R⁵ is independently selected from the group consisting of L₄, alkyl, alkylamino, alkyloxy, amino, —C(═O)NH₂, —C(═O)NH(alkyl), —C(═O)N(dialkyl), —C(═O)Oalkyl, —C(═O)OH, —CH₂OH, cyano, dialkylamino, halogen, halogenated alkyl, halogenated alkyloxy, halogenated SO₂-alkyl, halogenated thioalkyl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, —SO₂NH₂, thio, thioalkyl, 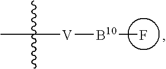
   and -V-B¹⁰-W-B²⁰; wherein,
V and W are each independently absent or selected from the group consisting of: —C(═O), —C(═O)N(alkyl)-, —C(═O)NH—, —C(═O)O—, —N(alkyl)-, —N(alkyl)C(═O)—, —N(alkyl)C(═O)N(alkyl)-, —N(alkyl)C(═O)NH—, —N(alkyl)C(═O)O—, —N(alkyl)SO₂—, —NH—, —NHC(═O)—, —NHC(═O)N(alkyl)-, —NHC(═O)NH—, —NHC(═O)O—, —NHSO₂—, —O—, —OC(═O), —OC(═O)N(alkyl)-, —OC(═O)NH—, —OC(═O)O—, —S—, —SO—, —SO₂—, —SO₂N(alkyl)- and —SO₂NH—;
B¹⁰ is absent or selected from alkyl or alkenyl;
B²⁰ is absent or selected from alkyl, alkenyl, or H;
wherein, when B¹⁰ or B²⁰ is alkyl or alkenyl, the alkyl or alkenyl group may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; and 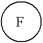
   is selected from the group consisting of: an aryl, a cycloalkyl, a partially unsaturated carbocycle, a heteroaryl, a heterocycloalkyl, a nine to ten membered benzo-fused cycloalkyl, and a nine to ten membered benzo-fused heterocycloalkyl,
wherein, the aryl, cycloalkyl, partially unsaturated carbocycle, heteroaryl, heterocycloalkyl, benzo-fused cycloalkyl, or benzo-fused heterocycloalkyl, is optionally substituted with one or more substituents independently selected from: alkoxy, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl;
   provided that at least one, and no more than one, L₄ or L₅ is present.

### 1.C EMBODIMENTS

Further embodiments of the present invention include compounds of Formula (I) wherein E, R⁸, R¹, R², R³, R⁴, L₄, L₅, R⁵, R⁶, L³, and B vary as set forth below individually and combinations of the variations thereof.

An embodiment of the present invention includes compounds of Formula (I) wherein: 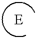
is selected from the group consisting of Formulae A-1, A-2 and A-3: 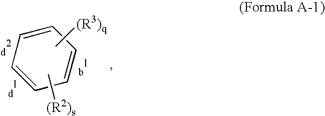
wherein Formula A-1 is attached on the b¹ side of Formula A-1 to the R¹ substituted ring of formula (I) and optionally substituted with one substituent selected from the group consisting of Formulae A-1-a, A-1-b and A-1-c: 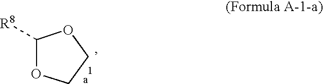
wherein Formula A-1-a is attached on the a¹ side to adjacent carbons on the d¹ or d² side of Formula A-1; 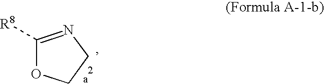
wherein Formula A-1-b is attached on the a² side to adjacent carbons on the d¹ or d² side of Formula A-1; and 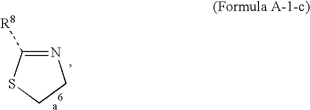
wherein Formula A-1-c is attached on the a⁶ side to adjacent carbons on the d¹ or d² side of Formula A-1;
wherein R⁸ is hydrogen, lower alkyl, or L₄; 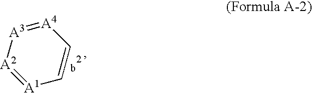
wherein Formula A-2 is attached on the b² side of Formula A-2 to the R¹ substituted ring of formulae (I), and one or two of A¹, A², A³, A⁴ are —N—; the remainder being —C— substituted with H or alkoxy, wherein the alkoxy may be optionally further substituted with alkoxy on a terminal carbon or up to 3 halogen atoms on a terminal carbon; and 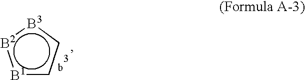
wherein Formula A-3 is attached on the b³ side of Formula A-3 to the R¹ substituted ring of formulae (I), and B¹, B² and B³ are independently (i) —CH— optionally substituted with C₁₋₄alkyl, aryl, alkoxy, or halogen, (ii)-S—, (iii)-O— or (iv)-N—; provided that no more than one of B¹, B² or B³ is —S— or —O—, and, provided that when one of B¹, B² or B³ is —S— or —O—, then the adjacent ring members are not —S— or —O—;

An embodiment of the present invention includes compounds of Formula (I) wherein: 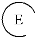
is selected from the group consisting of Formulae A-1, A-2 and A-3: 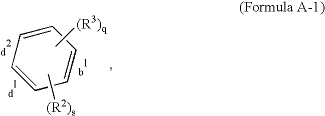
wherein Formula A-1 is attached on the b¹ side of Formula A-1 to the R¹ substituted ring of formulae (I) and optionally substituted with one substituent selected from the group consisting of Formulae A-1-a, A-1-b and A-1-c: 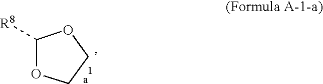
wherein Formula A-1-a is attached on the a¹ side to adjacent carbons on the d¹ or d² side of Formula A-1; 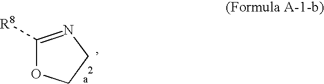
wherein Formula A-1-b is attached on the a² side to adjacent carbons on the d¹ or d² side of Formula A-1; and 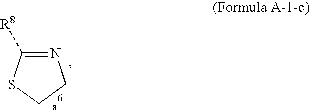
wherein Formula A-1-c is attached on the a⁶ side to adjacent carbons on the d¹ or d² side of Formula A-1;
wherein R⁸ is hydrogen, lower alkyl, or L₄; 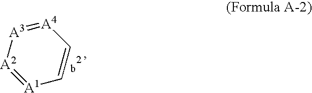
wherein Formula A-2 is selected from the group consisting of pyridyl and pyrimidinyl; is attached on the b² side of Formula A-2 to the R¹ substituted ring of formulae (I); and is optionally substituted on a carbon ring member with H or alkoxy, wherein the alkoxy may be optionally further substituted with alkoxy on a terminal carbon or up to 3 halogen atoms on a terminal carbon; and 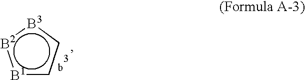
wherein Formula A-3 is selected from the group consisting of thienyl, isoxazolyl and furyl; is attached on the b³ side of Formula A-3 to the R¹ substituted ring of formulae (I), and is optionally substituted on a carbon ring member with C₁₋₄alkyl, aryl, alkoxy, or halogen.

An embodiment of the present invention includes compounds of Formula (I) wherein 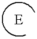
is selected from the group consisting of: 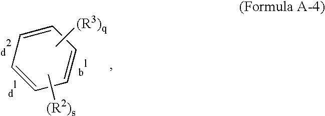
wherein Formula A-4 is attached on the b¹ side of Formula A-4 to the R¹ substituted ring of formulae (I); 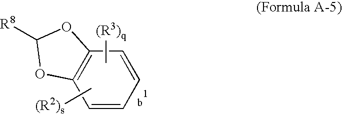
wherein Formula A-5 is attached on the b¹ side of Formula A-5 to the R¹ substituted ring of Formula (I); wherein R⁸ is hydrogen, lower alkyl, or L₄; 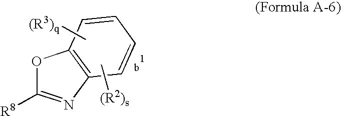
wherein Formula A-6 is attached on the b¹ side of Formula A-6 to the R¹ substituted ring of Formula (I); and 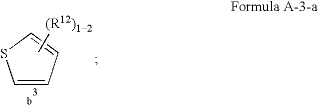
wherein Formulae A-3-a is attached on the b³ side of Formulae A-3-a to the R¹ substituted ring of formula (I), wherein R¹² is independently selected from H, methyl, phenyl, ethoxy, chloro or fluoro; and wherein
q is an integer from 0 to 4; provided that when 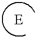
   is Formula A-4, the sum of q and s is an integer from 0 to 4, and when 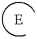
   is Formulae A-5 or A-6, the sum of q and s is an integer from 0 to 2.

An embodiment of the present invention includes compounds of Formula (I) wherein 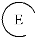
is selected from the group consisting of: 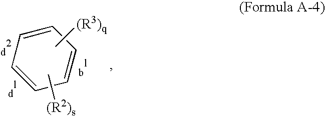
wherein Formula A-4 is attached on the b¹ side of Formula A-4 to the R¹ substituted ring of formulae (I); 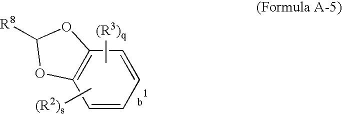
wherein Formula A-5 is attached on the b¹ side of Formula A-5 to the R¹ substituted ring of Formula (I); wherein R⁸ is hydrogen, lower alkyl, or L₄; and 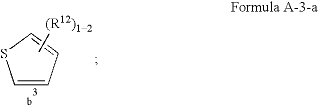
wherein Formulae A-3-a is attached on the b³ side of Formulae A-3-a to the R¹ substituted ring of formula (1), wherein R¹² is independently selected from H, methyl, phenyl, ethoxy, chloro or fluoro; and wherein
q is an integer from 0 to 4; provided that when 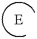
   is Formula A-4, the sum of q and s is an integer from 0 to 4, and when 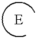
   is Formula A-5, the sum of q and s is an integer from 0 to 2.

An embodiment of the present invention includes compounds of Formula (I) wherein 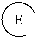
is: 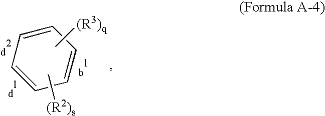
wherein Formula A-4 is attached on the b¹ side of Formula A-4 to the R¹ substituted ring of formulae (I); and wherein
q is an integer from 0 to 4; provided that when 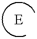
   is Formula A-4, the sum of q and s is an integer from 0 to 4.

An embodiment of the present invention includes compounds of Formula (I) wherein
R¹ is selected from hydrogen, lower alkyl, —OH, alkoxy, —NH₂, —NH(alkyl), and —N(alkyl)₂.

An embodiment of the present invention includes compounds of Formula (I) wherein
R¹ is selected from hydrogen, lower alkyl, —OH, or alkoxy.

An embodiment of the present invention includes compounds of Formula (I) wherein
R¹ is selected from hydrogen or lower alkyl.

An embodiment of the present invention includes compounds of Formula (I) wherein
R¹ is hydrogen.

An embodiment of the present invention includes compounds of Formula (I) wherein:
R² is selected from the group consisting of hydrogen, alkoxy, alkyl, alkylamino, amino, cyano, dialkylamino, halogen, halogenated alkyl, hydroxylated alkyloxy, halogenated alkyloxy, halogenated —SO₂alkyl, hydroxy, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)N(alkyl)₂, —OC(═O)NHalkyl, —SO₂alkyl, thio, thioalkyl, —CO₂H, —C(═O)Oalkyl, —C(═O)NH₂, —C(═O)NH(alkyl), —C(═O)N(alkyl)₂, and L₄.

An embodiment of the present invention includes compounds of Formula (I) wherein:
R² is independently selected from the group consisting of: alkoxy, alkyl, alkylamino, amino, cyano, dialkylamino, halogen, halogenated alkyl, hydroxylated alkyloxy, halogenated alkyloxy, hydroxy, —NHC(═O)alkyl, —NHC(═O)NHalkyl, —NHC(═O)Oalkyl, —NHSO₂alkyl, thioalkyl, and L₄.

A embodiment of the present invention includes compounds of Formula (I) wherein:
R² is independently selected from the group consisting of: alkoxy, alkyl, amino, cyano, dialkylamino, halogen, halogenated alkyl, halogenated alkyloxy, hydroxylated alkyloxy, hydroxy, —NHC(═O)alkyl, and L₄.

A embodiment of the present invention includes compounds of Formula (I) wherein:
R² is independently selected from the group consisting of: methoxy, ethoxy, isopropoxy, methyl, amino, cyano, N,N-dimethyl-amino, bromo, chloro, fluoro, trifluoromethyl, trifluoromethoxy, (3-hydroxy)-prop-1-oxy, hydroxy, N-(1-oxo-ethyl)-amino, and L₄.

A embodiment of the present invention includes compounds of Formula (I) wherein:
R³ is independently selected from the group consisting of 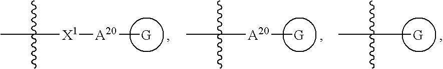
   -X¹-A²⁰-Y¹-A²¹, -X¹-A²⁰-A²¹ and -X¹-A²¹, —CO₂H, —C(═O)Oalkyl, —C(═O)NH₂, —C(═O)NH(alkyl), —C(═O)N(alkyl)₂, and L₄;

A embodiment of the present invention includes compounds of Formula (I) wherein:

wherein X¹ and Y¹ are each independently absent or selected from the group consisting of: —C(═O)NH—, —C(═O)O—, —NH—, —NHC(═O)—, —NHC(═O)NH, —NHC(═O)O—, —NHSO₂—, —O—, —OC(═O), —OC(═O)NH—, —OC(═O)O—, —S—, —SO—, —SO₂— and —SO₂NH—.

An embodiment of the present invention includes compounds of Formula (I):

wherein X¹ and Y¹ are each independently absent or selected from the group consisting of: —NH—, —O—, —SO₂— and —SO₂NH—.

An embodiment of the present invention includes compounds of Formula (I), wherein X¹ and Y¹ are each independently absent or —O—.

An embodiment of the present invention includes compounds of Formula (I), wherein X¹ is absent or —O—.

An embodiment of the present invention includes compounds of Formula (I), wherein Y¹ is absent.

An embodiment of the present invention includes compounds of Formula (I), wherein:
A²⁰ is absent or alkyl; and
   wherein when A²⁰ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl.

An embodiment of the present invention includes compounds of Formula (I) wherein:
A²⁰ is absent or alkyl; and
   wherein when A²⁰ is alkyl, the alkyl may be optionally substituted with one or more groups independently selected from: alkoxy, alkylamino, amino, dialkylamino, halogen, halogenated alkoxy, hydroxy, —NHC(═O)NH₂, —NHSO₂alkyl, —SO₂alkyl or thioalkyl.

An embodiment of the present invention includes compounds of Formula (I) wherein:
A²⁰ is absent or selected from methyl, ethyl, propyl or isopropyl; wherein methyl, ethyl, propyl or isopropyl are optionally substituted with one or more groups independently selected from: alkoxy, dialkylamino or hydroxy.

An embodiment of the present invention includes compounds of Formula (I) wherein:
A²⁰ is absent or selected from methyl, ethyl, propyl or isopropyl; wherein methyl, ethyl, propyl or isopropyl are optionally substituted with one or more groups independently selected from methoxy, dimethyl-amino or hydroxy.

An embodiment of the present invention includes compounds of Formula (I) wherein:
A²¹ is selected from alkyl, alkenyl, or H; and
   wherein when A²¹ is alkyl or alkenyl, the alkyl or alkenyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)N Halkyl, —OC(═O)Oalkyl, —SO₂alkyl, thio or thioalkyl.

An embodiment of the present invention includes compounds of Formula (I) wherein:
A²′ is selected from alkyl, alkenyl, or H; and
   wherein when A²′ is alkyl or alkenyl, the alkyl or alkenyl may be optionally substituted with one or more groups independently selected from: alkoxy, alkylamino, amino, dialkylamino, halogen, halogenated alkoxy, hydroxy, —NHC(═O)NH₂, —NHSO₂alkyl or thioalkyl.

An embodiment of the present invention includes compounds of Formula (I) wherein A²¹ is H.

An embodiment of the present invention includes compounds of Formula (I) wherein 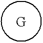
is selected from the group consisting of aryl, cycloalkyl, partially unsaturated carbocycle, heteroaryl and heterocycloalkyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂.

An embodiment of the present invention includes compounds of Formula (I) wherein 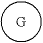
is selected from the group consisting of aryl, heteroaryl and heterocycloalkyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂.

An embodiment of the present invention includes compounds of Formula (I) wherein 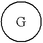
is selected from the group consisting of phenyl, imidazolyl, pyrrolidinyl, piperidinyl and morpholinyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂.

An embodiment of the present invention includes compounds of Formula (I) wherein 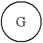
is selected from the group consisting of phenyl, imidazolyl, pyrrolidinyl, piperidinyl and morpholinyl optionally substituted with one or more substituents independently selected from halogen, hydroxy, amino, nitro, alkyl, halogenated alkyl, alkoxy, halogenated alkoxy, alkylamino, dialkylamino, —NHSO₂alkyl or —SO₂alkyl.

An embodiment of the present invention includes compounds of Formula (I) wherein 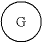
is selected from the group consisting of phenyl, imidazolyl, pyrrolidinyl, piperidinyl and morpholinyl optionally substituted with one or more substituents independently selected from chloro, fluoro, hydroxy or alkyl.

A preferred embodiment of the present invention includes compounds of Formula (I) wherein:
L₄ is selected from -M-K-J′-Matrix or -M-K-J³-X-Matrix;
   wherein M is selected from —C(═O)NH—, —O—, —NHC(═O)O—, —S—, or —SS—;
   and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)NH(CH₂)ᵣ₂]ₙ(CH₂)ₚ—,
—(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—,
—(CH₂)ₘ(OCH₂CH₂)ₙSS(CH₂)ₚ—; and
   wherein J¹ is selected from —C(═O)NH—, —(C═O)O—, —NHC(═O)—, —NH(C═O)NH—, —NH(C═O)O—, —O(C═O)NH—, —OC(═O)O—, —O(C═O)—, —O—, —NH—, —(CH₂)₁₋₃O—, —SS—, —S—, —SCH₂(CHOH)—, —CH═NNH—, 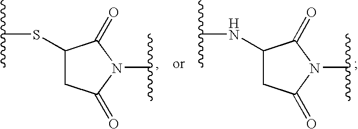
      and
   wherein J³ is selected from the group consisting of —NH—, —O—, —NHNH—,
—NHNH═CH—, —NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHNH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C—O)NHNH(C═O)NH—, and —NH(CH₂)₆—NH(C═O)CH₂CH₂SS—;
   provided that in L₄ the attachment of M to K to J¹ or J³ is not through a N—O or O—N bond, a N—S or S—N bond, an O—S or S—O bond, or an O—O bond.

A more preferred embodiment of the present invention includes compounds of Formula (I) wherein:
L₄ is selected from -M-K-J¹-Matrix or -M-K-J³-X-Matrix;
   wherein M is selected from —C(═O)NH— or —O—;
   and wherein K is, selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ(OCH₂CH₂)ₙNHC(═O)(CH₂)ₚ—,
—(CH₂)ₘ(OCH₂CH₂)ₙSS(CH₂)ₚ—; and
   wherein J¹ is selected from —NH—, —NHC(═O)O—, —OC(═O)—, —C(═O)O—, —C(═O)NH—, or —NHC(═O)—, —CH═NNH—, 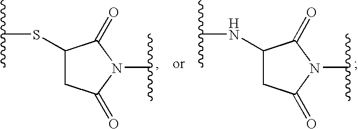
   and
   J³ is selected from —NH—, —NHC(═O)O—, —OC(═O)—, —C(═O)O—, —C(═O)NH—, or —NHC(═O)—;
      provided that in L₄ the attachment of M to K to J¹ or J³ is not through a N—O or O—N bond, a N—S or S—N bond, an O—S or S—O bond, or an O—O bond.

An embodiment of the present invention includes compounds of Formula (I) wherein:
R⁴ is independently selected from:
(a) H;
(b) 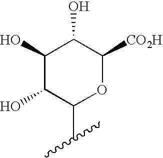
   provided that R⁶ is not 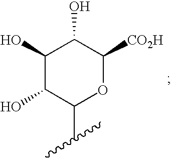
(c) —CH₂— substituted with one group selected from: —H, -methyl, —Oalkyl, —CH₂OH, —CH(CH₃)OH, —O(C═O)alkyl, —(C═O)OH, —C(═O)Oalkyl, —C(═O)Oaryl, —C(═O)Oheteroaryl, —(C═O)NH₂, —(C═O)N Halkyl, —(C═O)N(alkyl)₂, —C(═O)alkyl, -phenyl-OCH₃ or -phenyl-OC(═O)alkyl;
(d) —C(═O)(CH₂CH₂O—)₁₋₁₀ terminating with H;
(e) —C(═O)CH₂O(CH₂CH₂O—)₁₋₁₀ terminating with H;
(f) —C(═O)alkyl optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)(CH₂)₁₋₃aryl or —C(═O)aryl, wherein said —C(═O)aryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh)-C(═O)alkylOC(═O)alkyl- terminating with —OH, —Oalkyl, —NH₂, —NHalkyl or —N(alkyl)₂;
(i) —C(═O)O(CH₂CH₂O—)₁₋₁₀ terminating with H, methyl, ethyl, or benzyl;
(j) —C(═O)Oalkyloptionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)O(CH₂)₁₋₃aryl or —C(═O)Oaryl, wherein said —C(═O)Oaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH₂, —C(═O)NH(C₁₋₂₀)alkyl, or —C(═O)N(C₁₋₂₀alkyl)₂, wherein said —C(═O)NH(C₁₋₂₀)alkyl, may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NHalkyl, —N(alkyl)₂, halogen or nitrile;
(n) —C(═O)NH(CH₂)₁₋₃aryl or —C(═O)NHaryl, wherein said —C(═O)NHaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(q) —C(═S)NHalkyl;
(r) —C(—S)N(alkyl)₂;
(s) —SO₂NH₂;
(t) —SO₂NHalkyl;
(u) —SO₂N(alkyl)₂;
(v) —P(═O)(OCH₃)₂;
(w) —P(═O)(OCH₂CH₃)₂; or
(x) L₅.

An embodiment of the present invention includes compounds of Formula (I) wherein:
R⁴ is independently selected from:
(a) H;
(b) 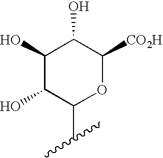
   provided that R⁶ is not 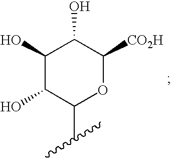
(c) —CH₂— substituted with one group selected from: —H, -methyl, —Oalkyl, —CH₂OH, —CH(CH₃)OH, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(f) —C(═O)alkyl optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)(CH₂)₁₋₃aryl or —C(═O)aryl, wherein said —C(═O)aryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh) —C(═O)alkylOC(═O)alkyl- terminating with —OH, —Oalkyl, —NH₂, —NHalkyl or —N(alkyl)₂;
(i) —C(—O)O(CH₂CH₂O—)₁₋₁₀ terminating with H, methyl, ethyl, or benzyl;
(j) —C(═O)Oalkyloptionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)O(CH₂)₁₋₃aryl or —C(═O)Oaryl, wherein said —C(═O)Oaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH₂, —C(═O)NH(C₁₋₂₀)alkyl, or —C(═O)N(C₁₋₂₀alkyl)₂, wherein said —C(═O)NH(C₁₋₂₀)alkyl, may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NHalkyl, —N(alkyl)₂, halogen or nitrile;
(n) —C(═O)NH(CH₂)₁₋₃aryl or —C(═O)NHaryl, wherein said —C(═O)NHaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(u) —SO₂N(alkyl)₂; and
(w) —P(═O)(OCH₂CH₃)₂; or
(x) L₅.

An embodiment of the present invention includes compounds of Formula (I) wherein
R⁴ is independently selected from:
(a) H;
(b) 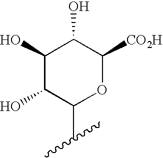
   provided that R⁶ is not 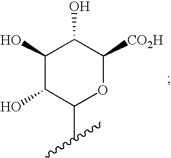
(c) —CH₂— substituted with one group selected from: —H, -methyl, —Oalkyl, —CH₂OH, —CH(CH₃)OH, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(f) —C(═O)alkyl optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)aryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh) —C(═O)alkylOC(═O)alkyl- terminating with —OH, —Oalkyl, —NH₂, —NHalkyl or —N(alkyl)₂;
(i) —C(—O)O(CH₂CH₂O—)₁₋₁₀ terminating with H, methyl, ethyl, or benzyl;
(j) —C(═O)Oalkyloptionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)Oaryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH(C₁₋₂₀)alkyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NHalkyl, —N(alkyl)₂, halogen or nitrile;
(n) —C(═O)NHaryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(u) —SO₂N(alkyl)₂
(w) —P(═O)(OCH₂CH₃)₂; or
(x) L₅

An embodiment of the present invention includes compounds of Formula (I) wherein
R⁴ is independently selected from:
(a) H;
(b) 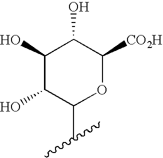
   provided that R⁶ is not 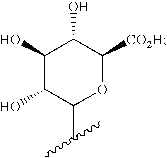
(c) —CH₂— substituted with one group selected from: —H, -methyl, —Oalkyl, —CH₂OH, —CH(CH₃)OH, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(f) —C(═O)alkyl optionally substituted with one or more groups independently selected from: —OH, —Oalkyl, —Oalkyl-phenyl, —NH₂, —NHalkyl, —N(alkyl)₂, heterocycloalkyl, —NHC(═O)alkyl, —NHSO₂alkyl, or —OC(═O)alkyl;
(g) —C(═O)aryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or pyrrolidinyl;
(hh) —C(═O)alkylOC(═O)alkyl- terminating with —OH, —Oalkyl, —NH₂, —NHalkyl or —N(alkyl)₂;
(i) —C(═O)O(CH₂CH₂O—)₁₋₁₀ terminating with H, methyl, ethyl, or benzyl;
(j) —C(═O)Oalkyloptionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)Oaryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH(C₁₋₂₀)alkyl optionally substituted with one or more groups independently selected from: —OH, —Oalkyl, —NH₂, —NHalkyl, —N(alkyl)₂, pyrrolidinyl, morpholinyl, —NHC(═O)alkyl, —NHSO₂alkyl, —OC(═O)alkyl, —OC(═O)alkenyl, —NHC(═O)phenyl, —C(═O)OH, —C(═O)Oalkyl, —C(═O)NH₂, —C(═O)NHalkyl, or —C(═O)N(alkyl)₂; and, wherein the phenyl portion of said —NHC(═O)phenyl may be optionally substituted with one or more groups independently selected from: alkyl, —OH, —Oalkyl, —NH₂, —NHalkyl, —N(alkyl)₂, halogen or nitrile;
(n) —C(═O)NHaryl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)phenyl; wherein the phenyl portion of said —C(═O)phenyl may be optionally substituted with one or more groups independently selected from: alkyl, —OH, —Oalkyl, —NH₂, —NHalkyl, —N(alkyl)₂, halogen or nitrile;
(p) —C(═S)NH₂;
(u) —SO₂N(alkyl)₂
(v) —P(═O)(OCH₂CH₃)₂; or
(x) L₅

An embodiment of the present invention includes compounds of Formula (I) wherein:
R⁴ is independently selected from:
(a) H;
(b) 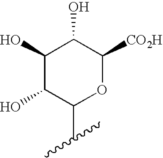
   provided that R⁶ is not 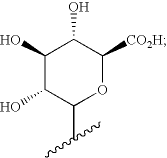
(c) —CH₂— substituted with one group selected from: —H, -methyl, —Oalkyl, —CH₂OH, —CH(CH₃)OH, —O(C═O)alkyl, —C(═O)Oalkyl, —(C═O)NH₂, —C(═O)alkyl or -phenyl-OC(═O)alkyl;
(f) —C(═O)alkyl optionally substituted with one or more groups independently selected from: —OH, —Oalkyl, —Oalkyl-phenyl or —OC(═O)alkyl;
(g) —C(═O)phenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(h) —C(═O)(CH₂)₁₋₆C(═O)— terminating with —OH, —Oalkyl, —NH₂, —NHalkyl, —N(alkyl)₂, or pyrrolidinyl;
(hh) —C(═O)alkylOC(═O)alkyl- terminating with —Oalkyl;
(i) —C(═O)O(CH₂CH₂O—)₁₋₁₀ terminating with H or benzyl;
(j) —C(═O)Oalkyloptionally substituted with one or more —Oalkyl groups;
(k) —C(═O)Ophenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(l) —C(—O)NH(CH₂CH₂O—)₁₋₁₀ terminating with —H;
(m) —C(═O)NH(C₁₋₂₀)alkyl optionally substituted with one or more groups independently selected from: —NH₂, —NHalkyl, —N(alkyl)₂, pyrrolidinyl, morpholinyl, —NHC(═O)alkyl, —OC(═O)alkenyl, —NHC(═O)phenyl or —C(═O)Oalkyl; and, wherein the phenyl portion of said —NHC(═O)phenyl may be optionally substituted with one or more groups independently selected from: alkyl, —OH, —Oalkyl, —NH₂, —NHalkyl, —N(alkyl)₂, halogen or nitrile;
(n) —C(═O)NHphenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, and —C(═O)phenyl; wherein the phenyl portion of said —C(—O)phenyl may be optionally substituted with one or more groups independently selected from: alkyl, —OH, —Oalkyl, —NH₂, —NHalkyl, —N(alkyl)₂, halogen and nitrile;
(p) —C(═S)NH₂;
(u) —SO₂N(alkyl)₂
(v) —P(═O)(OCH₂CH₃)₂; or
(x) L₅.

An embodiment of the present invention includes compounds of Formula (I) wherein
R⁴ is independently selected from:
(a) H;
(b) 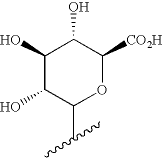
   provided that R⁶ is not 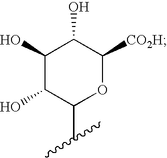
(c) —CH₂— substituted with one group selected from: —H, -methyl, —Oalkyl, —CH₂OH, —CH(CH₃)OH, —O(C═O)alkyl, —C(═O)Oalkyl, —(C═O)NH₂, —C(═O)alkyl or -phenyl-OC(═O)alkyl;
(f) —C(═O)alkyl optionally substituted with one or more groups independently selected from: —OH, —Oalkyl, —Oalkyl-phenyl or —OC(═O)alkyl;
(g) —C(═O)phenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, and —OC(═O)alkyl;
(h) —C(═O)(CH₂)₁₋₆C(═O)— terminating with —OH, —Oalkyl, —NH₂, —NHalkyl, —N(alkyl)₂, or pyrrolidinyl;
(hh) —C(═O)alkylOC(═O)alkyl- terminating with —Oalkyl;
(i) —C(═O)O(CH₂CH₂O—)₁₋₁₀ terminating with H or benzyl;
(j) —C(═O)Oalkyloptionally substituted with one or more —Oalkyl groups;
(k) —C(═O)Ophenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, and —OC(═O)alkyl;
(l) —C(═O)NH(CH₂CH₂O—)₁₋₁₀ terminating with —H;
(m) —C(═O)NH(C₁₋₂₀)alkyl optionally substituted with one or more groups independently selected from: —NH₂, —NHalkyl, —N(alkyl)₂, pyrrolidinyl, morpholinyl, —NHC(═O)alkyl, —OC(═O)alkenyl, —NHC(═O)phenyl or —C(═O)Oalkyl; and, wherein the phenyl portion of said —NHC(═O)phenyl may be optionally substituted with one or more groups independently selected from: alkyl, —OH, —Oalkyl, —NH₂, —NHalkyl, —N(alkyl)₂, halogen and nitrile;
(n) —C(═O)NHphenyl optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, chloro, fluoro, nitrile, and —OC(═O)alkyl;
(o)—C(—O)NHCH₂CH₂NH(CH₂CH₂NH—)₀₋₃ terminating with —CH₂CH₂OH and —C(═O)phenyl; wherein the phenyl portion of said —C(═O)phenyl may be optionally substituted with one or more groups independently selected from: alkyl, —OH, —Oalkyl, —NH₂, —NHalkyl, —N(alkyl)₂, halogen and nitrile;
(p) —C(═S)NH₂;
(u) —SO₂N(alkyl)₂
(w) —P(═O)(OCH₂CH₃)₂; or
(x) L₅.

An embodiment of the present invention includes compounds of Formula (I) wherein
R⁴ is independently selected from:
(a) H;
(b) 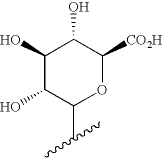
   provided that R⁴ is not 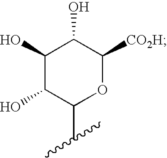
(c) —CH₂— substituted with one group selected from: —H, -methyl, —Oalkyl, —CH₂OH, —CH(CH₃)OH, —O(C═O)alkyl, —C(═O)Oalkyl, —(C═O)NH₂, —C(═O)alkyl or -phenyl-OC(═O)alkyl;
(f) —C(═O)alkyl optionally substituted with one or more groups independently selected from: —OH, —Oalkyl, —Oalkyl-phenyl or —OC(═O)alkyl;
(g) —C(═O)phenyl optionally substituted with one or more groups independently selected from: —Oalkyl, chloro or fluoro,
(h) —C(═O)(CH₂)₁₋₆C(═O)— terminating with —OH, —Oalkyl, —NH₂, —NHalkyl, —N(alkyl)₂, or pyrrolidinyl;
(hh)—C(═O)alkylOC(═O)alkyl- terminating with —Oalkyl;
(i) —C(═O—O)O(CH₂CH₂O—)₁₋₁₀ terminating with H or benzyl;
(j) —C(═O)Oalkyloptionally substituted with one or more —Oalkyl groups;
(k) —C(═O)Ophenyl optionally substituted with one or more chloro, fluoro groups;
(l) —C(═O—O)NH(CH₂CH₂O—)₁₋₁₀ terminating with —H;
(m) —C(═O)NH(C₁₋₂₀)alkyl optionally substituted with one or more groups independently selected from: —NH₂, —NHalkyl, —N(alkyl)₂, pyrrolidinyl, morpholinyl, —NHC(═O)alkyl, —OC(═O)alkenyl, —NHC(═O—O)phenyl or —C(═O)Oalkyl; and, wherein the phenyl portion of said —NHC(═O)phenyl may be optionally substituted with one or more groups independently selected from: alkyl, —OH, —Oalkyl, —NH₂, —NHalkyl, —N(alkyl)₂, halogen and nitrile;
(n) —C(═O)NHphenyl optionally substituted with one or more fluoro groups;
(o)—C(═O)NHCH₂CH₂NH(CH₂CH₂NH—)₀₋₃ terminating with —CH₂CH₂OH and —C(═O)phenyl; wherein the phenyl portion of said —C(═O)phenyl may be optionally substituted with one or more —OH groups;
(p) —C(═S)NH₂;
(u) —SO₂N(alkyl)₂;
(w) —P(═O)(OCH₂CH₃)₂; or
(x) L₅.

An embodiment of the present invention includes compounds of Formula (I) wherein
R⁴ is independently selected from: H, 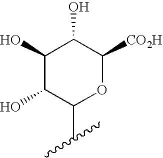
   provided that R⁶ is not 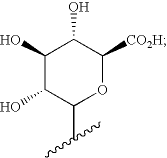
1-methoxy-1-oxo-ethyl, 1-methyl-ethoxy-carbonyl, 1-oxo-butoxy-methyl, 1-oxo-ethoxy-methyl, 1-oxo-ethyl, 1-oxo-propyl, 2-(1-oxo-ethoxy)-1-oxo-ethyl, 2-(2-methoxy-1-oxo-ethoxy)-1-oxo-ethyl, 2-(2-methyl-1-oxo-propoxy)-1-oxo-ethyl, 2-amino-2-oxo-ethyl, 2,2-dimethyl-1-oxo-propoxy-methyl, 2-ethoxy2-oxo-ethyl, 2-methoxy-2-oxo-ethyl, 2,6-difluoro-benzoyl, 2-[2-(2-hydroxy-ethoxy)-ethoxy]-ethoxy-carbonyl, 2-benzyloxy-1-oxo-ethyl, 2-benzyloxy-ethoxy-carbonyl, 2-chloro-phenoxy-carbonyl, 2-fluoro-benzoyl, 2-hydroxy-1-oxo-ethyl, 2-hydroxy-ethyl, 2-hydroxy-propyl, 2-methoxy-1-oxo-ethyl, 2-methoxy-ethoxy-carbonyl, 2-methyl-1-oxo-propyl, 2-oxo-propyl, 3-(N,N-diethyl amino)-1,3-dioxo-propyl, 3-1H-pyrrolidin-1-yl-1,3-dioxo-propyl, 3-ethoxy-1,3-dioxo-propyl, 3-1H-pyrrolidin-1-yl-1,3-dioxo-propyl, 4-(1-oxo-ethoxy)-benzyl, 4-amino-1,4-dioxo-n-butyl, 4-ethoxy-1,4-dioxo-n-butyl, 4-hydroxy-1,4-dioxo-n-butyl, 4-methoxy-1,4-dioxo-n-butyl, 4-chloro-benzoyl, 4-chloro-phenoxy-carbonyl, 4-fluoro-benzoyl, 4-fluoro-phenoxy-carbonyl, 4-methoxy-benzoyl, 5-(N-methyl-amino)-1,5-dioxo-pentyl, 5-methoxy-1,5-dioxo-pentyl, benzoyl, diethoxy-phosphinyl, ethoxy-carbonyl, methoxy-carbonyl, methoxy-methyl, methyl, N-(2-ethoxy-2-oxo-ethyl)-amino-carbonyl, N-(2-1H-pyrrolidin-1-yl-ethyl)-amino-carbonyl, N-(2-amino-ethyl)-amino-carbonyl, N-(2-morpholin-4-yl-ethyl)-amino-carbonyl, N-(3-ethoxy-3-oxo-propyl)-amino-carbonyl, N-(3-fluoro-phenyl)-amino-carbonyl, N-(pentadecyl)-amino-carbonyl, N,N-dimethyl-amino-sulfonyl, N-[2-(2-methenyl-1-oxo-propoxy)-ethyl]-amino-carbonyl, N-[2-(3-methyl-1-methoxy-1-oxo)-n-butyl]-amino-carbonyl, N-[2-(4-methyl-1-methoxy-1-oxo)-pentyl]-amino-carbonyl, N-[2-(N,N-dimethyl-amino)-ethyl]-amino-carbonyl, N-[2-(N-benzoyl-amino)-ethyl]-amino-carbonyl, N-[2-(N-methyl-amino)-ethyl]-amino-carbonyl, N-[2-[2-(2-hydroxy-ethoxy)-ethoxy]-ethyl]-amino-carbonyl, N-[2-[N-(1-oxo-ethyl)-amino]-ethyl]-amino-carbonyl, N-[2-[N-(2-hydroxy-benzoyl)-amino]-ethyl]-amino-carbonyl, N-[2-[N-(2-hydroxy-ethyl)-amino]-ethyl]-amino-carbonyl, N-[2-[N-(2-methyl-1-oxo-propyl)-amino]-ethyl]-amino-carbonyl, N-methyl-amino-carbonyl, N-methyl-amino-thiocarbonyl, and phenoxy-carbonyl, or L₅.

A preferred embodiment of the present invention includes compounds of Formula (I) wherein:
L₅ is selected from -M¹-K-J¹-Matrix or -M¹-K-J³-X-Matrix;
wherein:
M¹ is selected from —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄(C═O)O—, —(CH₂)₂₋₄S—, or —(CH₂)₂₋₄O—; provided that in L₅ the attachment of M¹ to K to J¹ or J³ is not through a N—O or O—N bond, a N—S or S—N bond, an O—S or S—O bond, or an O—O bond.

A preferred embodiment of the present invention includes compounds of Formula (I) wherein:
M¹ is selected from —C(═O)NH—, —(CH₂)₂₋₄NHC(═O)—, —(CH₂)₂₋₄(C═O)NH—, —(CH₂)₂₋₄NHC(═O)O—, —(CH₂)₂₋₄S—, or —(CH₂)₂₋₄O—.

An embodiment of the present invention includes compounds of Formulae (I) wherein:
L₃ is absent or is a linking group selected from the group consisting of alkylidyl, carbonyl, -alkylidyl-C(═O)—, or —C(═O)-alkylidyl-.

An embodiment of the present invention includes compounds of Formulae (I) wherein:
L₃ is absent or is a linking group selected from the group consisting of alkylidyl or carbonyl.

An embodiment of the present invention includes compounds of Formulae (I) wherein:
L₃ is absent or alkylidyl.

An embodiment of the present invention includes compounds of Formulae (I) wherein:
B is selected from the group consisting of aryl, heteroaryl, a nine to ten membered benzo-fused cycloalkyl, —CH(R⁹)aryl, and —CH(R⁹)heteroaryl; wherein the aromatic portion of said B is optionally substituted with R⁵; and wherein R⁹ is a substituent selected from hydrogen, C₁₋₅alkyl, or cycloalkyl; wherein said alkyl is optionally substituted with alkylamino, amino, cyano, dialkylamino, halogenated alkyl, halogenated alkyloxy, —SO₂alkyl, or hydroxy.

An embodiment of the present invention includes compounds of Formulae (I) wherein:
B is selected from the group consisting of aryl, heteroaryl, a nine to ten membered benzo-fused cycloalkyl, —CH(R⁹)aryl, and —CH(R⁹)heteroaryl; wherein the aromatic portion of said B is optionally substituted with R⁵; and wherein R⁹ is a substituent selected from hydrogen or C₁₋₅alkyl; wherein said alkyl is optionally substituted with alkylamino, amino, cyano, dialkylamino, or hydroxy.

An embodiment of the present invention includes compounds of Formulae (I) wherein:
B is selected from the group consisting of aryl, heteroaryl, or a nine to ten membered benzo-fused cycloalkyl; wherein the aromatic portion of said B is optionally substituted with R⁵.

An embodiment of the present invention includes compounds of Formulae (I) wherein:
B is selected from the group consisting of phenyl, naphthyl, pyridinyl, indanyl, or tetralinyl; wherein the aromatic portion of said B is optionally substituted with R⁵.

An embodiment of the present invention includes compounds of Formulae (I) wherein:
B is selected from phenyl or pyridinyl wherein the aromatic portion of said B is optionally substituted with R⁵.

An embodiment of the present invention includes compounds of Formulae (I) wherein:
R⁵ is independently selected from the group consisting of L₄, alkyl, alkyloxy, amino, —C(═O)NH₂, —C(═O)Oalkyl, —C(═O)OH, cyano, dialkylamino, halogen, halogenated alkyl, halogenated alkyloxy, —SO₂-alkyl, halogenated thioalkyl, hydroxy, hydroxy alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, —SO₂NH₂, thio, thioalkyl, 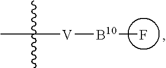
   and -V-B¹⁰-W-B²⁰.

An embodiment of the present invention includes compounds of Formulae (I) wherein:
R⁵ is selected from the group consisting of L₄, alkyl, alkyloxy, —C(═O)NH₂, —C(═O)Oalkyl, —C(═O)OH, cyano, dialkylamino, halogen, halogenated alkyl, halogenated alkyloxy, halogenated thioalkyl, hydroxy, hydroxy alkyl, nitro, —SO₂alkyl, —SO₂NH₂, thioalkyl, 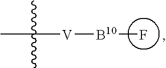
   and -V-B¹⁰-W-B²⁰.

An embodiment of the present invention includes compounds of Formulae (I) wherein:
R⁵ is independently selected from the group consisting of: L₄, methyl, ethyl, isopropyl, t-butyl, methoxy, ethoxy, —C(═O)NH₂, —C(═O)Omethyl, —C(═O)Oethyl, —C(═O)OH, cyano, dimethyl-amino, bromo, chloro, fluoro, trifluoromethyl, trifluoromethoxy, thio-trifluoromethyl, hydroxy, hydroxymethyl, hydroxyethyl, nitro, —SO₂NH₂, thiomethyl 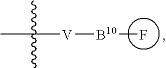
   and -V-B¹⁰-W-B²⁰.

An embodiment of the present invention includes compounds of Formula (1):
wherein V and W are each independently absent or selected from the group consisting of: —C(═O), —C(═O)N(alkyl)-, —C(═O)NH—, —C(═O)O—, —NH—, —NHC(═O)—, —NHC(═O)NH—, —NHC(═O)O—, —NHSO₂—, —O—, —OC(═O), —OC(═O)NH—, —OC(═O)O—, —S—, —SO—, —SO₂—, and —SO₂NH—.

An embodiment of the present invention includes compounds of Formula (I):
wherein V and W are each independently absent or selected from the group consisting of: —C(═O), —C(═O)N(alkyl)-, —C(═O)NH—, —C(═O)O—, —NH—, —O— and —SO₂—.

An embodiment of the present invention includes compounds of Formula (I) wherein V is absent or selected from the group consisting of: —C(═O), —C(═O)N(alkyl)-, —C(═O)NH—, —C(═O)O—, —NH—, —O— and —SO₂—.

An embodiment of the present invention includes compounds of Formula (I) wherein V is absent or selected from the group consisting of: —C(═O)NH—, —C(═O)O—, —NH—, —O— and —SO₂—.

An embodiment of the present invention includes compounds of Formula (I) wherein W is absent.

An embodiment of the present invention includes compounds of Formula (I) wherein:
B¹⁰ is absent or alkyl; and
   wherein when B¹⁰ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl.

An embodiment of the present invention includes compounds of Formula (I) wherein:
B¹⁰ is absent or alkyl; and
   wherein when B¹⁰ is alkyl, the alkyl may be optionally substituted with one or more groups independently selected from: alkoxy, alkylamino, amino, dialkylamino, halogen, halogenated alkoxy, hydroxy —NHC(═O)NH₂, —NHSO₂alkyl, —SO₂alkyl or thioalkyl.

An embodiment of the present invention includes compounds of Formula (I) wherein:
B¹⁰ is absent or selected from methyl or ethyl, wherein methyl or ethyl are optionally substituted with one or more groups independently selected from dialkylamino or hydroxy.

An embodiment of the present invention includes compounds of Formula (I) wherein:
B¹⁰ is absent or selected from methyl or ethyl, wherein methyl or ethyl are optionally substituted with one or more groups independently selected from dimethyl-amino or hydroxy.

An embodiment of the present invention includes compounds of Formula (I) wherein:
B²⁰ is absent or selected from alkyl, alkenyl, or H;
   wherein, when B²⁰ is alkyl or alkenyl, the alkyl or alkenyl group may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl.

An embodiment of the present invention includes compounds of Formula (I) wherein:
B²⁰ is absent or selected from alkyl, alkenyl, or H;
   wherein, when B²⁰ is alkyl or alkenyl, the alkyl or alkenyl group may be optionally substituted with one or more groups independently selected from: alkoxy, alkylamino, amino, dialkylamino, halogen, halogenated alkoxy, hydroxy, —NHC(═O)NH₂, —NHSO₂alkyl or thioalkyl.

An embodiment of the present invention includes compounds of Formula (I) wherein:
B²⁰ is absent or H.

An embodiment of the present invention includes compounds of Formula (I) wherein: 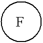
is selected from the group consisting of aryl, cycloalkyl, partially unsaturated carbocycle, heteroaryl and heterocycloalkyl optionally substituted with one or more substituents independently selected from alkoxy, alkyl, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl.

An embodiment of the present invention includes compounds of Formula (I) wherein: 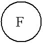
is selected from the group consisting of phenyl, imidazolyl, pyridinyl, pyrimidinyl, pyrrolidinyl, morpholinyl, piperazinyl and piperidinyl optionally substituted with one or more substituents independently selected from alkoxy, alkyl, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl.

An embodiment of the present invention includes compounds of Formula (I) wherein: 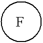
is selected from the group consisting of phenyl, imidazolyl, pyridinyl, pyrimidinyl, pyrrolidinyl, morpholinyl, piperazinyl and piperidinyl optionally substituted with one or more substituents independently selected from alkoxy, alkyl, alkylamino, amino, dialkylamino, halogen, halogenated alkoxy, halogenated alkyl, heteroaryl, hydroxy, hydroxy alkyl, —NHC(═O)NH₂, —NHSO₂alkyl, nitro or —SO₂alkyl.

An embodiment of the present invention includes compounds of Formula (I) wherein: 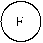
is selected from the group consisting of phenyl, imidazolyl, pyridinyl, pyrimidinyl, pyrrolidinyl, morpholinyl, piperazinyl and piperidinyl optionally substituted with one or more substituents independently selected from methoxy, ethoxy, methyl, ethyl, bromo, chloro, fluoro, trifluoromethyl, pyridinyl, hydroxy or hydroxymethyl.

A preferred embodiment of the present invention includes compounds of Formula (I) wherein L₄ is -M-K-J¹-Matrix or -M-K-J³-X-Matrix and L₅ is -M¹-K-J¹-Matrix or -M¹-K-J³-X-Matrix and L₄ and L₅ are of a length and degree of flexibility that permits binding between the precursor compound and the target proteins.

In another preferred embodiment, the modification of precursor compounds of Formula (I) by incorporation of linker L₄ or L₅ does not diminish the biological activity of the precursor compound.

An embodiment of the present invention includes compounds of Formula (I) wherein: Matrix comprises a solid support material.

A preferred embodiment of the present invention includes compounds of Formula (I) wherein: Matrix comprises a solid support material selected from the group consisting of: gel, cellulose, glass, plastic material, beads, and plates.

A preferred embodiment of the present invention includes compounds of Formula (I) wherein: Matrix is selected from the group consisting of: Ciphergen PS10 chip; Ciphergen PS20 chip; Reacti-Gel; UltraLink; UltraLink DADPA, PharmaLink, AminoLink; CarboLink; SulfoLink; MagnaBind bead; and UltraLink maleimide.

In another preferred embodiment, the present invention is further directed to compounds of Formula (1-AA) 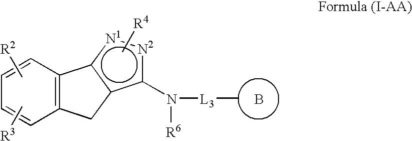
wherein
R² is independently selected from the group consisting of: alkoxy, alkyl, amino, cyano, dialkylamino, halogen, halogenated alkyl, halogenated alkyloxy, hydroxylated alkyloxy, hydroxy, —NHC(═O)alkyl, and L₄;
R³ is independently selected from the group consisting of 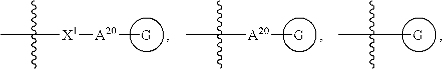
   -X¹-A²⁰-A²¹ and -X¹-A²¹, —CO₂H, —C(═O)Oalkyl, —C(═O)NH₂, —C(═O)NH(alkyl), —C(═O)N(alkyl)₂, and L₄;
   wherein X¹ and Y¹ are each independently absent or selected from the group consisting of: —C(═O)NH—, —C(═O)O—, —NH—, —NHC(═O)—, —NHC(═O)NH, —NHC(═O)O—, —NHSO₂—, —O—, —OC(═O), —OC(═O)NH—, —OC(═O)O—, —S—, —SO—, —SO₂— and —SO₂NH—;
A²⁰ is absent or alkyl;
A²¹ is selected from alkyl, alkenyl, or H;
wherein when A²⁰ or A²¹ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 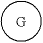
   is selected from the group consisting of aryl, heteroaryl and heterocycloalkyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂;
L₄ is selected from -M-K-J¹-Matrix or -M-K-J³-X-Matrix;
   wherein M is selected from —C(═O)NH—, —O—, —NHC(═O)O—, —S—, or —SS—;
   and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)N H(CH₂)ᵣ₂]ₙ(CH₂)ₚ—,
—(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)N H(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—,
—(CH₂)ₘ(OCH₂CH₂)ₙSS(CH₂)ₚ—; and
   wherein J¹ is selected from —C(═O)NH—, —O—, —NHC(═O)O—, —S—, —SS—, —SCH₂(CHOH)—, —CH═NNH—, 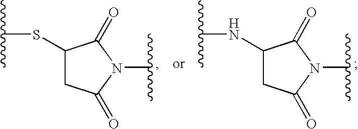
      and
   wherein J³ is selected from the group consisting of —NH—, —O—, —NHNH—, —NHNH═CH—,
—NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHNH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C═O)NHNH(C═O)NH—, and —NH(CH₂)₆—NH(C═O)CH₂CH₂SS—;
   provided that in L₄ the attachment of M to K to J¹ or J³ is not through a N—O or O—N bond, a N—S or S—N bond, an O—S or S—O bond, or an O—O bond;
X is selected from biotin/avidin, biotin/streptavidin, iminobiotin/avidin, or iminobiotin/streptavidin, biotin/NeutrAvidin™, or iminobiotin/NeutrAvidin™;
R⁴ is independently selected from:
(a) H;
(b) 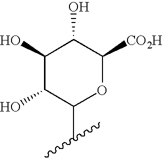
   provided that R⁶ is not 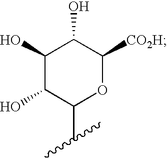
(c) —CH₂— substituted with one group selected from: —H, -methyl, —Oalkyl, —CH₂OH, —CH(CH₃)OH, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(f) —C(═O)alkyl optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)(CH₂)₁₋₃aryl or —C(═O)aryl, wherein said —C(═O)aryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh) —C(═O)alkylOC(═O)alkyl- terminating with —OH, —Oalkyl, —NH₂, —NHalkyl or —N(alkyl)₂;
(i) —C(═O)O(CH₂CH₂O—)₁₋₁₀ terminating with H, methyl, ethyl, or benzyl;
(j) —C(═O)Oalkyloptionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)O(CH₂)₁₋₃aryl or —C(═O)Oaryl, wherein said —C(═O)Oaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH₂, —C(═O)NH(C₁₋₂₀)alkyl, or —C(═O)N(C₁₋₂₀alkyl)₂, wherein said —C(═O)NH(C₁₋₂₀)alkyl, may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NHalkyl, —N(alkyl)₂, halogen or nitrile;
(n) —C(═O)NH(CH₂)₁₋₃aryl or —C(═O)NHaryl, wherein said —C(═O)NHaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(u) —SO₂N(alkyl)₂;
(w) —P(═O)(OCH₂CH₃)₂; or
(x) L₅.
   L₅ is selected from -M¹-K-J¹-Matrix or -M¹-K-J³-X-Matrix;
   wherein:
   M¹ is selected from —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄(C═O)O—, —(CH₂)₂₋₄S—, or —(CH₂)₂₋₄O—;
      provided that in L₅ the attachment of M¹ to K to J¹ or J³ is not through a N—O or O—N bond, a N—S or S—N bond, an O—S or S—O bond, or an O—O bond; and
L₃ is absent or is a linking group selected from the group consisting of alkylidyl, carbonyl, -alkylidyl-C(═O)—, or —C(═O)-alkylidyl-;
B is selected from the group consisting of aryl, heteroaryl, a nine to ten membered benzo-fused cycloalkyl, —CH(R⁹)aryl, and —CH(R⁹)heteroaryl; wherein the aromatic portion of said B is optionally substituted with R⁵; and wherein R⁹ is a substituent selected from hydrogen, C₁₋₅alkyl, or cycloalkyl; wherein said alkyl is optionally substituted with alkylamino, amino, cyano, dialkylamino, halogenated alkyl, halogenated alkyloxy, —SO₂alkyl, or hydroxy;
R⁵ is selected from the group consisting of L₄, alkyl, alkyloxy, —C(═O)NH₂, —C(═O)Oalkyl, —C(═O)OH, cyano, dialkylamino, halogen, halogenated alkyl, halogenated alkyloxy, halogenated thioalkyl, hydroxy, hydroxy alkyl, nitro, —SO₂alkyl, —SO₂NH₂, thioalkyl, 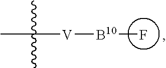
   and -V-B¹⁰-W-B²⁰;
wherein V and W are each independently absent or selected from the group consisting of: —C(═O), —C(═O)N(alkyl)-, —C(═O)NH—, —C(═O)O—, —NH—, —NHC(═O)—, —NHC(═O)NH—, —NHC(═O)O—, —NHSO₂—, —O—, —OC═O), —OC(═O)NH—, —OC(═O)O—, —S—, —SO—, —SO₂—, and —SO₂NH—;
B¹⁰ is absent or alkyl;
B²⁰ is absent or selected from alkyl, alkenyl, or H;
wherein when B¹⁰ or B²⁰ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 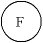
   is selected from the group consisting of aryl, cycloalkyl, partially unsaturated carbocycle, heteroaryl and heterocycloalkyl optionally substituted with one or more substituents independently selected from alkoxy, alkyl, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl;
      provided that at least one, and no more than one, L₄ or L₅ is present;
      or an optical isomer, enantiomer, diastereomer, racemate, or pharmaceutically acceptable salt thereof.

In another preferred embodiment, the present invention is further directed to compounds of Formula (1-BB): 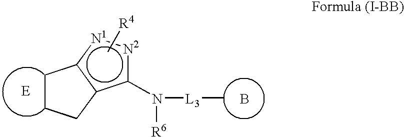
wherein: 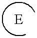
is selected from the group consisting of Formulae A-1, A-2 and A-3: 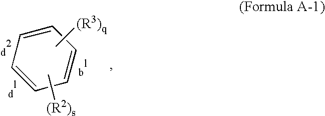
wherein Formula A-1 is attached on the b¹ side of Formula A-1 to the R¹ substituted ring of formula (I) and optionally substituted with one substituent selected from the group consisting of Formulae A-1-a, A-1-b and A-1-c: 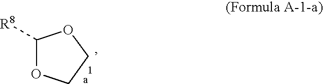
wherein Formula A-1-a is attached on the a¹ side to adjacent carbons on the d¹ or d² side of Formula A-1; 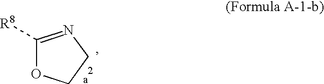
wherein Formula A-1-b is attached on the a² side to adjacent carbons on the d¹ or d² side of Formula A-1; and 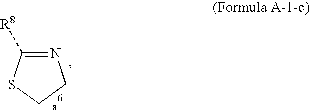
wherein Formula A-1-c is attached on the a⁶ side to adjacent carbons on the d¹ or d² side of Formula A-1;
wherein R⁸ is hydrogen, lower alkyl, or L₄; 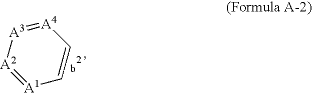
wherein Formula A-2 is attached on the b² side of Formula A-2 to the R¹ substituted ring of formulae (I), and one or two of A¹, A², A³, A⁴ are —N—; the remainder being —C— substituted with H or alkoxy, wherein the alkoxy may be optionally further substituted with alkoxy on a terminal carbon or up to 3 halogen atoms on a terminal carbon; and 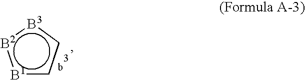
wherein Formula A-3 is attached on the b³ side of Formula A-3 to the R¹ substituted ring of formulae (I), and B¹, B² and B³ are independently (i) —CH— optionally substituted with C₁₋₄alkyl, aryl, alkoxy, or halogen, (ii) —S—, (iii) —O— or (iv) —N—; provided that no more than one of B¹, B² or B³ is —S— or —O—, and, provided that when one of B¹, B² or B³ is —S— or —O—, then the adjacent ring members are not —S— or —O—;
R² is independently selected from the group consisting of: alkoxy, alkyl, amino, cyano, dialkylamino, halogen, halogenated alkyl, halogenated alkyloxy, hydroxylated alkyloxy, hydroxy, —NHC(═O)alkyl, and L₄;
R³ is independently selected from the group consisting of 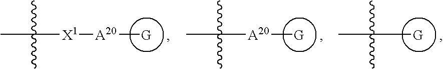
   —X¹-A²⁰-A²¹ and —X¹-A²¹, —CO₂H, —C(═O)Oalkyl, —C(═O)NH₂, —C(═O)NH(alkyl), —C(═O)N(alkyl)₂, and L₄;
wherein X¹ and Y¹ are each independently absent or selected from the group consisting of: —C(═O)NH—, —C(═O)O—, —NH—, —NHC(═O)—, —NHC(═O)NH, —NHC(═O)O—, —NHSO₂—, —O—, —OC(═O), —OC(═O)NH—, —OC(═O)O—, —S—, —SO—, —SO₂— and —SO₂NH—;
A²⁰ is absent or alkyl;
A²¹ is selected from alkyl, alkenyl, or H;
wherein when A²⁰ or A²¹ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 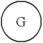
   is selected from the group consisting of aryl, heteroaryl and heterocycloalkyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂;
L₄ is selected from -M-K-J¹-Matrix or -M-K-J³-X-Matrix;
   wherein M is selected from —C(═O)NH—, —O—, —NHC(═O)O—, —S—, or —SS—;
   and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)NH(CH₂)ᵣ₂]ₙ(CH₂)ₚ—,
—(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—, or
—(CH₂)ₘ(OCH₂CH₂)ₙSS(CH₂)ₚ—; and
   wherein J¹ is selected from —C(═O)NH—, —O—, —NHC(═O)O—, —S—, —SS—, —SCH₂(CHOH)—, —CH═NNH—, 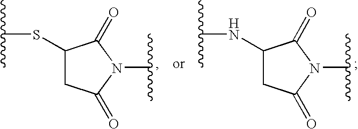
      and
   wherein J³ is selected from the group consisting of —NH—, —O—, —NHNH—, —NHNH═CH—,
—NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—N H NH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C═O)NHNH(C═O)NH—, and —NH(CH₂)₆—NH(C═O)CH₂CH₂SS—;
   provided that in L₄ the attachment of M to K to J¹ or J³ is not through a N—O or O—N bond, a N—S or S—N bond, an O—S or S—O bond, or an O—O bond;
X is selected from biotin/avidin, biotin/streptavidin, iminobiotin/avidin, or iminobiotin/streptavidin, biotin/NeutrAvidin™, or iminobiotin/NeutrAvidin™;
R⁴ is independently selected from:
(a) H;
(b) 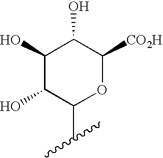
   provided that R⁶ is not 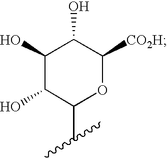
(c) alkyl;
(d) —C(═O)alkylOH;
(e) —C(═O)(CH₂)₂Oalkyl; or
(f) L₅;
   L₅ is selected from -M¹-K-J¹-Matrix or -M¹-K-J³-X-Matrix;
   wherein:
   M¹ is selected from —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄(C═O)O—, —(CH₂)₂₋₄S—, or —(CH₂)₂₋₄O—;
      provided that in L₅ the attachment of M¹ to K to J¹ or J³ is not through a N—O or O—N bond, a N—S or S—N bond, an O—S or S—O bond, or an O—O bond;
L₃ is absent or is a linking group selected from the group consisting of alkylidyl, carbonyl, —(C₁₋₄)alkylidyl-C(═O)—, or —C(═O)—(C₁₋₄)alkylidyl-;
B is selected from the group consisting of aryl, heteroaryl, a nine to ten membered benzo-fused cycloalkyl, —CH(R⁹)aryl, and —CH(R⁹)heteroaryl; wherein the aromatic portion of said B is optionally substituted with R⁵; and wherein R⁹ is a substituent selected from hydrogen, C₁₋₅alkyl, or cycloalkyl; wherein said alkyl is optionally substituted with alkylamino, amino, cyano, dialkylamino, halogenated alkyl, halogenated alkyloxy, —SO₂alkyl, or hydroxy;
R⁵ is selected from the group consisting of L₄, alkyl, alkyloxy, —C(═O)NH₂, —C(═O)Oalkyl, —C(═O)OH, cyano, dialkylamino, halogen, halogenated alkyl, halogenated alkyloxy, halogenated thioalkyl, hydroxy, hydroxy alkyl, nitro, —SO₂alkyl, —SO₂NH₂, thioalkyl, 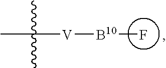
   and -V-B¹⁰-W-B²⁰;
wherein V and W are each independently absent or selected from the group consisting of: —C(═O), —C(═O)N(alkyl)-, —C(═O)NH—, —C(═O)O—, —NH—, —NHC(═O)—, —NHC(═O)NH—, —NHC(═O)O—, —NHSO₂—, —O—, —OC(═O), —OC(═O)NH—, —OC(═O)O—, —S—, —SO—, —SO₂—, and —SO₂NH—;
B¹⁰ is absent or alkyl;
B²⁰ is absent or selected from alkyl, alkenyl, or H;
wherein when B¹⁰ or B²⁰ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 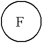
   is selected from the group consisting of aryl, cycloalkyl, partially unsaturated carbocycle, heteroaryl and heterocycloalkyl optionally substituted with one or more substituents independently selected from alkoxy, alkyl, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl;
      provided that at least one, and no more than one, L₄ or L₅ is present;
      or an optical isomer, enantiomer, diastereomer, racemate, or pharmaceutically acceptable salt thereof.

In a preferred embodiment, the present invention is further directed to compounds of Formula (1-CC): 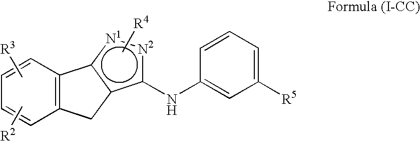
wherein:
R² is independently selected from the group consisting of: alkoxy, alkyl, amino, cyano, dialkylamino, halogen, halogenated alkyl, halogenated alkyloxy, hydroxylated alkyloxy, hydroxy, —NHC(═O)alkyl, and L₄;
R³ is independently selected from the group consisting of 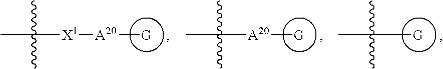
   -X¹-A²⁰-A²¹ and -X¹-A²¹, —CO₂H, —C(═O)Oalkyl, —C(═O)NH₂, —C(═O)NH(alkyl), —C(═O)N(alkyl)₂, and L₄;
wherein X¹ and Y¹ are each independently absent or selected from the group consisting of: —C(═O)NH—, —C(═O)O—, —NH—, —NHC(═O)—, —NHC(═O)NH, —NHC(═O)O—, —NHSO₂—, —O—, —OC(═O), —OC(═O)NH—, —OC(═O)O—, —S—, —SO—, —SO₂— and —SO₂NH—;
A²⁰ is absent or alkyl;
A²¹ is selected from alkyl, alkenyl, or H;
wherein when A²⁰ or A²¹ is alkyl, the alkyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 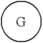
   is selected from the group consisting of aryl, heteroaryl and heterocycloalkyl optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂;
R⁵ is selected from the group consisting of L₄, alkyl, alkyloxy, —C(═O)NH₂, —C(═O)Oalkyl, —C(═O)OH, cyano, dialkylamino, halogen, halogenated alkyl, halogenated alkyloxy, halogenated thioalkyl, hydroxy, hydroxy alkyl, nitro, —SO₂alkyl, —SO₂NH₂, and thioalkyl;
L₄ is selected from -M-K-J¹-Matrix or -M-K-J³-X-Matrix;
   wherein M is selected from —C(═O)NH—, —O—, —NHC(═O)O—, —S—, or —SS—;
   and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C=o)NH(CH₂)ᵣ₂]ₙ(CH₂)ₚ—,
—(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—, or
—(CH₂)ₘ(OCH₂CH₂)ₙSS(CH₂)ₚ—; and
   wherein J¹ is selected from —C(═O)NH—, —(C═O)O—, —NHC(═O)—, —NH(C═O)O—, —O(C═O)NH—, —O(C═O)—, —O—, —NH—, —(CH₂)₁₋₃O—, —SS—, —S—, —SCH₂(CHOH)—, 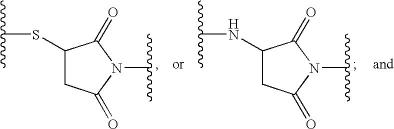
   wherein J³ is selected from the group consisting of —NH—, —O—, —NHNH—, —NHNH═CH—, —NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHN H—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)N H N H(C═O)O—, —NH—(CH₂)₂—SS—(CH₂)₂(C═O)NHNH(C═O)N H—, and —NH(CH₂)₆—NH(C═O)CH₂CH₂SS—;
      provided that in L₄ the attachment of M to K to J¹ or J³ is not through a N—O or O—N bond, a N—S or S—N bond, an O—S or S—O bond, or an O—O bond;
X is selected from biotin/avidin, biotin/streptavidin, iminobiotin/avidin, or iminobiotin/streptavidin, biotin/NeutrAvidin™, or iminobiotin/NeutrAvidin™;
R⁴ is independently selected from:
(a) H;
(b) 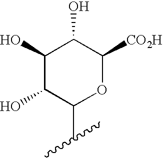
   provided that R⁶ is not 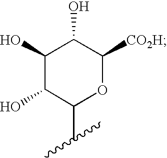
(c) alkyl;
(d) —C(═O)alkylOH;
(e) —C(═O)(CH₂)₂Oalkyl; or
(f) L₅;
L₅ is selected from -M¹-K-J¹-Matrix or -M¹-K-J³-X-Matrix;
   wherein:
   M¹ is selected from —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄(C═O)O—, —(CH₂)₂₋₄S—, or —(CH₂)₂₋₄O—;
      provided that in L₅ the attachment of M¹ to K to J¹ or J³ is not through a N—O or O—N bond, a N—S or S—N bond, an O—S or S—O bond, or an O—O bond; and
      provided that at least one, and no more than one, 4 or L₅ is present;
      or an optical isomer, enantiomer, diastereomer, racemate, or pharmaceutically acceptable salt thereof.

In a preferred embodiment, the present invention is further directed to a compound of Formula (I-DD): 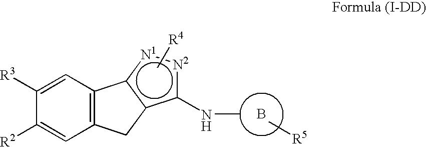
wherein R², R³, R⁴ B and R⁵ are as defined in the chart below.
Abbreviations:
Av-PS10=Avidin coated Ciphergen PS10 chip
Av-UL=UltraLink with avidin
Neut-UL=UltraLink with NeutrAvidin™
Strep-UL=UltraLink with Streptavidin
AL=AminoLink
CL=CarboLink
Imb=Iminobiotin
SL=SulfoLink
Strep-MB=MagnaBind bead with Streptavidin
UL-DADPA=UltraLink activated
   with diaminodipropylamine
UL-M=UltraLink maleimide
GF-2000=Reacti-Gel

PS10 or PS20=PS10 or PS20 Protein Chips

| | | | | | | L₄ or L₅ = -M⁽¹⁾-K-J^{1or3} -(X)-Matrix | | | |
|---|---|---|---|---|---|---|---|---|---|
| | | | | | | | | | -(X)- |
| Cpd | R² | R³ | R⁴ | B | R⁵ | M or M¹ | K | J^{1/3} | Matrix |
| 1 | OMe | OMe | H | phenyl | L₄ | —C(═O)NH— | —(CH₂)₂(OCH₂CH₂)₃— | —NH— | -Biotin- |
| | | | | | | | | | Av-PS10 |
| 2 | L₄ | OMe | H | phenyl | F | —O— | —(CH₂)₂(OCH₂CH₂)₂— | —NHC(═O)O— | GF2000 |
| 3 | OEt | L₄ | H | phenyl | OEt | —O— | —(CH₂)₂(OCH₂CH₂)(OCH₂)— | —NHC(═O)O— | PS10 |
| 4 | OMe | OMe | L₅ | phenyl | F | —(CH₂)₂O— | —(CH₂)₂(OCH₂CH₂)— | —NHC(═O)O— | PS10 |
| 5 | OMe | OMe | H | phenyl | L₄ | —O— | —(CH₂)₂(OCH₂CH₂)₂— | —NHC(═O)O— | PS10 |
| 6 | L₄ | OMe | H | phenyl | F | —O— | —(CH₂)₂(OCH₂CH₂)₂— | —NHC(═O)O— | PS10 |
| 7 | L₄ | OMe | H | phenyl | F | —O— | —(CH₂)₂(OCH₂CH₂)₂— | —NHC(═O)O— | GF2000 |
| 8 | L₄ | OMe | H | phenyl | F | —O— | —(CH₂)₂(OCH₂CH₂)₃— | —NHC(═O)O— | GF2000 |
| 9 | L₄ | OMe | H | phenyl | F | —O— | —(CH₂)₂(OCH₂CH₂)— | —NHC(═O)O— | GF2000 |
| 10 | L₄ | OMe | H | phenyl | F | —O— | —(CH₂)₂(OCH₂CH₂)₂— | 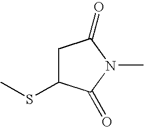 | UL-M |
| 11 | L₄ | OMe | H | phenyl | F | —O— | —(CH₂)₂(OCH₂CH₂)(OCH₂)— | —(C═O)NH— | UL-DADPA |
| 11a | L₄ | OMe | H | Phenyl | F | —O— | —(CH₂)₂(OCH₂CH₂)(OCH₂)— | —CH(═N)NH— | UL-H |
| 12 | L₄ | OMe | H | phenyl | F | —O— | —(CH₂)₂(OCH₂CH₂)(OCH₂)— | —(C═O)O— | UL-DADPA |
| 13 | L₄ | OMe | H | phenyl | F | —O— | —(CH₂)₂(OCH₂CH₂)₂— | —OC(═O)— | CL |
| 14 | L₄ | H | H | phenyl | F | —O— | —(CH₂)₂(OCH₂CH₂)₂— | —NHC(═O)— | CL |
| 15 | L₄ | OMe | H | phenyl | OMe | —O— | —(CH₂)₂(OCH₂CH₂)₂— | 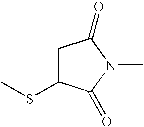 | UL-M |
| 16 | L₄ | OMe | H | phenyl | Br | —O— | —(CH₂)₂(OCH₂CH₂)₂— | —NHC(═O)O— | GF2000 |
| 17 | L₄ | OMe | H | phenyl | —CO₂Me | —O— | —(CH₂)₂(OCH₂CH₂)₂— | —NH— | AL |
| 18 | L₄ | OMe | H | phenyl | F | —O— | —(CH₂CH₂SCH₂CH₂O(CH₂)₂— | —NH— | AL |
| 19 | L₄ | OMe | H | phenyl | OMe | —O— | —(CH₂)₂(OCH₂CH₂)₂— | —SCH₂CH(OH)— | PS20 |
| 20 | L₄ | OMe | H | pyridinyl | Cl | —O— | —(CH₂)₂(OCH₂CH₂)₃— | —NHC(═O)O— | GF2000 |
| 21 | L₄ | OMe | H | phenyl | F | —O— | —(CH₂)₃NH(C═O)(CH₂)₃— | —NHC(═O)O— | GF2000 |
| 22 | L₄ | OMe | H | phenyl | F | —O— | —(CH₂)₃NHC(═O)CH₂— | 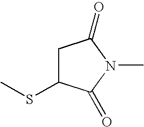 | UL-M |
| 23 | L₄ | OMe | H | phenyl | F | —O— | —(CH₂)₃NHC(═O)CH₂SS(CH₂)₄— | —NHC(═O)O— | PS10 |
| 24 | L₄ | OMe | H | phenyl | F | —O— | —(CH₂)₃(C═O)NH(CH₂)₂— | —NHC(═O)O— | PS10 |
| 25 | L₄ | OMe | H | phenyl | F | —O— | —(CH₂)₃(C(═O)NH(CH₂)₄NHC(═O)(CH₂)₃— | —NHC(═O)O— | GF2000 |
| 26 | L₄ | OMe | H | pyridinyl | Cl | —O— | —(CH₂)₃NHC(═O)(CH₂)— | 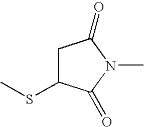 | UL-M |
| 27 | L₄ | OMe | H | pyridinyl | Cl | —O— | —(CH₂)₃NHC(═O)CH₂SS(CH₂)₄— | —NHC(═O)O— | GF2000 |
| 28 | L₄ | OMe | H | pyridinyl | Cl | —O— | —(CH₂)₃C(═O)NH(CH₂)₄NHC(═O)(CH₂)₃— | —NHC(═O)O— | GF2000 |
| 29 | L₄ | OMe | H | pyridinyl | Cl | —O— | —(CH₂)₃NHC(═O)(CH₂)₃— | —NHC(═O)O— | GF2000 |
| 30 | OEt | L₄ | H | phenyl | F | —O— | —(CH₂)₂(OCH₂CH₂)— | —NHC(═O)O— | GF2000 |
| 31 | OMe | L₄ | H | phenyl | F | —O— | —(CH₂)₂(OCH₂CH₂)₂— | 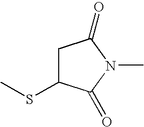 | UL-M |
| 32 | OMe | L₄ | H | phenyl | F | —O— | —(CH₂)₂OCH₂CH₂)₂— | —SCH₂CH(OH)— | PS20 |
| 33 | OMe | L₄ | H | phenyl | F | —O— | —(CH₂)₃(OCH₂CH₂)(OCH₂)— | —C(═O)NH— | UL-DADPA |
| 34 | OMe | L₄ | H | phenyl | F | —O— | —(CH₂)₂(OCH₂CH₂)₂(OCH₂)— | —C(═O)O— | SL |
| 35 | OEt | L₄ | H | phenyl | F | —O— | —(CH₂)₂(OCH₂CH₂)₂— | —OC(═O)— | CL |
| 36 | H | L₄ | H | phenyl | F | —O— | —(CH₂)₂(OCH₂CH₂)₂— | —NHC(═O)— | CL |
| 37 | OMe | L₄ | H | phenyl | F | —O— | —(CH₂CH₂SCH₂CH₂O)(CH₂)₂— | —NH— | AL |
| 38 | OMe | L₄ | H | phenyl | OMe | —O— | —(CH₂)₂(OCH₂CH₂)₂— | 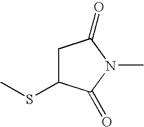 | UL-M |
| 39 | OMe | L₄ | H | phenyl | Br | —O— | —(CH₂)₂(OCH₂CH₂)₂— | —NHC(═O)O— | GF2000 |
| 40 | OMe | L₄ | H | phenyl | CO₂Me | —O— | —(CH₂)₂(OCH₂CH₂)₂— | —NH— | AL |
| 41 | OEt | L₄ | H | pyridin | Cl | —O— | —(CH₂)₂(OCH₂CH₂)— | —NHC(O)O— | PS10 |
| 42 | OMe | OMe | H | phenyl | L₄ | —C(═O)NH— | —(CH₂)₃(OCH₂CH₂)₂(CH₂)₂— | —NHC(═O)O— | PS10 |
| 43 | OMe | OMe | H | phenyl | L₄ | —C(═O)NH— | —(CH₂)₃(OCH₂CH₂)₂— | 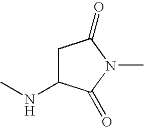 | UL-M |
| 44 | OMe | OMe | H | phenyl | L₄ | —C(═O)NH— | —(CH₂)₅C(═O)NH(CH₂)₄— | —NHC(═O)— | CL |
| 45 | OMe | OMe | H | phenyl | L₄ | —O— | —(CH₂)₂(OCH₂CH₂)₂(CH₂)₂— | —NH— | AL |
| 46 | OMe | OMe | H | phenyl | L₄ | —O— | —(CH₂)₂(OCH₂CH₂)₂(CH₂)₂— | —NHC(═O)— | PS10 |
| 47 | OMe | OMe | H | phenyl | L₄ | —O— | —(CH₂)₂(OCH₂CH₂)₂C(═O)NH(CH₂)₄— | —NHC(═O)— | CL |
| 48 | OMe | OMe | H | phenyl | L₄ | —C(═O)NH— | —(CH₂)₂(OCH₂CH₂)C(═O)NH(CH₂)₄— | —NHC(═O)— | CL |
| 49 | OEt | —O(CH₂)₃OH | H | phenyl | L₄ | —O— | —(CH₂)₂(OCH₂CH₂)₂C(═O)NH(CH₂)₄— | —NHC(═O)— | CL |
| 50 | OEt | —O(CH₂)₃OH | H | phenyl | L₄ | —O— | —(CH₂)₂(OCH₂CH₂)₂— | 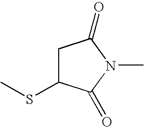 | UL-M |
| 51 | OEt | —O(CH₂)₃OH | H | phenyl | L₄ | —O— | —(CH₂)₂(OCH₂CH₂)₂— | —SCH₂CH(OH)— | PS20 |
| 52 | OMe | OMe | L₅ | phenyl | F | —(CH₂)₂O— | —(CH₂)₂(OCH₂CH₂)₃— | —NHC(═O)O— | GF2000 |
| 53 | OMe | OMe | L₅ | phenyl | F | —(CH₂)₂O— | —(CH₂)₂— | —NHC(═O)— | CL |
| 54 | OMe | OMe | L₅ | phenyl | F | —(CH₂)₂O— | —(CH₂)₂(OCH₂CH₂)— | 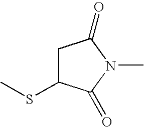 | UL-M |
| 55 | OMe | OMe | L₅ | phenyl | F | —(CH₂)₂O— | —(CH₂)₂(OCH₂CH₂)— | —NH— | -biotin- |
| | | | | | | | | | Strep-MB- |
| 56 | OMe | OMe | L₅ | phenyl | F | —(CH₂)₂O— | —(CH₂)₂(OCH₂CH₂)— | —OC(═O)— | CL |
| 57 | OMe | Ome | L₅ | phenyl | F | —(CH₂)₂O— | —(CH₂)₂(OCH₂CH₂)— | —C(O)NH— | UL-DADPA |
| 58 | OMe | OMe | L₅ | phenyl | F | —(CH₂)₂O— | —(CH₂)₂NHC(═O)(CH₂)— | 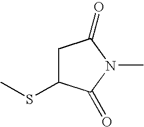 | UL-M |
| 59 | OMe | OMe | L₅ | pyridinyl | Cl | —(CH₂)₂O— | —(CH₂)₂(OCH₂CH₂)— | —C(═O)NH— | UL-DADPA |
| 60 | OMe | OMe | L₅ | pyridinyl | Cl | —(CH₂)₂O— | —(CH₂)₂NHC(═O)CH₂— | 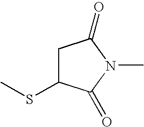 | UL-M |
| 61 | OMe | OMe | L₅ | phenyl | F | —(CH₂)₂O— | —(CH₂)₃C(═O)NH(CH₂)₃NHC(═O)(CH₂)₄— | —NHC(═O)— | CL |
| 62 | H | OMe | L₅ | phenyl | F | —(CH₂)₂O— | —(CH₂)₂(OCH₂CH₂)₂— | —NH— | AL |
| 63 | OMe | H | L₅ | phenyl | H | —(CH₂)₂O— | —(CH₂)₂(OCH₂CH₂)₂— | —NHC(═O)NH— | CL |
| 64 | OMe | OMe | L₅ | phenyl | OEt | —(CH₂)₂O— | —(CH₂)₂(OCH₂CH₂)₂— | —NHC(═O)O— | GF2000 |
| 65 | OMe | OMe | L₅ | phenyl | F | —(CH₂)₂C(═O)NH— | —(CH₂)₂(OCH₂CH₂)₂— | —NHC(═O)NH— | UL-DADPA |
| 66 | OMe | OMe | L₅ | phenyl | F | —(CH₂)₂O— | —(CH₂)₂(OCH₂CH₂)₂— | —NHC(═O)O— | PS10 |
| 67 | OMe | OMe | L₅ | phenyl | F | —(CH₂)₂O— | —(CH₂)₂— | —NH— | AL |
| 68 | OMe | OMe | L₅ | phenyl | F | —(CH₂)₂O— | —(CH₂)₂(OCH₂CH₂)— | 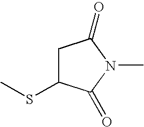 | UL-M |
| 69 | OMe | OMe | L₅ | pyridin | Cl | —(CH₂)₂O— | —(CH₂)₂(OCH₂CH₂)— | 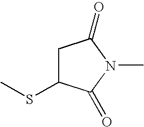 | UL-M |
| 70 | OMe | OMe | L₅ | phenyl | F | —(CH₂)₂O— | —(CH₂)₂(OCH₂CH₂)— | —C(═O)NH— | UL-DADPA |
| 71 | OMe | OMe | L₅ | pyridin | Cl | —(CH₂)₂O— | —(CH₂)₂(OCH₂CH₂)— | —SCH₂CH(OH)— | PS20 |
| 72 | OMe | OMe | L₅ | phenyl | F | —(CH₂)₂O— | —(CH₂)₂(OCH₂CH₂)— | —NH(C═O)— | CL |
| 73 | OMe | OMe | L₅ | phenyl | F | —(CH₂)₂O— | —(CH₂)₃C(═O)NH(CH₂)₃NH(CH₂)₄— | —(C═O)NH— | UL-DADPA |
| 74 | OMe | OMe | L₅ | phenyl | F | —(CH₂)₂O— | —(CH₂)₂NHC(═O)CH₂— | 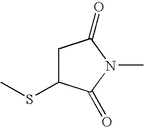 | UL-M |
| 75 | L₄ | OMe | H | phenyl | F | —O— | —(CH₂)₂(OCH₂CH₂)₂— | —NH— | -biotin- |
| | | | | | | | | | Neut-UL- |
| 76 | L₄ | OMe | H | phenyl | F | —O— | —(CH₂)₃NHC(═O)(CH₂)₂SS(CH₂)₂— | —NH— | -biotin- |
| | | | | | | | | | Neut-UL- |
| 77 | L₄ | OMe | H | phenyl | F | —O— | —(CH₂)₃NHC(═O)(CH₂)₄— | —NH— | -biotin- |
| | | | | | | | | | Av-UL |
| 78 | L₄ | OMe | H | phenyl | F | —O— | —(CH₂)₃NHC(═O)(CH₄)₄NHC(═O)(CH₂)₄— | —NH— | -biotin- |
| | | | | | | | | | Neut-UL- |
| 79 | L₄ | OMe | H | phenyl | F | —O— | —(CH₂)₃— | —NH— | -biotin- |
| | | | | | | | | | Strep-UL |
| 80 | L₄ | OMe | H | phenyl | F | —O— | —(CH₂)₂(OCH₂CH₂)₂— | —NH— | -biotin- |
| | | | | | | | | | Av-UL |
| 81 | L₄ | OMe | H | phenyl | F | —O— | —(CH₂)₂SCH₂C(═O)NH(CH₂)₆— | —NH— | -biotin- |
| | | | | | | | | | Av-UL |
| 82 | L₅ | OMe | H | phenyl | F | —O— | —(CH₂)₄SS(CH₂)₂C(═O)NH(CH₂)₆— | —NH— | -biotin- |
| | | | | | | | | | Av-UL |
| 82a | L₄ | OMe | H | phenyl | F | —O— | —(CH₂)₄SS(CH₂)₂C(═O)NH(CH₂)₆— | —NH— | -ImB- |
| | | | | | | | | | Av-UL |
| 83 | L₄ | OMe | H | pyridinyl | Cl | —O— | —(CH₂)₃NHC(═O)(CH₂)₄— | —NH— | -biotin- |
| | | | | | | | | | Av-UL |
| 84 | OEt | L₄ | H | phenyl | OEt | —O— | —(CH₂)₂(OCH₂CH₂)₂— | —NH— | -biotin- |
| | | | | | | | | | Av-UL |
| 85 | OEt | L₄ | H | phenyl | Br | —O— | —(CH₂)₂(OCH₂CH₂)₂— | —NH— | -biotin- |
| | | | | | | | | | Av-PS10- |
| 86 | OMe | L₄ | H | phenyl | F | —O— | —(CH₂)₄NHC(═O)(CH₂)₄— | —NH— | -biotin- |
| | | | | | | | | | Av-PS10 |
| 87 | OMe | L₄ | H | phenyl | F | —O— | —(CH₂)₃NHC(═O)(CH₂)₂SS(CH₂)₂— | —NH— | -biotin- |
| | | | | | | | | | Av-UL |
| 87 | OMe | L₄ | H | phenyl | F | —O— | —(CH₂)₃NHC(═O)(CH₂)₃SS(CH₂)₂— | —NH— | -Imb- |
| | | | | | | | | | Av-UL |
| 88 | OMe | L₄ | H | phenyl | F | —O— | —(CH₂)₃— | —NH— | -biotin- |
| | | | | | | | | | Av-UL |
| 88a | OMe | L₄ | H | phenyl | F | —O— | —(CH₂)₃— | —NH— | -Imb- |
| | | | | | | | | | Av-UL |
| 89 | OMe | L₄ | H | phenyl | F | —O— | (CH₂)₃NHC(═O)CH₂OCH₂C(═)NH(CH₂)₃(OCH₂CH₂)₃CH₂— | — | -biotin- |
| | | | | | | | | | Av-UL |
| 90 | OMe | OMe | H | phenyl | L₄ | —C(═O)NH— | —(CH₂)₃(OCH₂CH₂)₂(CH₂)₂— | —NH— | -biotin- |
| | | | | | | | | | Av-UL |
| 91 | OMe | OMe | H | phenyl | L₄ | —C(═O)NH— | —(CH₂)₂(OCH₂CH₂)₃— | —NH— | -biotin- |
| | | | | | | | | | Av-UL |
| 92 | OMe | OMe | H | phenyl | L₄ | —SS— | —(CH₂)₄NHC(═O)(CH)₂— | —NH— | -biotin- |
| | | | | | | | | | Av-UL |
| 93 | OMe | OMe | H | phenyl | L₄ | —O— | —(CH₂)₂(OCH₂CH₂)₂(CH₂)₂— | —NH— | -biotin- |
| | | | | | | | | | Av-UL |
| 95 | OMe | OMe | H | phenyl | L₄ | —S— | —(CH₂)C(═O)NH(CH₂)₂(OCH₂CH₂)₂— | —NH— | -biotin- |
| | | | | | | | | | Av-UL |
| 96 | OEt | —O(CH₂)₃OH | H | phenyl | L₄ | —O— | —(CH₂)₂(OCH₂CH₂)₂C(═O)NH(CH₂)₄— | —NH(C═O)— | CL |
| 97 | OEt | —O(CH₂)₃OH | H | phenyl | L₄ | —O— | —(CH₂)₂(OCH₂CH₂)₂— | 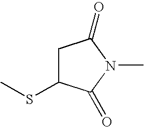 | UL-M |
| 98 | OMe | OMe | L₅ | phenyl | F | —(CH₂)₂O— | —(CH₂)₂— | —NH— | -biotin- |
| | | | | | | | | | Av-UL |
| 99 | OMe | OMe | L₅ | pyridin | Cl | —(CH₂)₂O— | —(CH₂)₂— | —NH— | -biotin- |
| | | | | | | | | | -Av-UL |
| 100 | OMe | OMe | L₅ | phenyl | F | —(CH₂)₂O— | —(CH₂)₂(OCH₂CH₂)— | 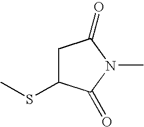 | UL-M |
| 101 | OMe | OMe | L₅ | phenyl | F | —(CH₂)₂O— | —(CH₂)₂(OCH₂CH₂)— | —NH— | -biotin- |
| | | | | | | | | | Av-UL |
| 102 | OMe | OMe | L₅ | phenyl | F | —(CH₂)₂O— | —(CH₂)₂(OCH₂CH₂)NHC(═O)(CH₂)₄— | -biotin- | |
| | | | | | | | | | Av-UL |
| 103 | OEt | —O(CH₂)₃OH | L₅ | phenyl | F | —(CH₂)₂O— | —(CH₂)₂(OCH₂CH₂)NHC(═O)(CH₂)₄— | —NH— | -biotin- |
| | | | | | | | | | Av-UL |
| 103a | OEt | —O(CH₂)₃OH | L₅ | phenyl | F | —(CH₂)₂O— | —(CH₂)₂(OCH₂CH₂)NHC(═O)(CH₂)₄— | —NH— | -Imb- |
| | | | | | | | | | Av-UL |
| 104 | OEt | OH | L₅ | phenyl | OEt | —(CH₂)₂O— | —(CH₂)₂(OCH₂CH₂)NHC(═O)(CH₂)₄— | —NH— | -biotin- |
| | | | | | | | | | Av-UL |
| 105 | H | —O(CH₂)₃OH | L₅ | phenyl | OEt | —(CH₂)₂O— | —(CH₂)₂(OCH₂CH₂)NHC(═O)(CH₂)₄— | —NH— | -biotin- |
| | | | | | | | | | Av-UL |
| 106 | OMe | OMe | L₅ | phenyl | F | —(CH₂)₂O— | —(CH₂)₂(OCH₂CH₂)— | —NH— | -biotin- |
| | | | | | | | | | Av-UL |
| 107 | OMe | OMe | L₅ | phenyl | F | —(CH₂)₂O— | —(CH₂)₂— | —NH— | -biotin- |
| | | | | | | | | | Av-UL |
| 108 | OMe | OMe | L₅ | phenyl | F | —(CH₂)₂O— | —(CH₂)₂(OCH₂CH₂)SS(CH₂)₂— | —NH— | -biotin- |
| | | | | | | | | | Av-UL |
| 109 | OMe | OMe | L₅ | phenyl | OMe | —(CH₂)₂O— | —(CH₂)₂(OCH₂CH₂)— | —NH— | -biotin- |
| | | | | | | | | | Strep-UL |
| 110 | OMe | H | L₅ | phenyl | OMe | —(CH₂)₂O— | —(CH₂)₂(OCH₂CH₂)— | —NH— | -biotin- |
| | | | | | | | | | Strep-UL |
| 111 | L₄ | OMe | H | CH₂Ph | 2-Cl, 6-Me | —O— | —(CH₂)₂(OCH₂CH₂)₃— | —NHC(═O)O— | PS10 |
| 112 | L₄ | OMe | H | Indanyl | H | —O— | —(CH₂)₂(OCH₂CH₂)₃— | —NHC(═O)O— | GF2000 |
| 113 | L₄ | OMe | H | CH₂Ph | H | —O— | —(CH₂)₂OCH₂CH₂)₂— | 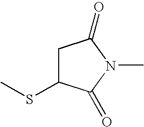 | UL-M |
| 114 | L₄ | OMe | H | CH₂Ph | 2-Cl, 6-Me | —O— | —(CH₂)₃NHC(═)CH₂SS(CH₂)₄— | —NHC(═O)O— | GF2000 |
| 115 | OMe | L₄ | H | indanyl | H | —O— | —(CH₂)(OCH₂CH₂)₂— | —C(═O)O— | SL |
| 116 | OMe | OMe | L₅ | tetralinyl | H | —(CH₂)₂O— | —(CH₂)₂(OCH₂CH₂)₂— | —NHC(═O)O— | GF2000 |
| 117 | OMe | OMe | H | CH₂Ph | 2-Cl, 5-L₄ | —C(═O)NH— | —(CH₂)₃(OCH₂CH₂)₂(CH₂)₂— | —NHC(═O)O— | GF2000 |
| 118 | OMe | OMe | H | Indanyl | L₄ | —O— | —(CH₂)₂(OCH₂CH₂)₂(CH₂)₂— | —NH— | -biotin- |
| | | | | | | | | | Strep-UL |
| 118a | OMe | OMe | H | Indanyl | L₄ | —O— | —(CH₂)₂(OCH₂CH₂)₂(CH₂)₂— | —NH— | -Imb- |
| | | | | | | | | | Strep-UL |
| 119 | L₄ | OMe | H | Indanyl | L₄ | —O— | —(CH₂)₃NHC(═O)(CH₂)₂SS(CH₂)₂— | —NH— | -biotin- |
| | | | | | | | | | Strep-UL |
| 120 | L₄ | OMe | H | —CH₂Ph | 2,6-di-Cl | —O— | —(CH₂)₃NHC(═O)(CH₂)₄— | —NH— | -biotin- |
| | | | | | | | | | Strep-UL |
| 121 | OMe | L₄ | H | —CH₂Ph | Cl | —O— | —(CH₂)₃— | —NH— | -biotin- |
| | | | | | | | | | Strep-UL |
| 122 | OEt | —O(CH₂)₃OH | L₅ | —CH₂Ph | 2,6-di-Cl | —(CH₂)₂O— | —(CH₂)₂(OCH₂CH₂)NHC(═O)(CH₂)₄— | —NH— | -biotin- |
| | | | | | | | | | Strep-UL |
| 123 | OMe | OMe | H | phenyl | 3-L₄ | —C(═O)NH— | —(CH₂)₂(OCH₂CH₂)₃— | —NH— | -biotin- |
| | | | | | | | | | Strep- |
| | | | | | | | | | PS20 |
| 124 | L₄ | OMe | H | phenyl | 3-F | —O— | —(CH₂)₂(OCH₂CH₂)₂NHC(═)(CH₂)₂C(═O) | —NH— | -biotin- |
| | | | | | | | NH(CH₂)₃(OCH₂CH₂)₃CH₂— | | Av-PS20 |

1.D Methods of Synthesis

The compounds of the present invention may be prepared by any number of processes as described generally below and more specifically as described in the Examples which follow herein.

During any of the processes for preparation of the compounds of the present invention described herein, it may be necessary and/or desirable to protect sensitive or reactive groups on any of the molecules concerned (for example hydroxy, amino, thio, oxo or carboxy groups). This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

### Method A

Compounds of Formula (I) may be prepared according to the process outlined in Method A.

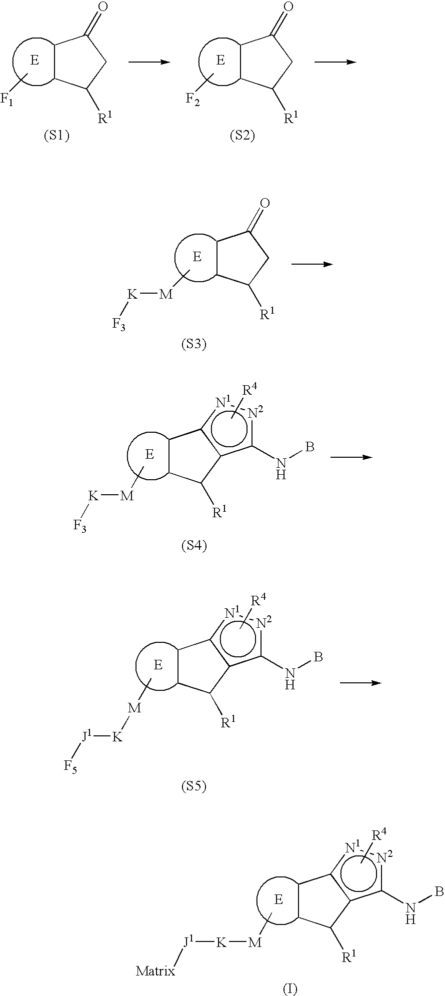

Accordingly, a compound of Formula (S1), wherein F₁ is prepared as outlined in U.S. patent application Ser. No. 10/438,152, “filed May 14, 2003, and PCT/US/03/15193, filed May 13, 2003, may be converted to a compound of Formula (S2) via methods known to those skilled in the art such as de-methylation, nitro-reduction, oxidation, reaction with an isocyanate, hydrolysis of a cyanide substituent, and carboxylate. Reduction of a nitro compound (F₁=NO₂) can be achieved by hydrogenation in the presence of a catalyst such as Pd—C or Raney-Nickel, or by a tin chloride reduction, to give an amine (F₂═NH₂). In another example, de-methylation of a methoxy-substituted aryl compound (F₁=OCH₃) with BBr₃ in methylene chloride or by heating with KCN/DMSO affords a phenolic compound (F₂=OH). Reduction of a disulfide (F₂=S₂) compound with sodium borohydride or the like affords a sulhydryl compound (F₂=SH).

A compound of Formula (S2) may be reacted with an appropriate linker reagent to prepare a compound of Formula (S3) wherein M is selected from —NHC(═O)—, NH(C═O)NH—, —NH(C═O)O—, —C(═O)NH, —O(C═O)NH—, —OC(═O)O—, —O(C═O)—, —(C═O)O—, —O—, —NH—, —(CH₂)₁₋₃O—, or —S—. The reactions can be achieved as shown in the following schemes. Some reactions in which compounds contain sensitive functional groups may require general protections and deprotections known to those skilled in the art.

Compounds of Formula (S2) wherein F₂ is hydroxyl may be converted according to known methods to form intermediate compounds (S3a), and F₃ is an appropriate functional group, protected or latent groups for the later transformations. Alternatively, Compounds of Formula (S2) wherein F₂ group is an amino group may be transformed to compounds of Formula (S3b-e) via known coupling chemistry with various acids, reductive amination of aldehydes, reaction with isocynates, and acylation with carbamyl chlorides. Similarly, compounds of Formula (S2) wherein F₂ is a sulhydryl group can be transformed to compounds of Formula (S3f-g) via alkylation in an aprotic solvent with a base such as sodium carbonate, potassium carbonate, cesium carbonate, sodium hydride, or sodium hydroxide, reacting with electrophiles or Michael reaction to maleimide. Additionally, compounds of Formula (S2) wherein F₂ is a carboxylic acid can be converted to compounds of Formula (S3h) through coupling reactions with various amines.

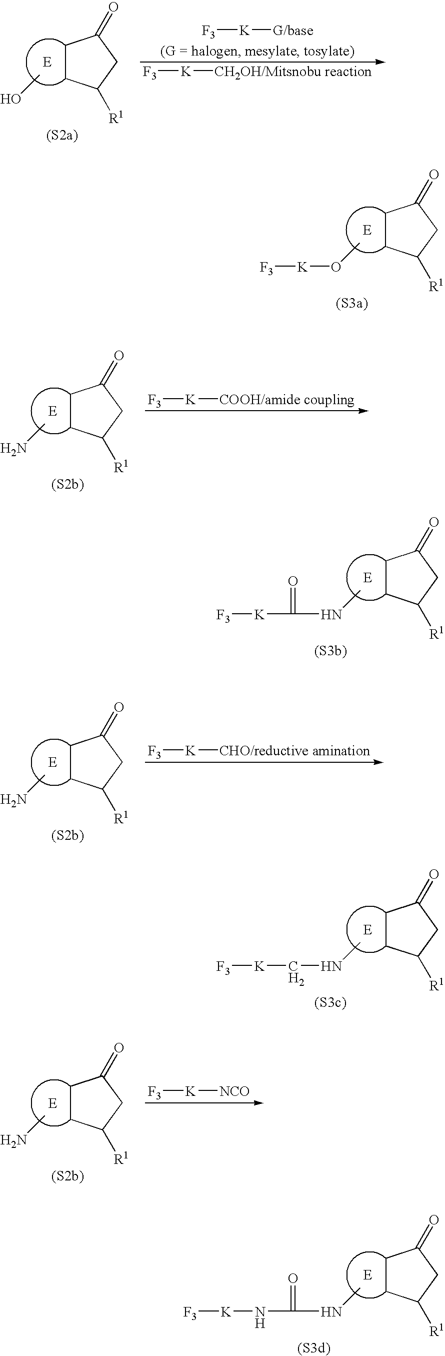

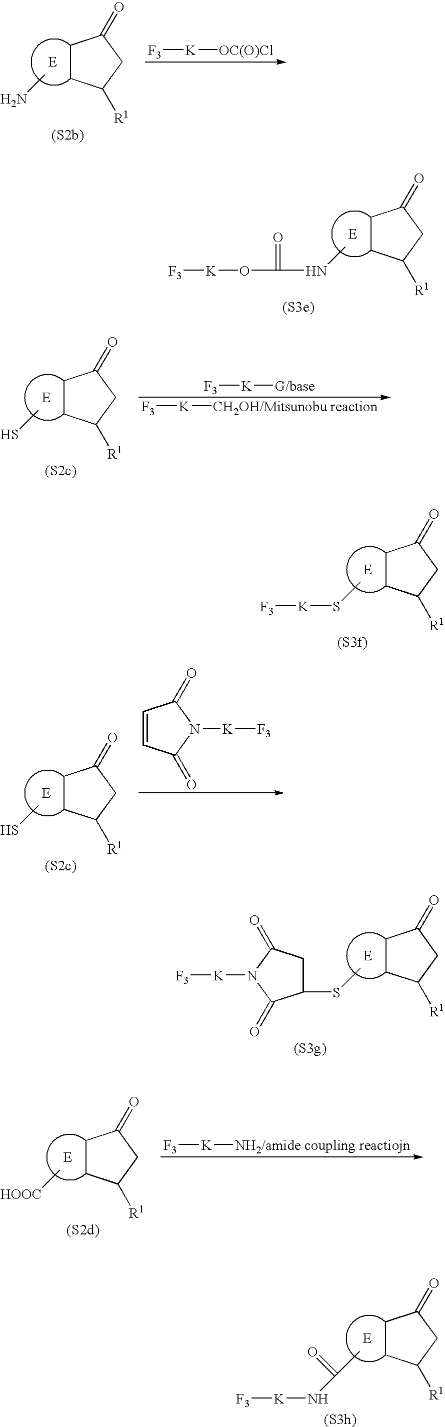

One skilled in the art will recognize that the above examples for the incorporation of desired linker functional groups into the compounds of Formula (S3) are not intended to be all inclusive, but rather are intended to provide examples of known chemistry of Formula (S3) by the known methods. The conversions of compounds of Formula (S3) to compounds of Formula (S₄) have been described in detail in U.S. patent application Ser. No. 10/438,152, “filed May 14, 2003, and PCT/US/03/15193, filed May 13, 2003. Compounds of Formula (S4) wherein F₃ are protected or unprotected functional groups are transformed to compounds containing F₅ wherein F₅ are hydroxyl, amino, sulhydryl, isocynate, carbonyldiimidazole (CDI), azide and halogen groups can be further coupled to selected matrixes (or biotin). Examples of reactions are shown in the following schemes.

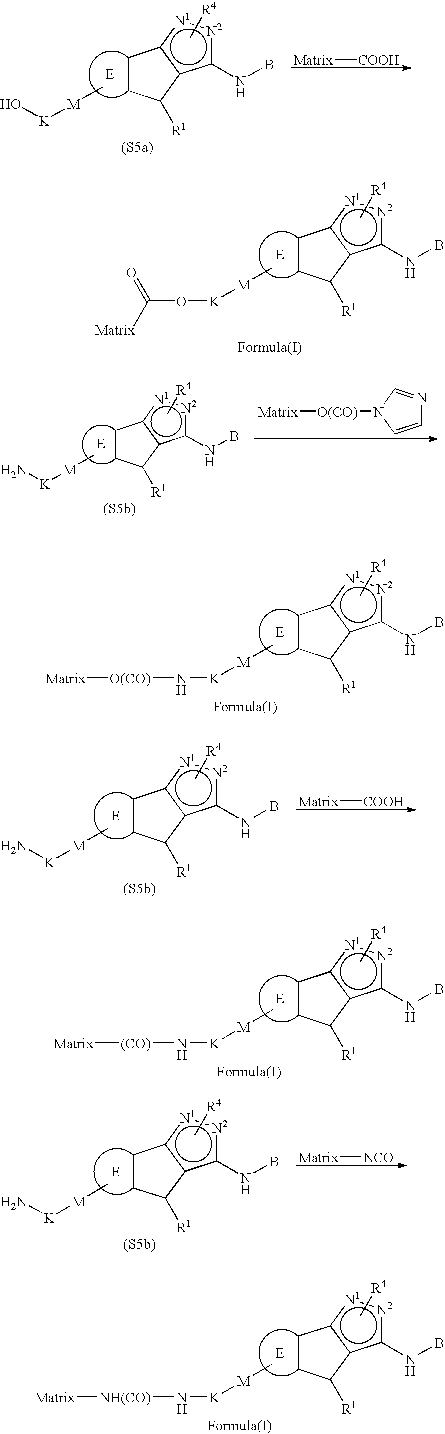

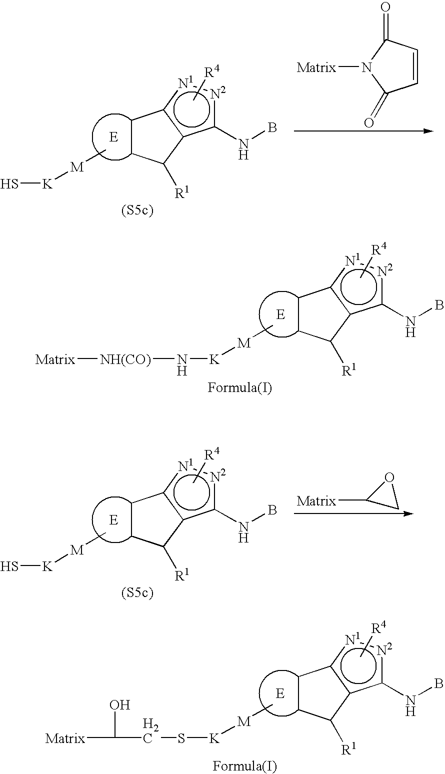

One skilled in the art will recognize that the above examples for the inclusion of desired linker functional groups into the compounds of Formula (I) are not intended to be all inclusive, but rather are intended to provide examples of known chemistry of Formula (I) by the known methods.

### Method B

Compounds of Formula (I) may be prepared according to the process outlined in Method B.

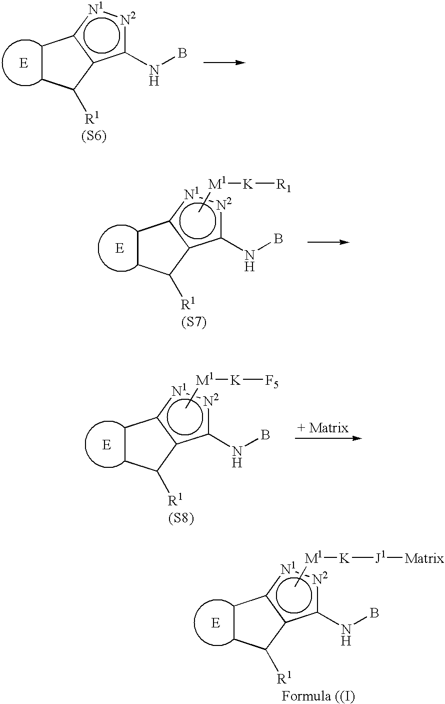

Accordingly, a compound of Formula (S6), prepared described in U.S. patent application Ser. No. 10/438,152, “filed May 14, 2003, and PCT/US/03/15193, filed May 13, 2003, was converted to a compound of Formula (S7) via known procedures such as alkylation with an appropriate electrophile, urea formation by reaction with isocyanate, or carbamate formation through reaction with carbamyl chloride. This transformation is exemplified in the following schemes. Reactions of compounds of Formula (S6) in general give two regioisomers which can be separated by silica gel column chromatography or reverse phase column chromatography.

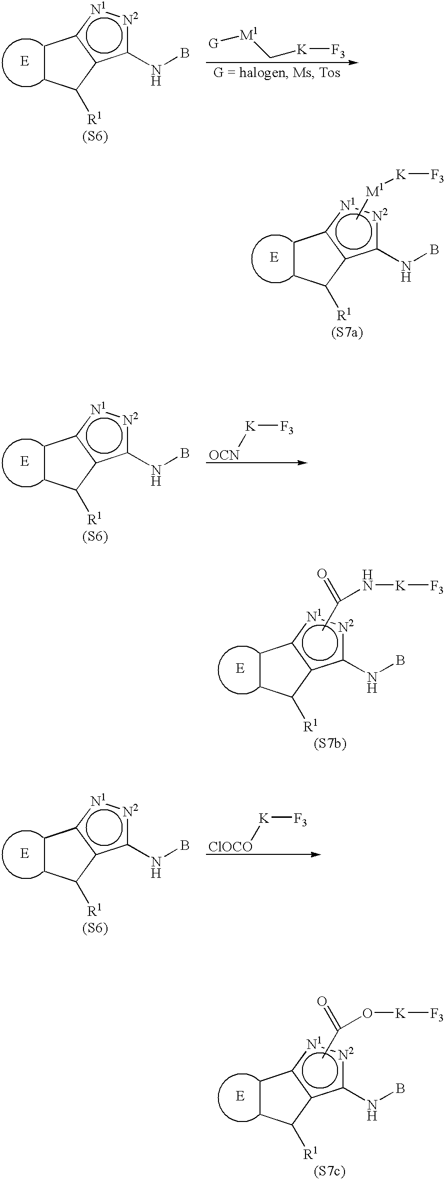

One skilled in the art will recognize that the above examples for the inclusion of desired linker functional groups into the compounds of Formula (S6) are not intended to be all inclusive, but rather are intended to provide examples of known chemistry of Formula (S7) by the known methods. 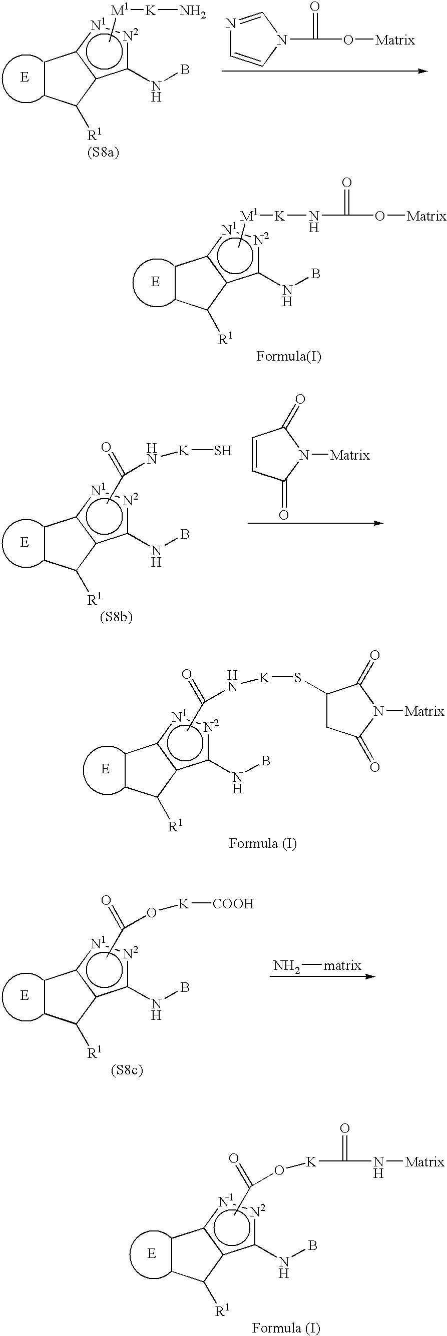
Accordingly, a compound of Formula (S8) was converted to a compound of Formula (I) via the known methods as exemplified. One skilled in the art will recognize that the above examples for the inclusion of desired linker functional groups (J) into the compounds of Formula (I) are not intended to be all inclusive, but rather are intended to provide examples of known chemistry of Formula (I) by the known methods.

### Method C

Compounds of Formula (I) may be prepared according to the process outlined in Method C.

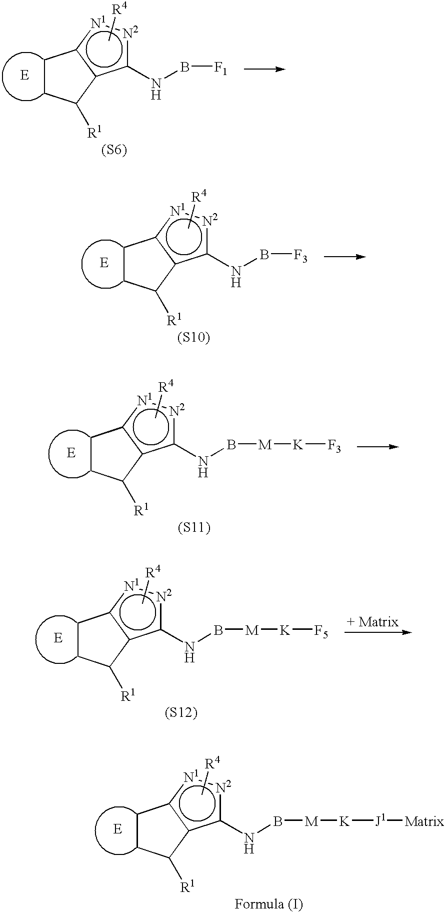

Accordingly, a compound of Formula (S6) wherein F₁ is selected from nitro, amino, cyano, alkylcarboxylate, aldehyde, hydroxymethyl, halogen, or hydroxy was transferred to a compound of Formula (S10) wherein F3 is selected from amino, hydroxyl, carboxyaldehyde, isocyanate, carboxylic acid, hydroxy, or sulhydryl via the known methods as described, such as de-methylation, and reduction of a nitro group.

### Method D

Compounds of Formula (I) may be prepared according to the process outlined in Method D.

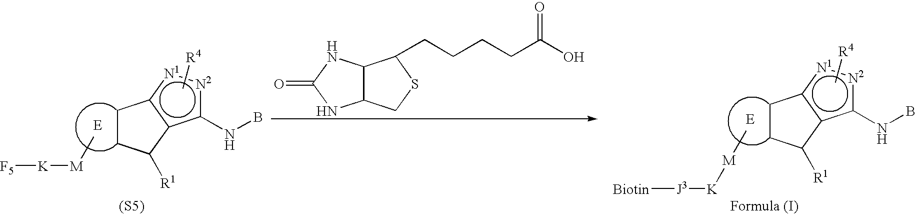

Accordingly, a compound of Formula (S5) was coupled to biotin to give a compound of Formula (I). Examples of preparations are shown in the following schemes.

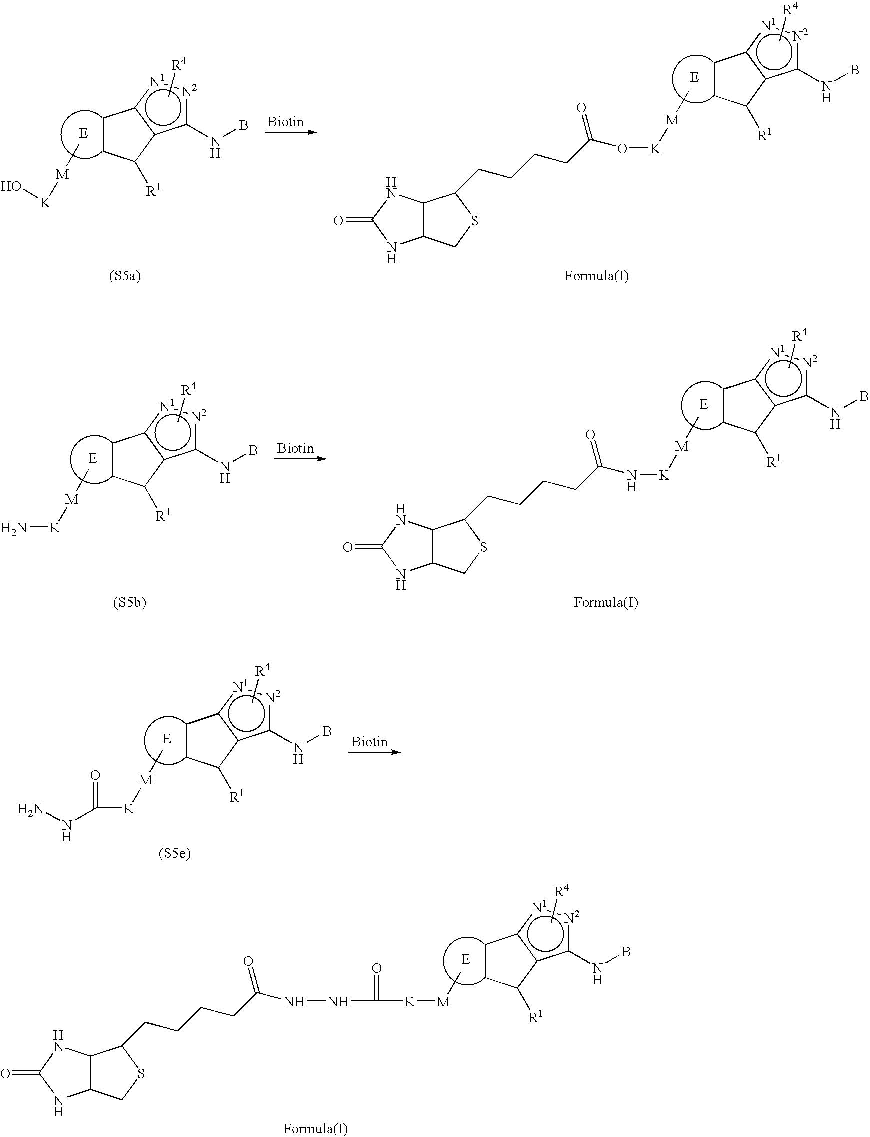

### Method E

Compounds of Formula (I) may be prepared according to the process outlined in Method E.

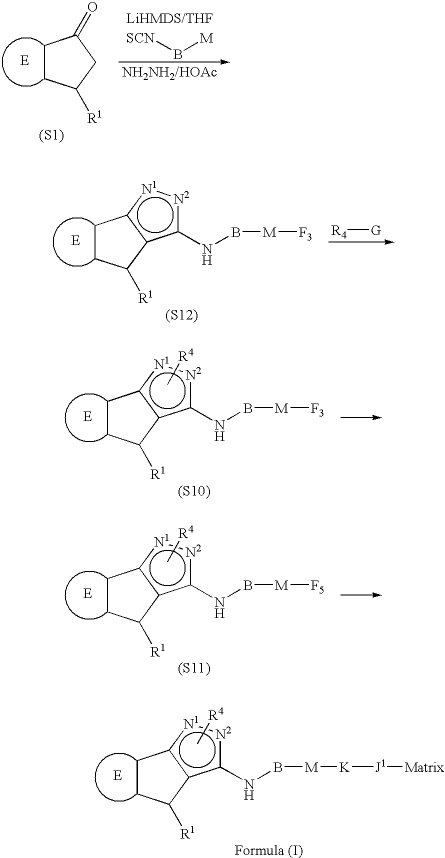

General chemistry for coupling the gels or supports with the biomolecules are shown in the following reaction schemes:

ReactiGel® coupling chemistry:

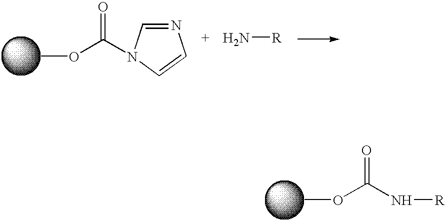

AminoLink® coupling chemistry:

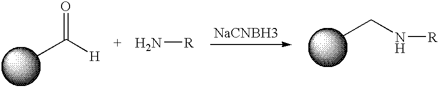

SulfoLink® coupling chemistry:

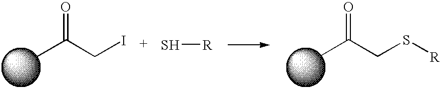

CarboLink™ coupling chemistry:

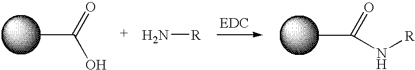

UltraLink™ DADPA coupling chemistry:

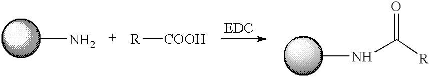

CarboLink™ hydrazide coupling chemistry:

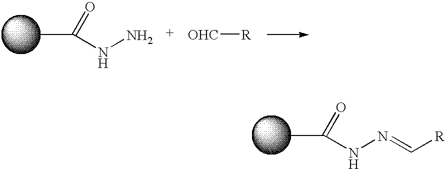

UltraLink™ maleamide coupling chemistry, which is synthesized as shown below:

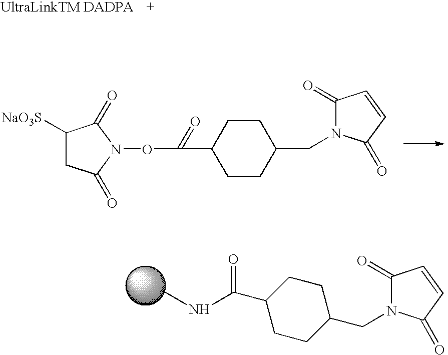

### E. EXAMPLES

Representative compounds of the present invention synthesized by the aforementioned methods are presented below. Examples of the synthesis of specific compounds are presented thereafter.

In the table below, 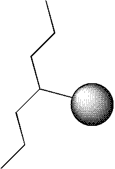
is used to represent avidin or streptavidin bound to matrix, wherein 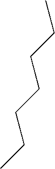
represents the protein avidin or streptavidin, and

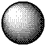

represents a matrix as defined herein.

| Cpd | |
|---|---|
| 1 | 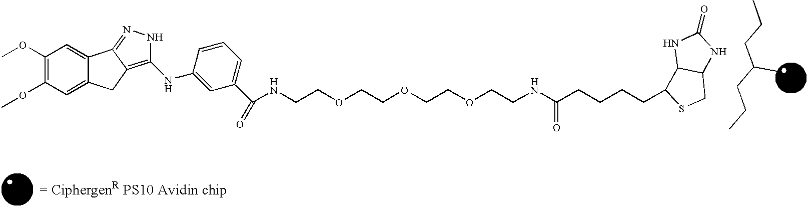 |
| 2 | 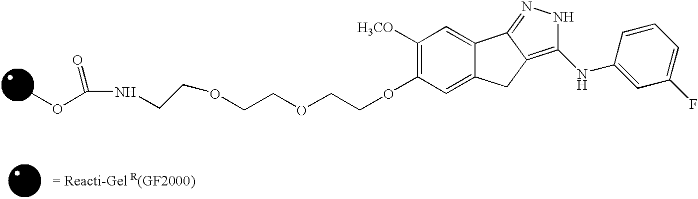 |
| 3 | 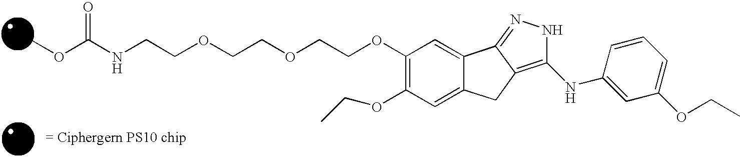 |
| 4 | 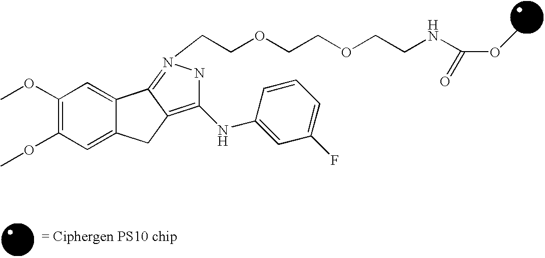 |
| 5 | 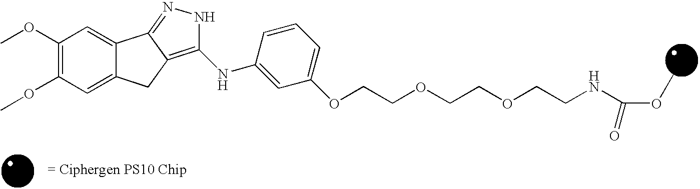 |
| 6 | 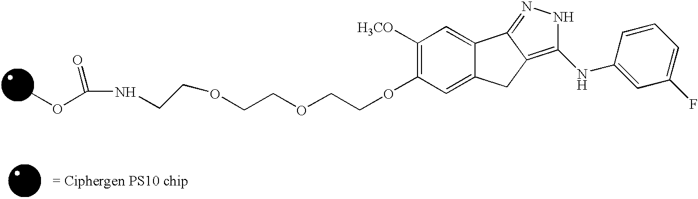 |
| 7 | 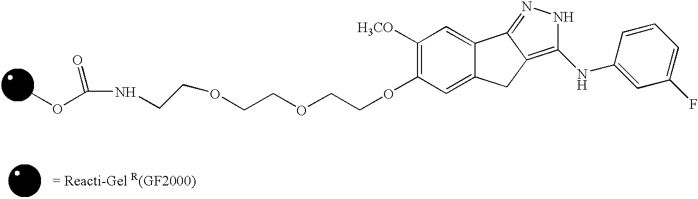 |
| 8 | 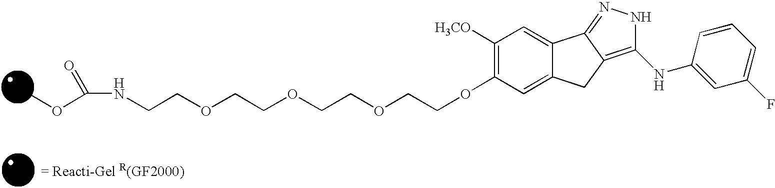 |
| 9 | 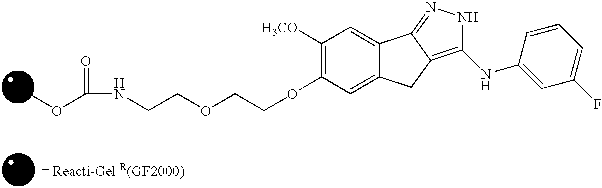 |
| 10 | 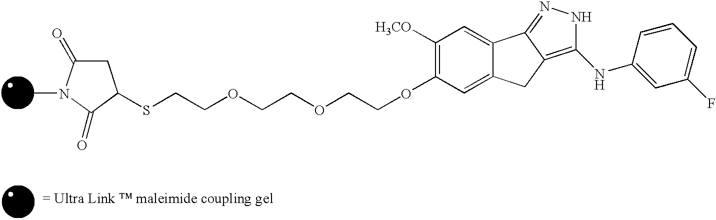 |
| 11 | 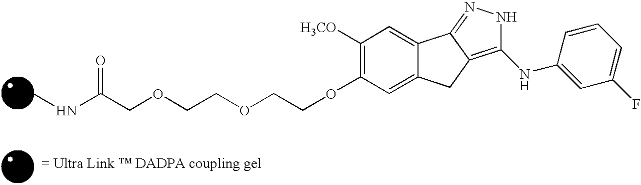 |
| 11a | 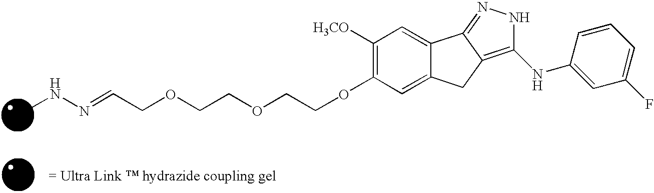 |
| 12 | 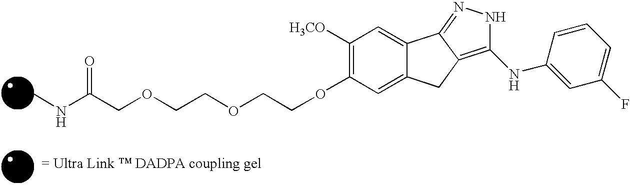 |
| 13 | 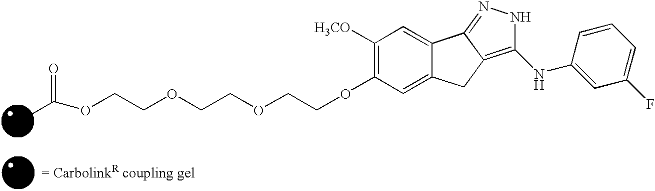 |
| 14 | 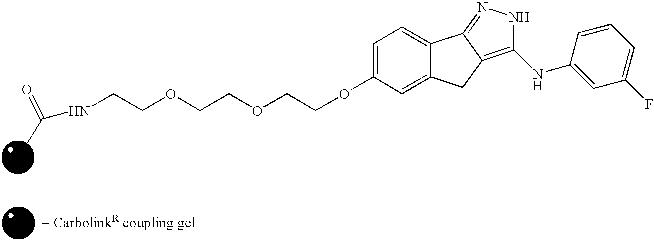 |
| 15 | 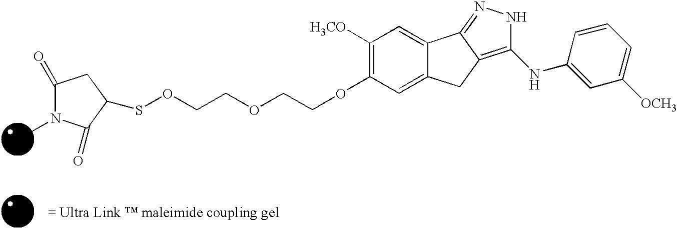 |
| 16 | 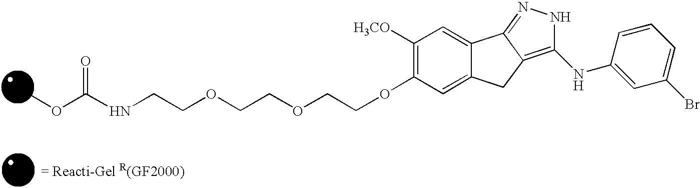 |
| 17 | 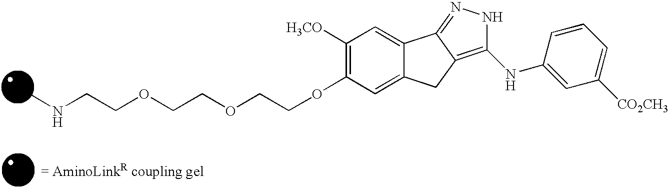 |
| 18 | 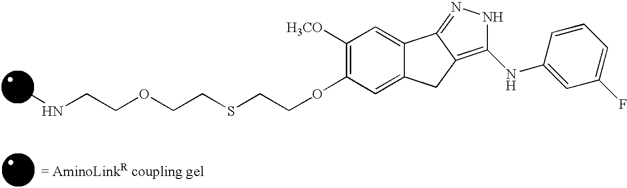 |
| 19 | 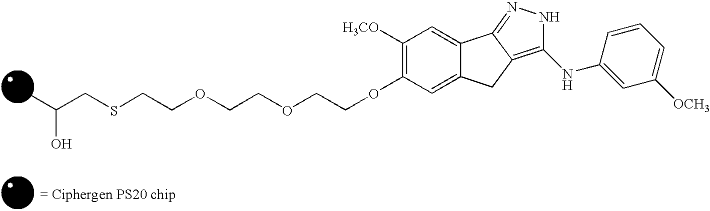 |
| 20 | 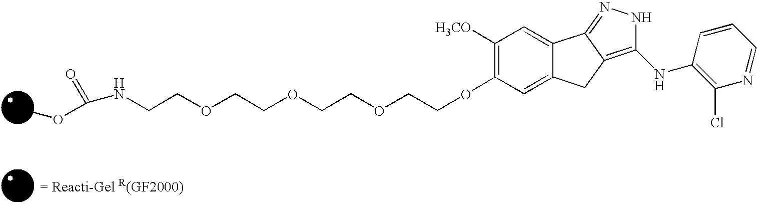 |
| 21 | 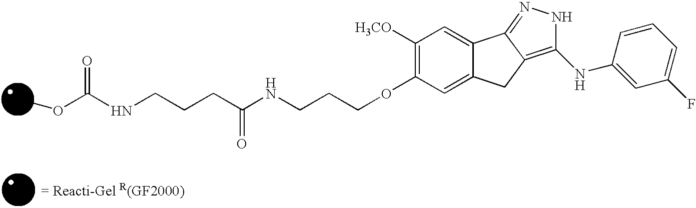 |
| 22 | 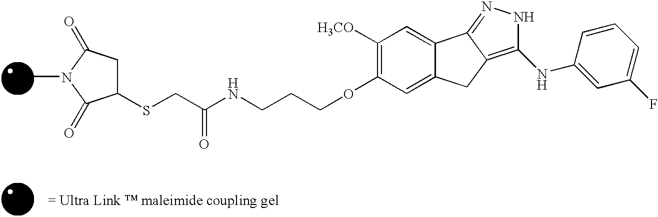 |
| 23 | 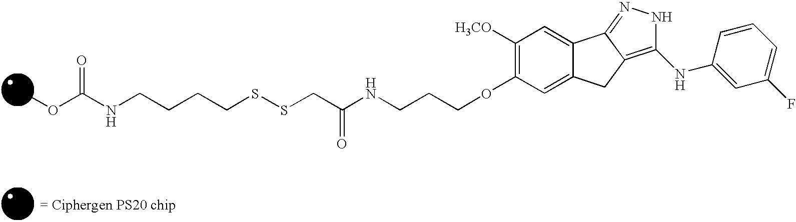 |
| 24 | 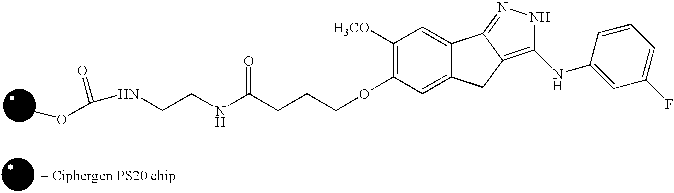 |
| 25 | 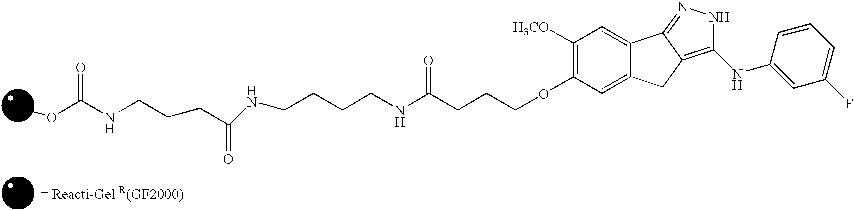 |
| 26 | 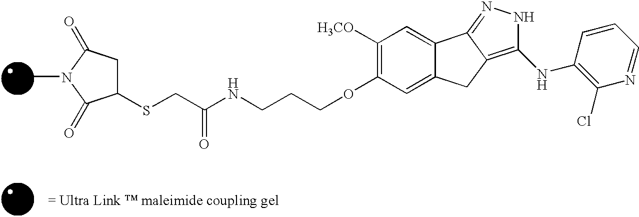 |
| 27 | 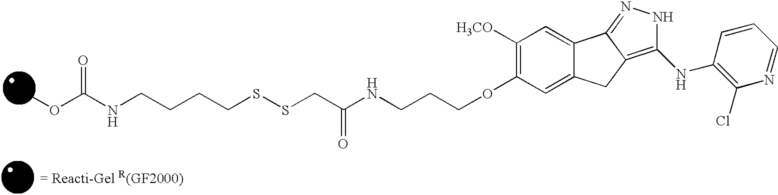 |
| 28 | 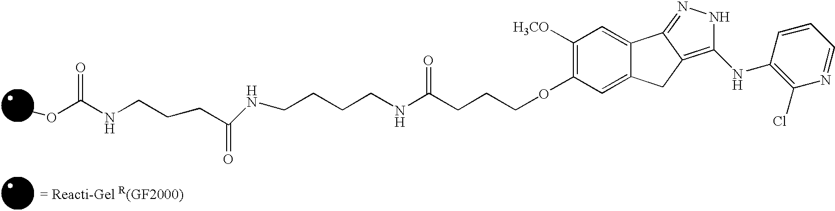 |
| 29 | 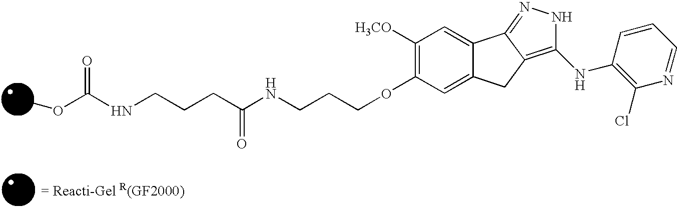 |
| 30 | 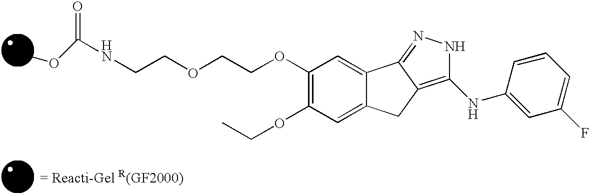 |
| 31 | 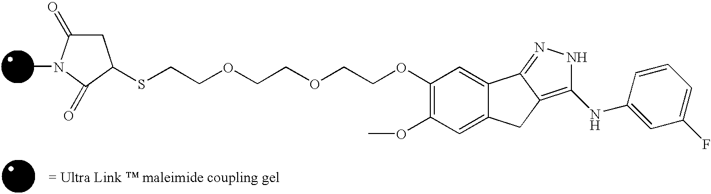 |
| 32 | 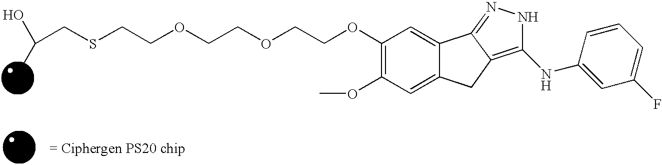 |
| 33 | 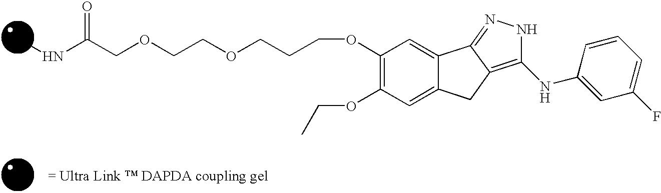 |
| 34 | 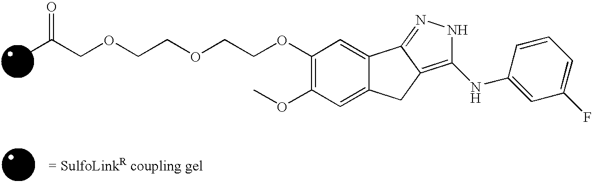 |
| 35 | 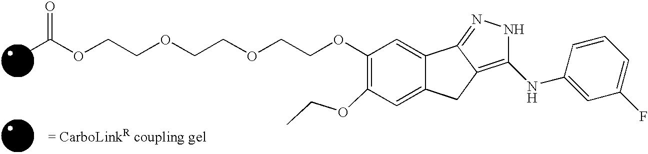 |
| 36 | 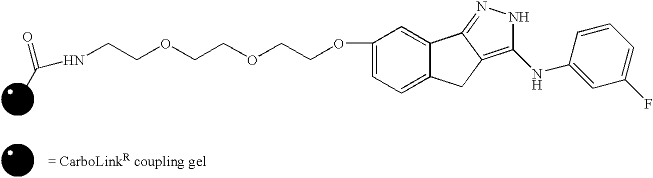 |
| 37 | 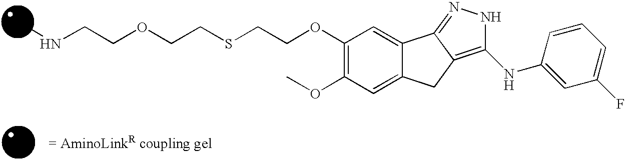 |
| 38 | 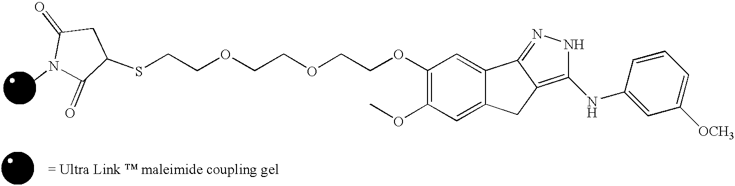 |
| 39 | 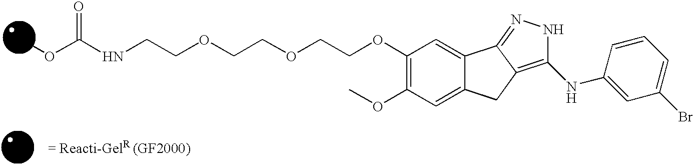 |
| 40 | 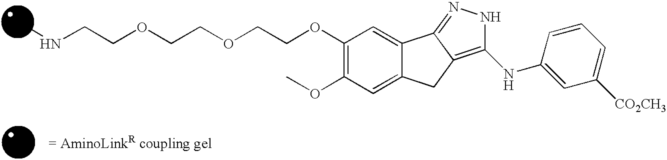 |
| 41 | 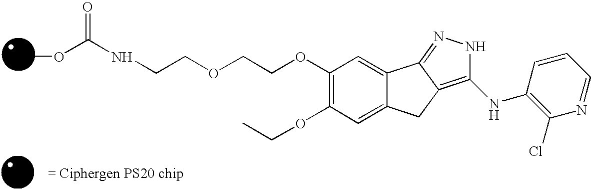 |
| 42 | 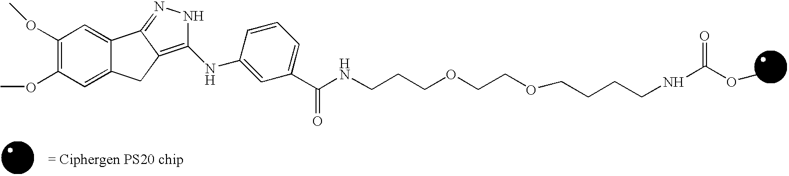 |
| 43 | 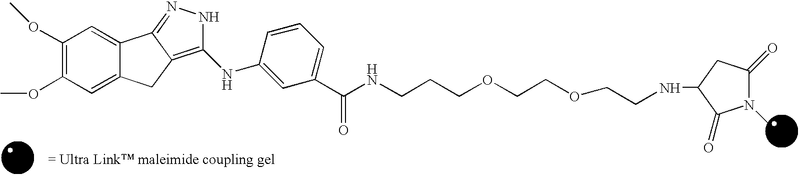 |
| 44 | 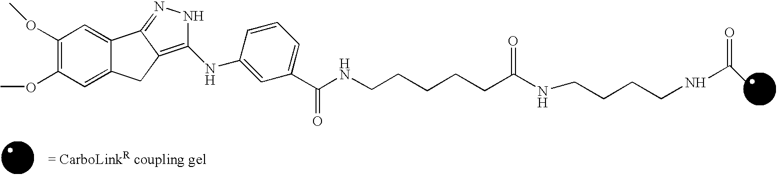 |
| 45 | 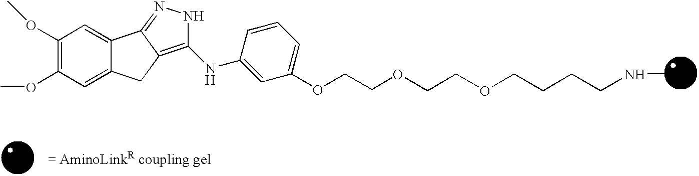 |
| 46 | 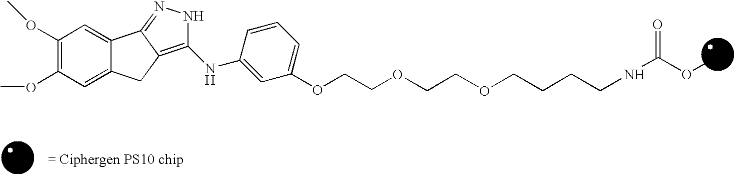 |
| 47 | 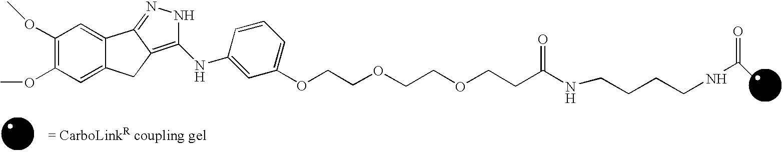 |
| 48 | 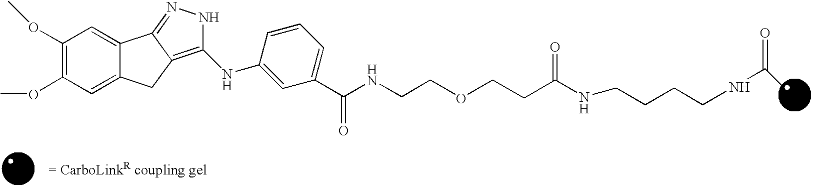 |
| 49 | 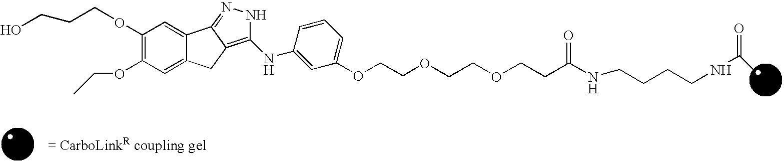 |
| 50 | 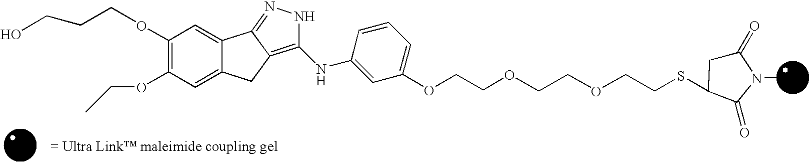 |
| 51 | 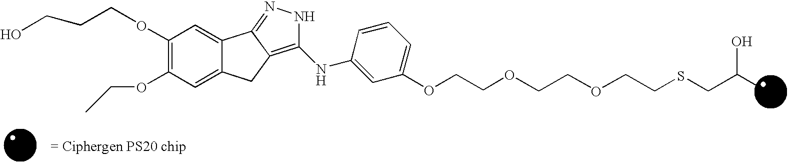 |
| 52 | 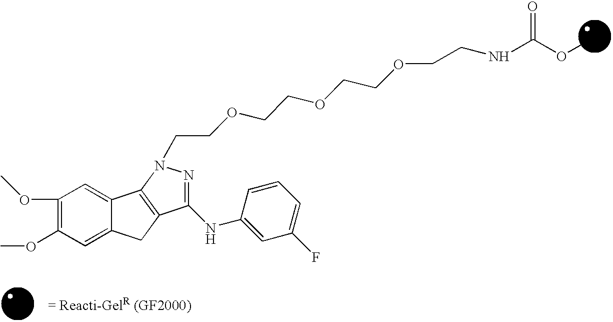 |
| 53 | 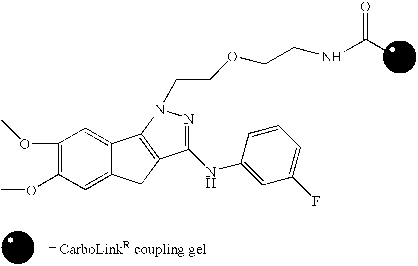 |
| 54 | 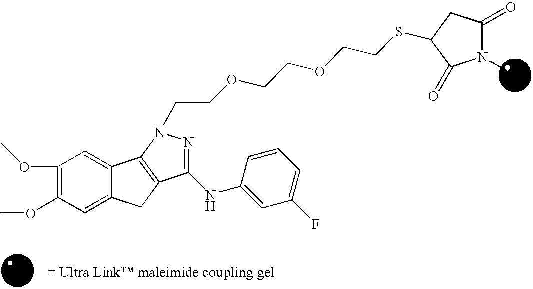 |
| 55 | 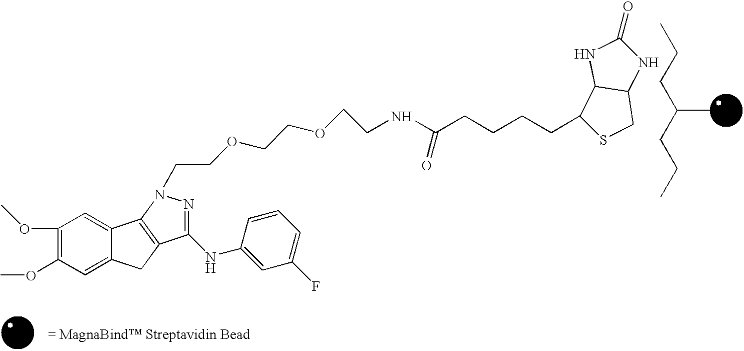 |
| 56 | 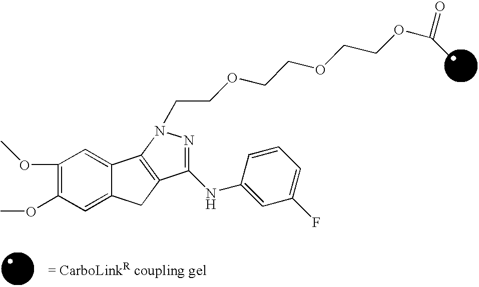 |
| 57 | 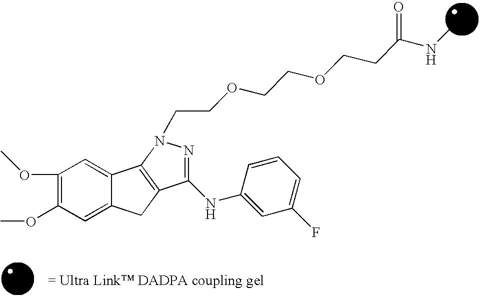 |
| 58 | 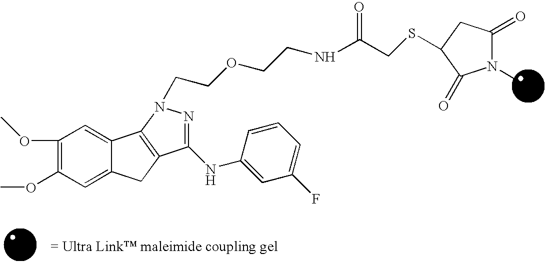 |
| 59 | 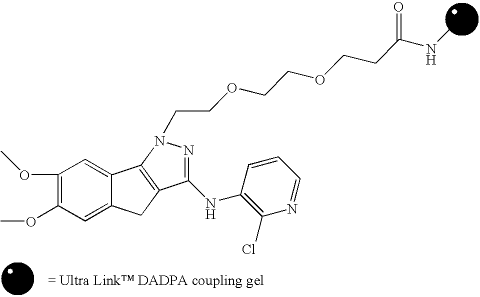 |
| 60 | 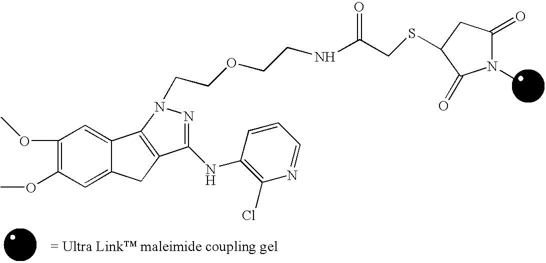 |
| 61 | 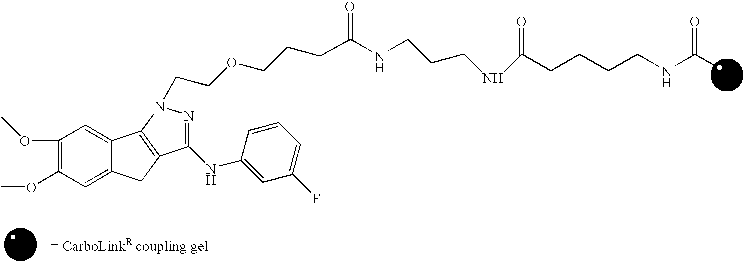 |
| 62 | 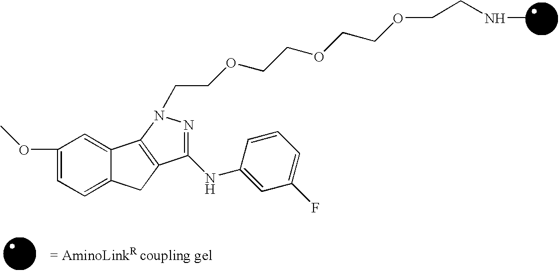 |
| 63 | 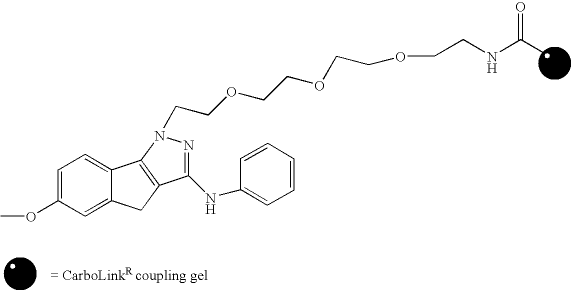 |
| 64 | 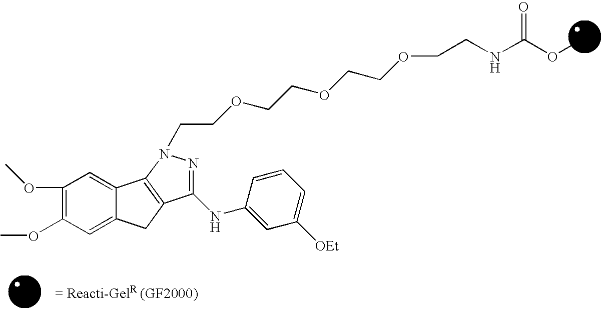 |
| 65 | 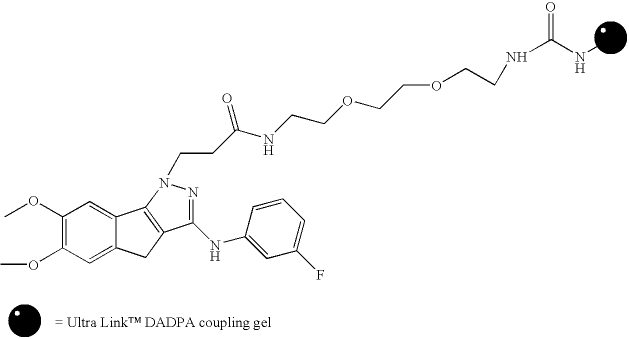 |
| 66 | 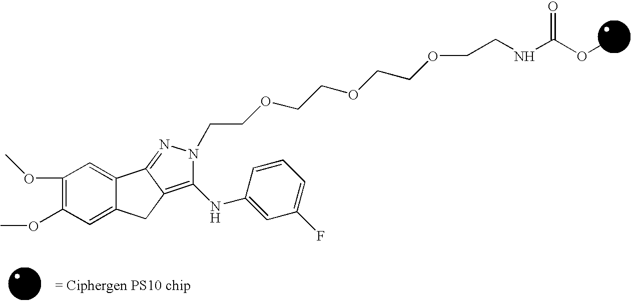 |
| 67 | 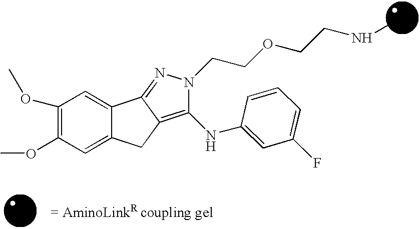 |
| 68 | 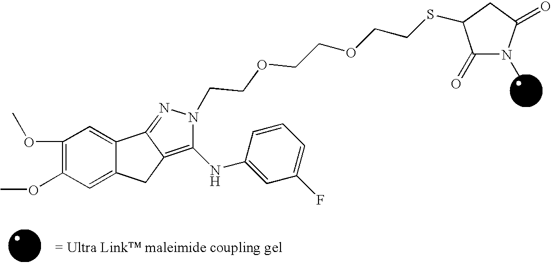 |
| 69 | 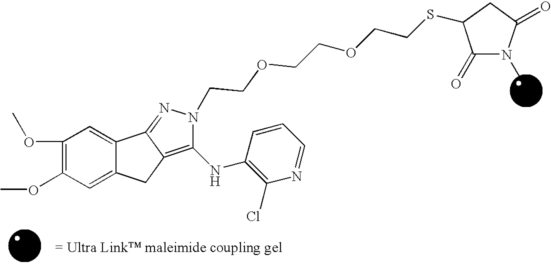 |
| 70 | 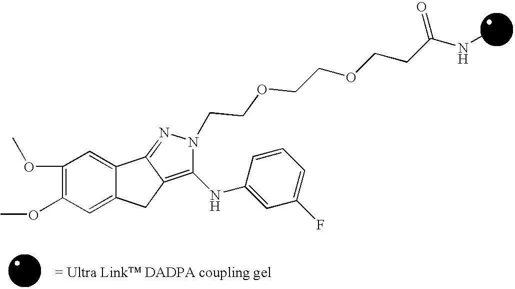 |
| 71 | 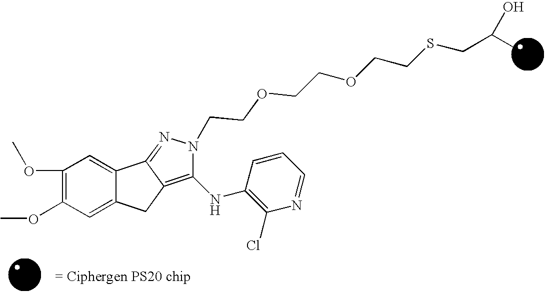 |
| 72 | 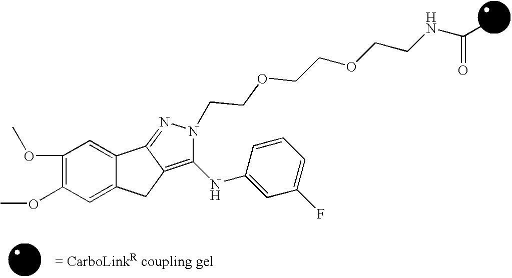 |
| 73 | 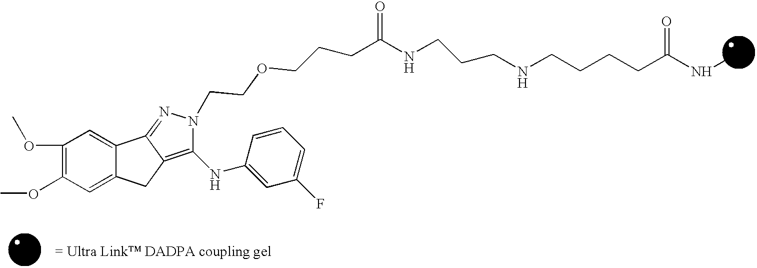 |
| 74 | 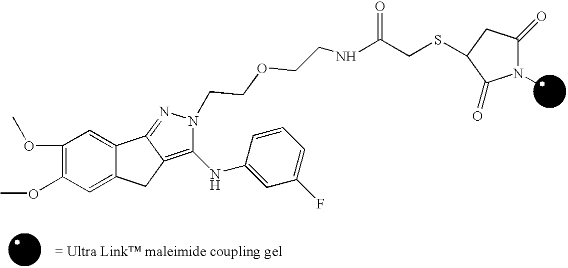 |
| 75 | 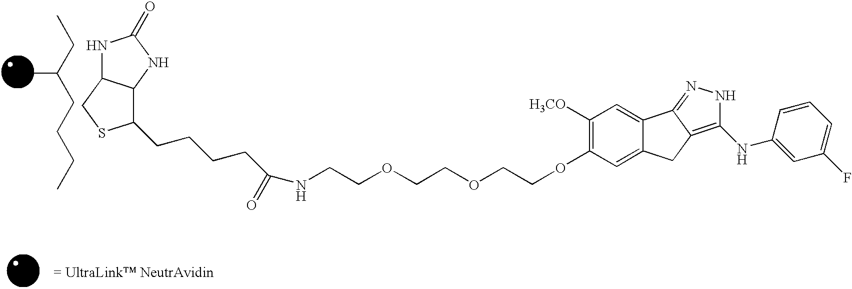 |
| 76 | 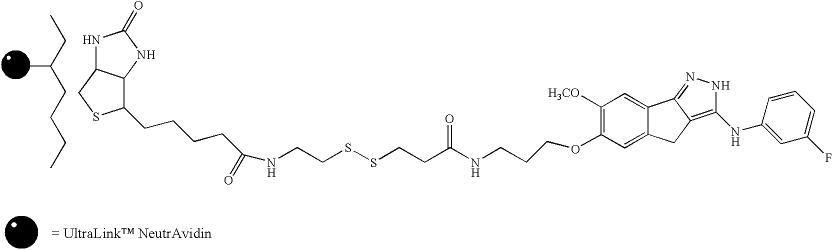 |
| 77 | 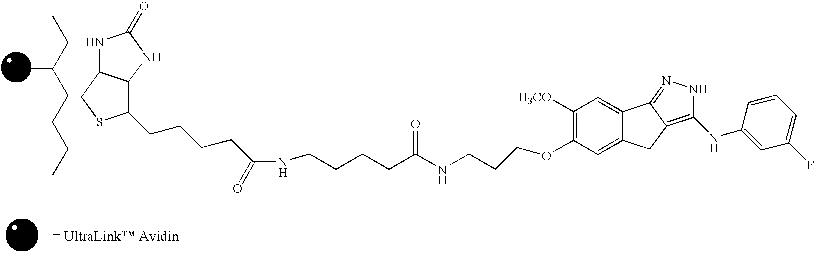 |
| 78 | 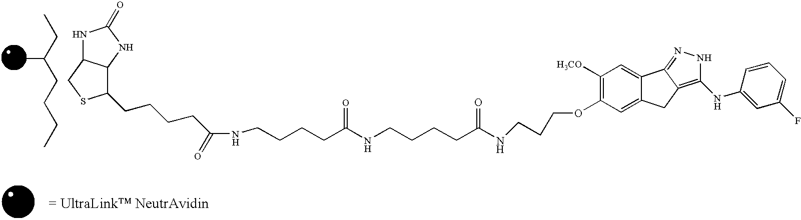 |
| 79 | 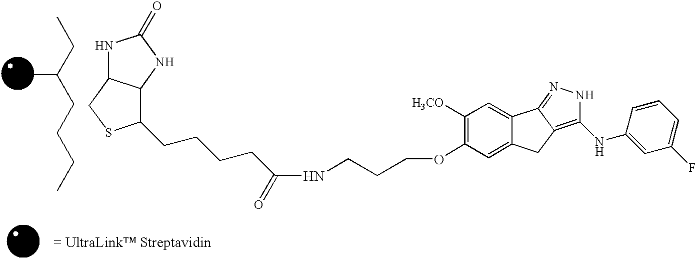 |
| 80 | 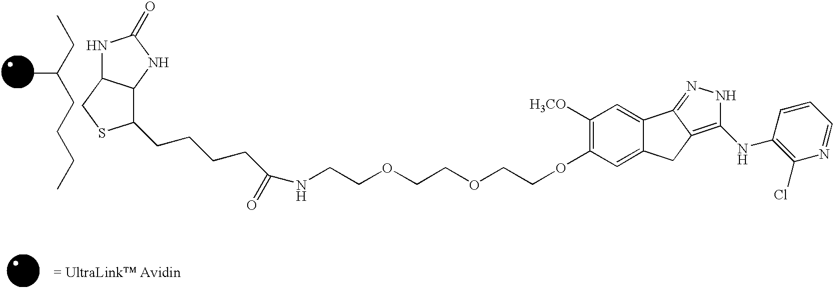 |
| 81 | 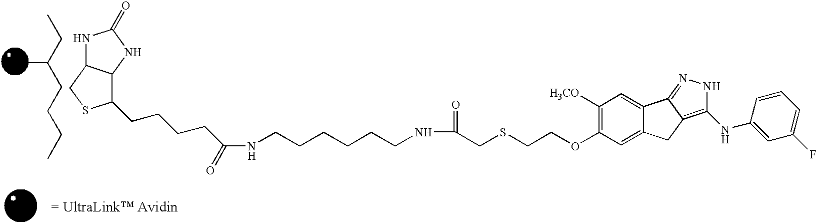 |
| 82 | 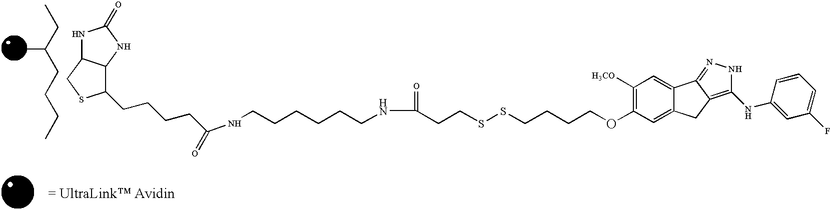 |
| 82a | 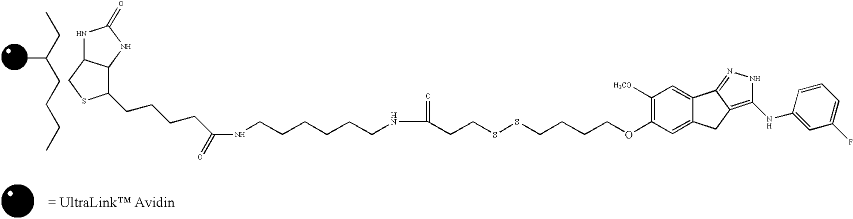 |
| 83 | 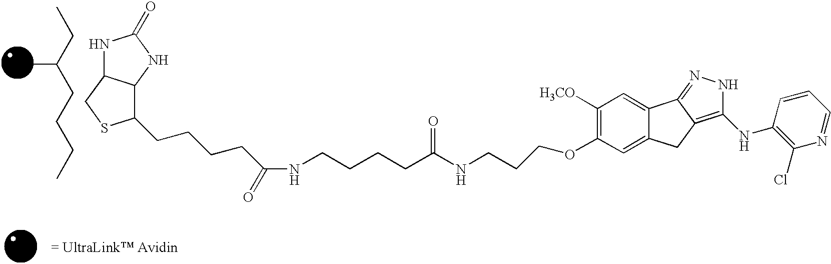 |
| 84 | 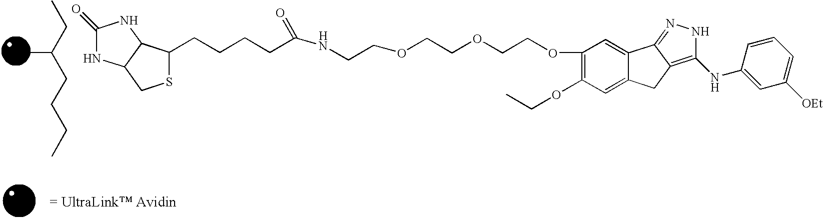 |
| 85 | 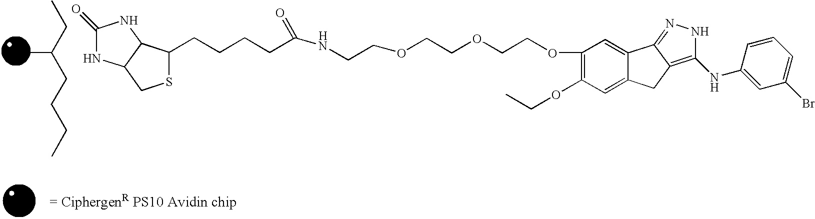 |
| 86 | 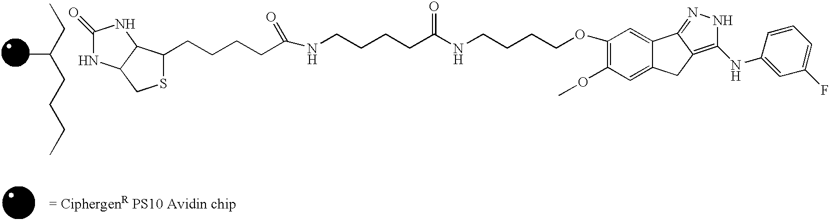 |
| 87 | 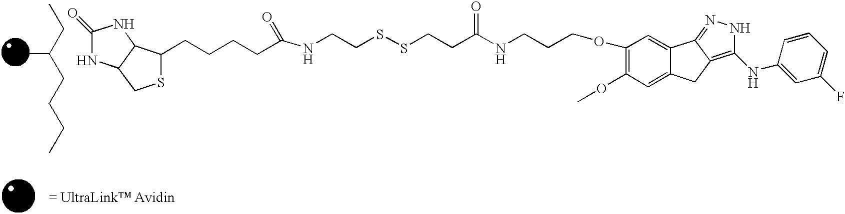 |
| 87a | 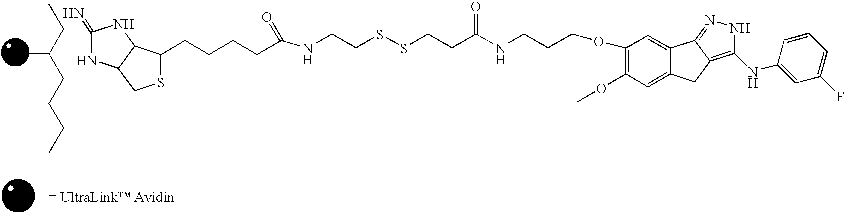 |
| 88 | 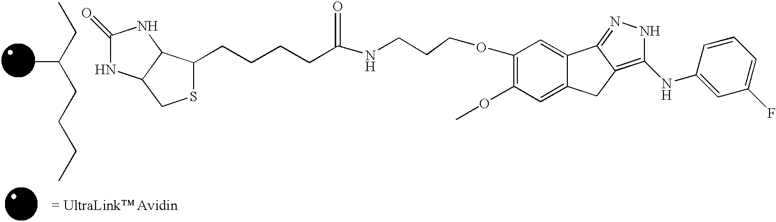 |
| 88a | 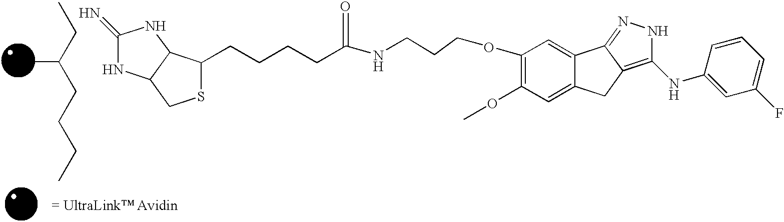 |
| 89 | 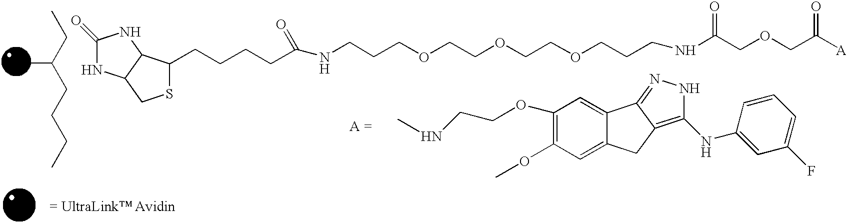 |
| 90 | 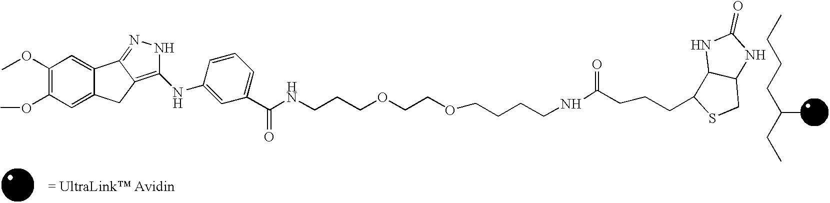 |
| 91 | 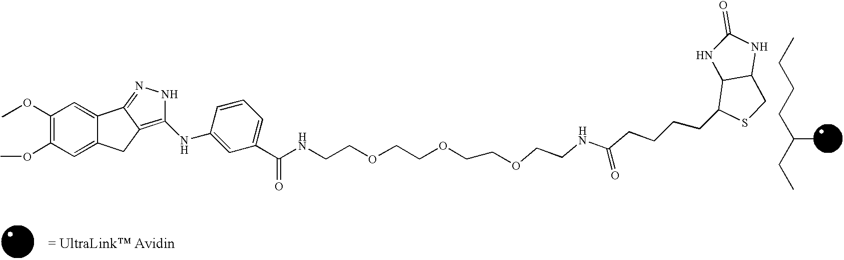 |
| 92 | 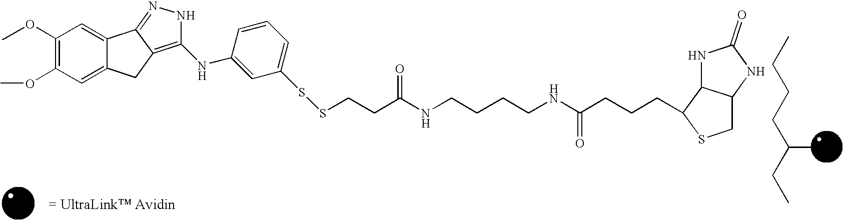 |
| 93 | 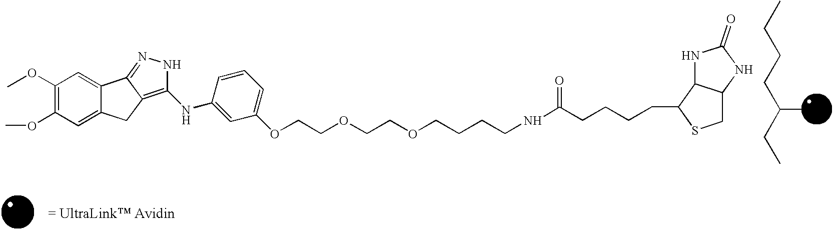 |
| 94 | 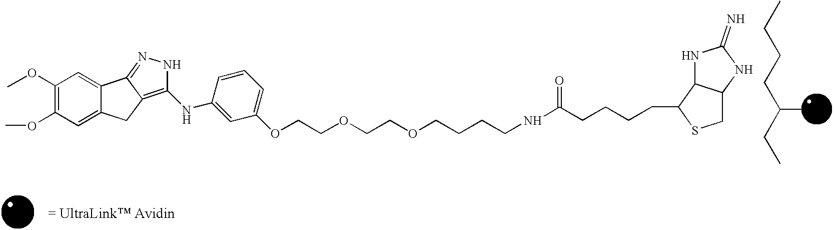 |
| 95 | 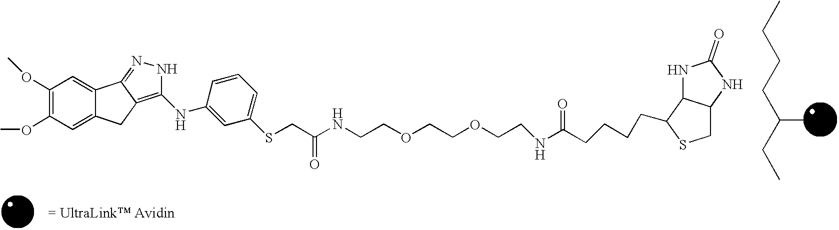 |
| 96 | 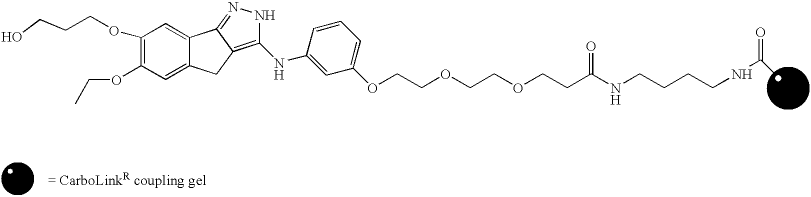 |
| 97 | 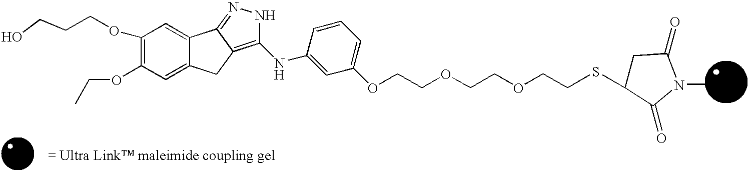 |
| 98 | 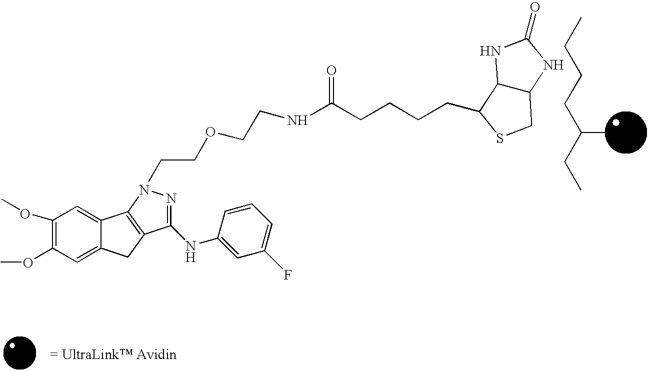 |
| 99 | 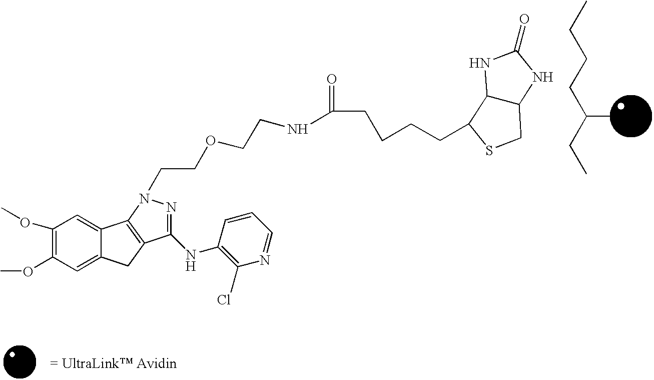 |
| 100 | 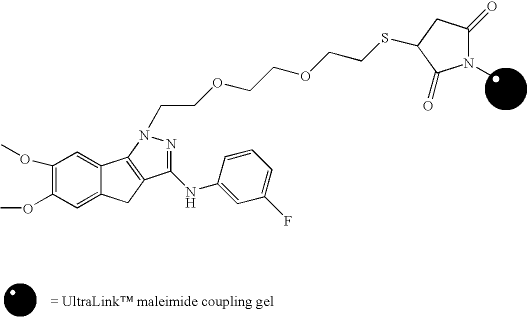 |
| 101 | 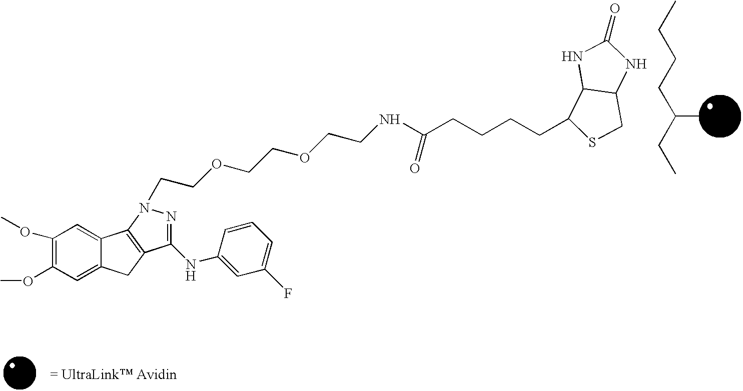 |
| 102 | 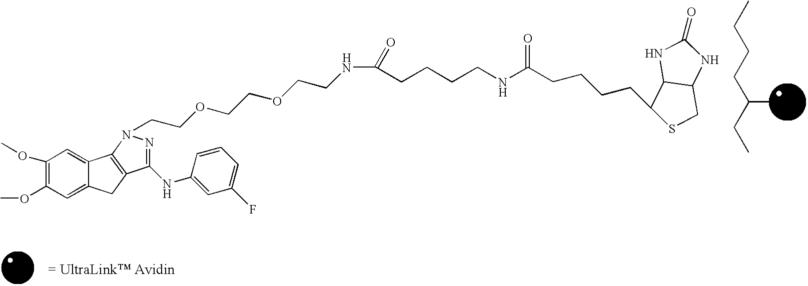 |
| 103 | 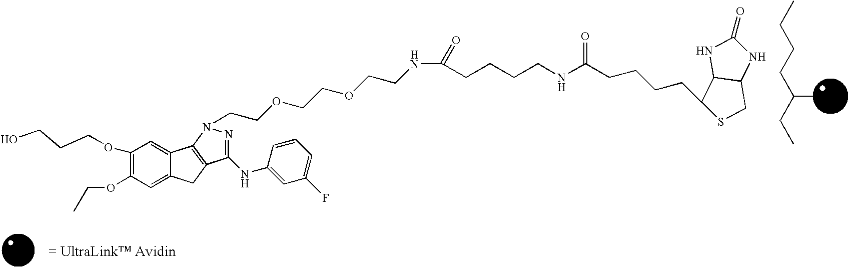 |
| 103a | 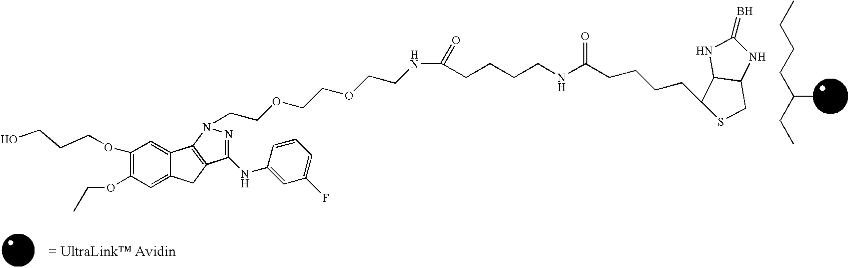 |
| 104 | 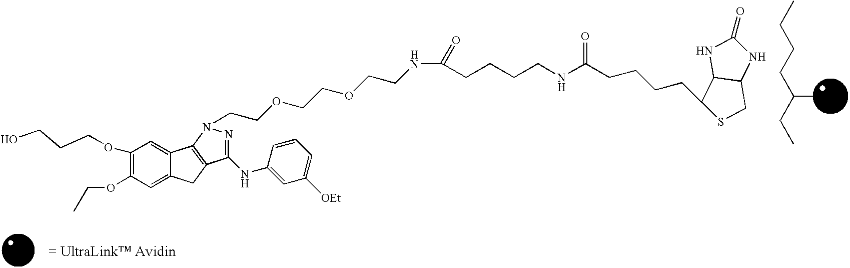 |
| 105 | 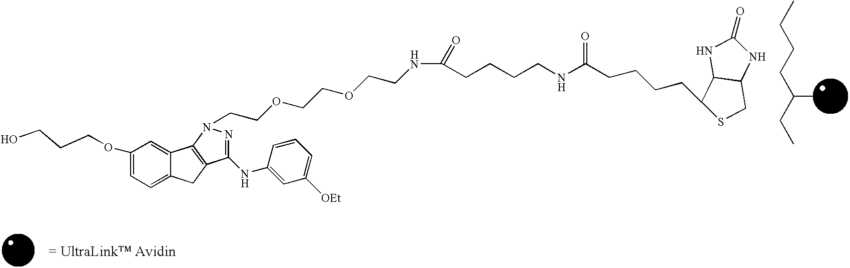 |
| 106 | 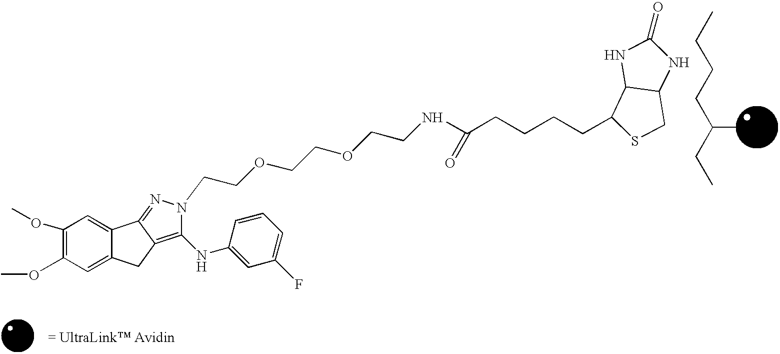 |
| 107 | 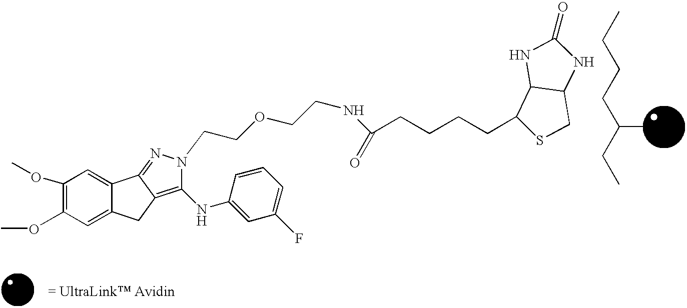 |
| 108 | 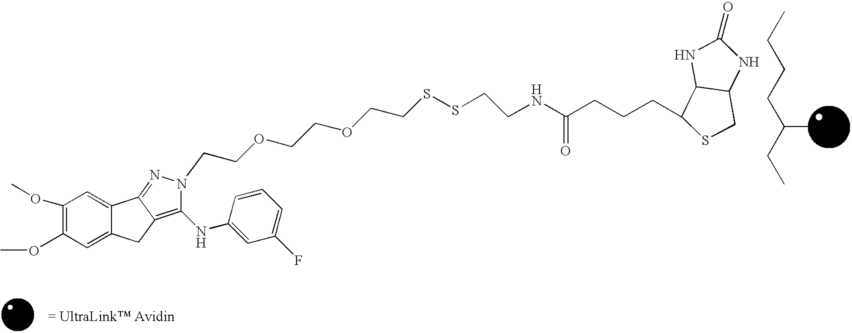 |
| 109 | 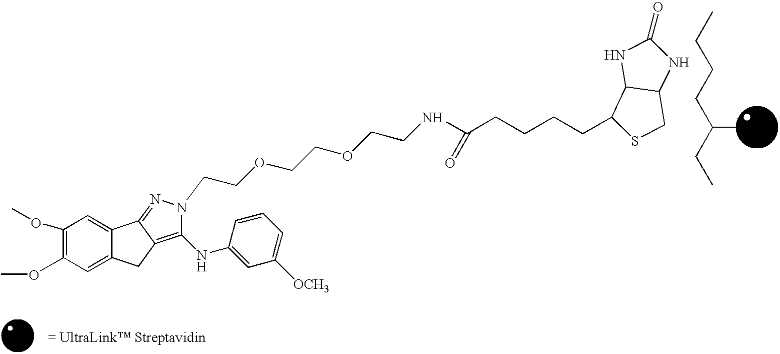 |
| 110 | 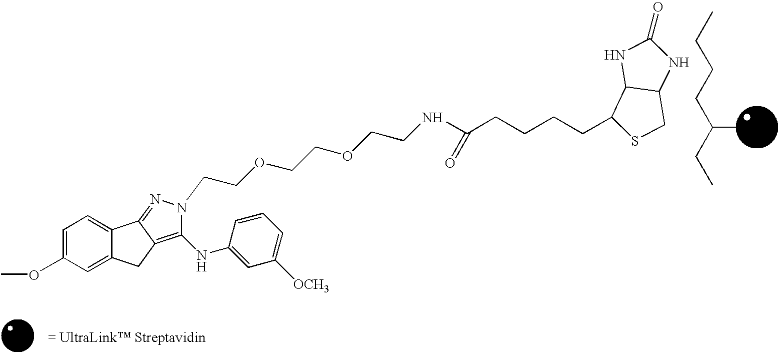 |
| 111 | 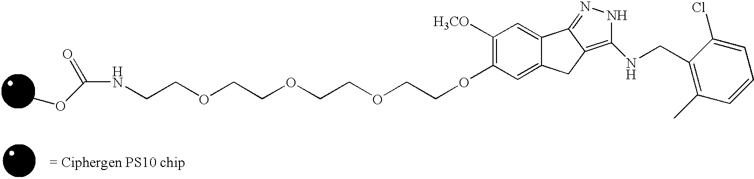 |
| 112 | 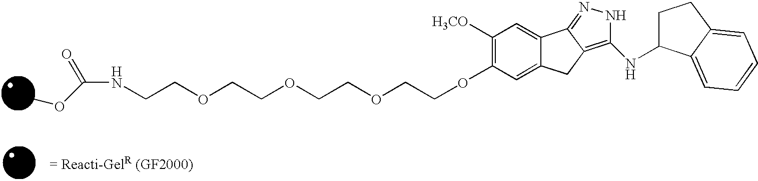 |
| 113 | 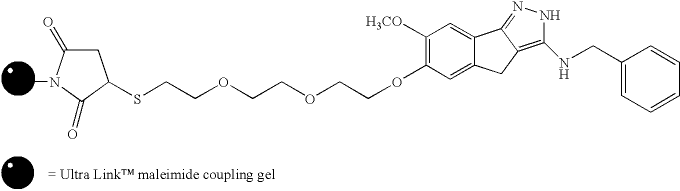 |
| 114 | 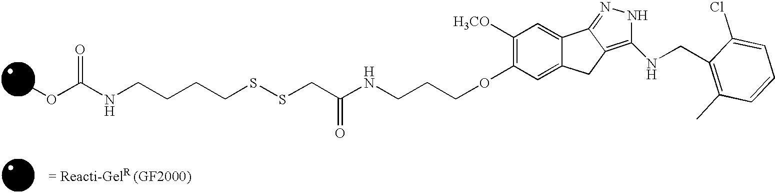 |
| 115 | 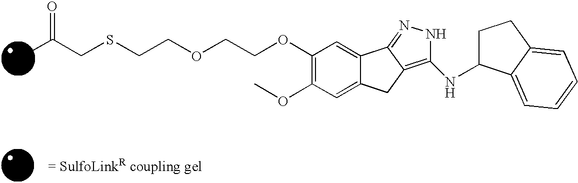 |
| 116 | 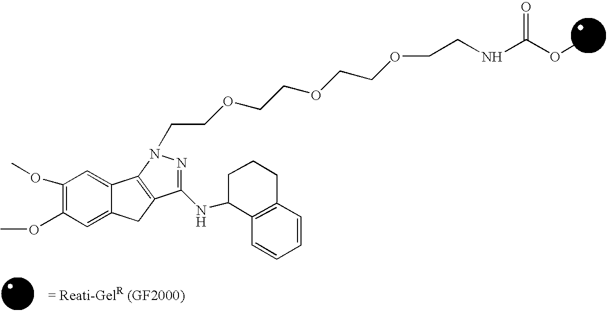 |
| 117 | 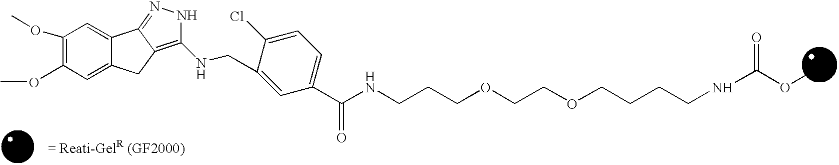 |
| 118 | 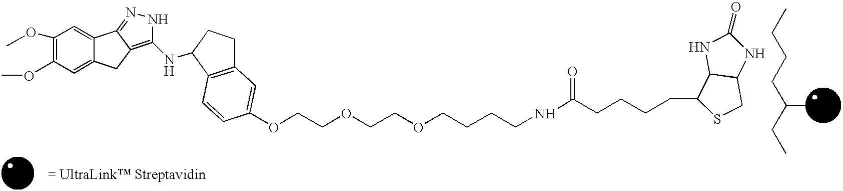 |
| 118a | 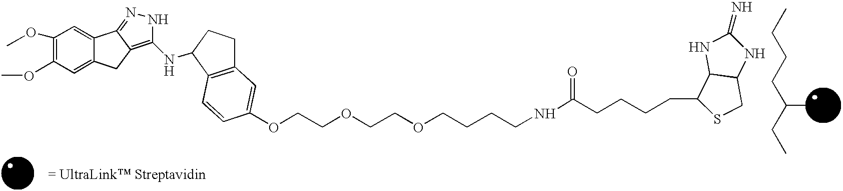 |
| 119 | 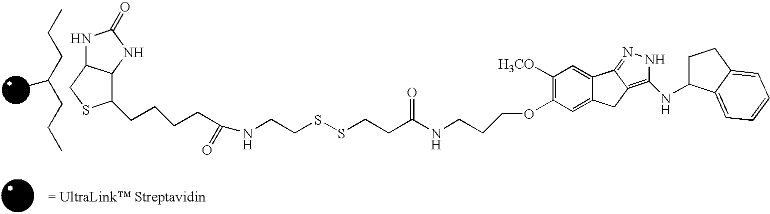 |
| 120 | 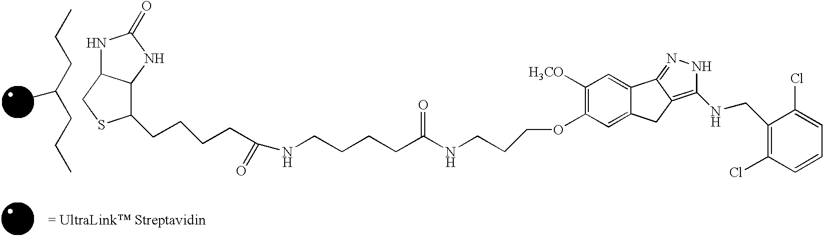 |
| 121 | 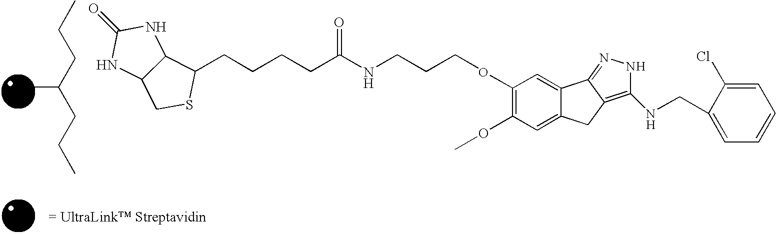 |
| 122 | 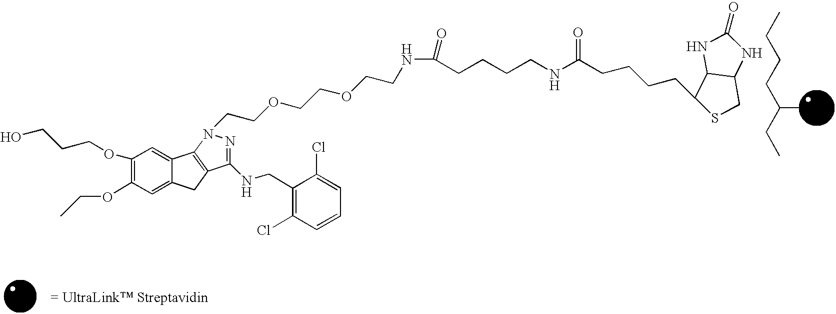 |
| 123 | 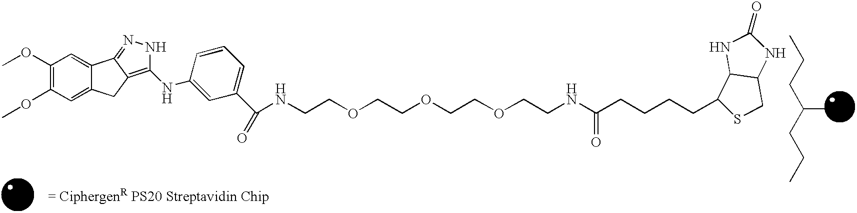 |
| 124 | 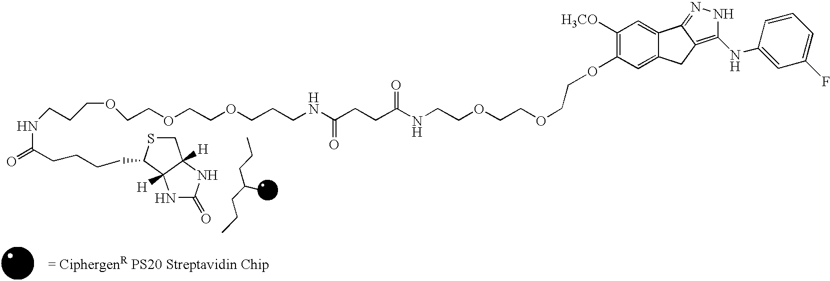 |

The following examples are given for the purpose of illustrating various synthetic methods for compounds of the invention and are not meant to limit the present invention in any fashion.

Abbreviations

Abbreviations used in the specification, particularly the Examples which follow, are as follows:
aq.=Aqueous
DCM=Dichloromethane
DMF=N,N-Dimethylformamide
DMSO=Dimethylsulfoxide
EtOAc=Ethyl acetate
HPLC=High Pressure Liquid Chromatography
LHMDS=Lithium hexamethyldisilylamide
MeOH=Methanol
TFA=Trifluoroacetic Acid
THF=Tetrahydrofuran
TLC=Thin Layer Chromatography
Tris=Tris(hydroxymethyl)aminomethane

### SPECIFIC EXAMPLES

The following examples are given for the purpose of illustrating various synthetic methods for compounds of the invention and are not meant to limit the present invention in any fashion.

### Example 1

### (6-{2-[2-(2-Amino-ethoxy)-ethoxy]-ethoxy}-7-methoxy-2,4-dihydro-indeno[1,2-c]pyrazole-3-yl)-(3-fluoro-phenyl)-amine

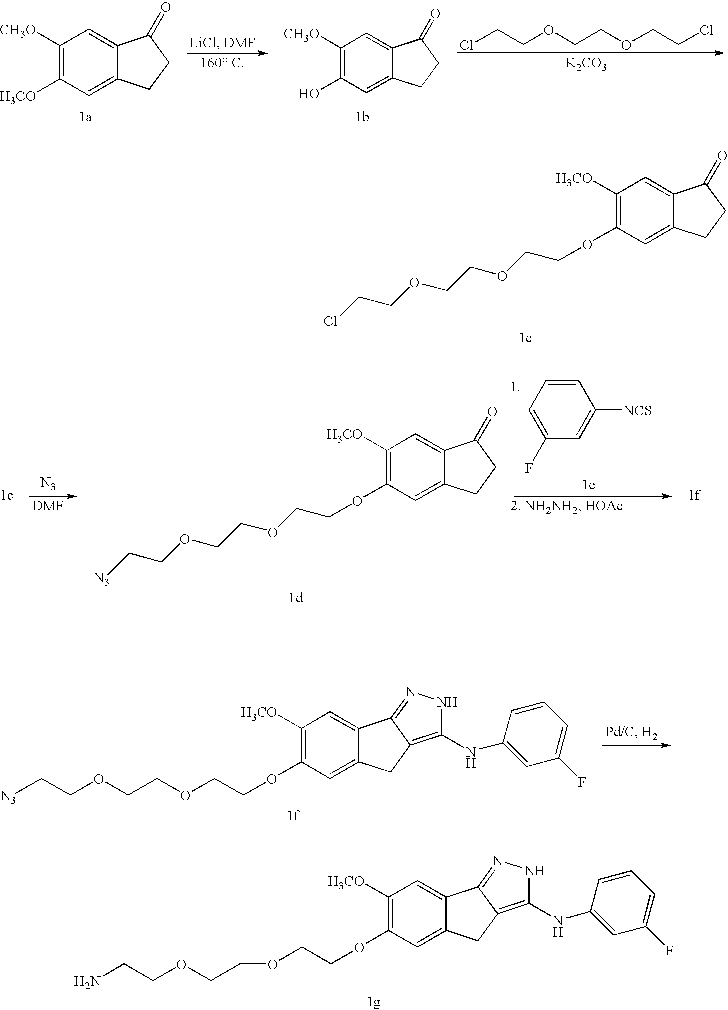

A mixture of 5,6 dimethoxyindan-1-one Compound 1a (25 g, 0.13 mol), LiCl (20 g, 0.47 mol) in DMF (200 mL) was stirred at 160° C. for 60 h. Water (400 mL) was added and the mixture washed with EtOAc. The aqueous layer was acidified with 2N HCl and extracted with EtOAc (2×300 mL). The organic layer was washed with brine and the solvent was removed in vacuo. The crude material was purified (silica gel, DCM/MeOH, 97/3). The solvent was removed in vacuo to yield Compound 1b as a light yellow solid. MS m/z 179 (M+H)⁺.

Compound 1b is a known compound prepared by demethylation of 5,6 dimethyoxyindan-1-one using KCN/DMSO at 100° C. (J. M. Saa et al., J. Org. Chem. 1992, 57, 589).

A mixture of Compound 1b (5 g, 0.028 mol), 1,2-bis(2-chloroethoxy)ethane (15.7 g, 0.084 mol) and potassium carbonate (15.5 g, 0.11 mol) in DMF (100 mL) was stirred at 50° C. overnight. Water (100 mL) was added and the mixture was extracted with EtOAc. The organic phase was washed sequentially with water and brine, and then dried (Na₂SO₄). The solvent was removed in vacuo. The crude material was purified (silica gel, column chromatography, CH₂Cl₂/MeOH, 98/2) to yield Compound 1c, as a light brown solid. MS m/z 329, 331(M+H)⁺; ¹H NMR (CDCl₃): δ 2.63 (m, 2H), 2.98 9m, 2H), 3.57-3.62 (m, 2H), 3.62-3.78 (m, 4H), 3.82 (s, 3H), 3.90 (m, 2H), 4.20 (m, 2H), 6.90 (s, 1H), 7.15 (s, 1H).

A mixture of Compound 1c, (0.20 g, 0.0006 mol) and sodium azide (0.1 g, 0.0012 mol) in DMF (4 mL) was heated at 60° C. for 3 d. Water was added and the mixture was extracted with EtOAc. The organic phase was washed with water, brine and dried (Na₂SO₄). The mixture was concentrated in vacuo to yield Compound 1d. MS m/z 336 (M+H)⁺.

To a mixture of Compound 1d (0.230 g, 0.0007 mol) and 3-fluoro-phenyl isothiocyanate (Compound 1e) (0.1 mL, 0.00083 mol) in THF (3 mL) was added LiHMDS (0.082 mL, 0.00082 mol) dropwise at rt with stirring. After stirring overnight, hydrazine (0.050 mL, 0.00166 mol) and acetic acid (0.075 mL, 0.00125 mol) were added to the reaction mixture, and the mixture was heated to 75° C. for 2 h. Water (10 mL) was added to the mixture, and the aqueous mixture was extracted with CH₂Cl₂. The organic layers were combined and sequentially washed with a solution of NaHCO₃ (aq), water and brine, then dried (Na₂SO₄), and solvent was removed in vacuo. The residue was purified by reverse phase HPLC (CH₃CN/H₂O) to yield Compound 1f as a TFA salt. MS m/z 469 (M+H)⁺; ¹H NMR (DMSO-d₆): δ 3.35(m, 4H); 3.60(m, 6H), 3.75(m, 2H), 3.80(s, 3H), 4.21(m, 2H), 6.50(t, 1H), 6.94(d, 1H), 7.08-7.12(m, 4H), 8.75 (br s, 1H).

A mixture of Compound 1f (0.020 g) and palladium on carbon (0.013 g) in MeOH (2.5 mL) was hydrogenated under 1 atm of H₂ with stirring at rt for 0.5 h. The catalyst was removed by filtration and the resulting filtrate was concentrated in vacuo to yield the title compound, Compound 1g. MS m/z 443 (M+H)⁺.

### Example 1a

Preparation of 200 mM NaHCO₃ Buffer Coupling Solution, Buffer A: NaHCO₃ (1.68 g, 0.02 mol) and NaCl (1.17 g, 0.02 mol) were dissolved in water (100 mL) to give 200 mM of a NaHCO₃ buffer solution.

### Example 2

### Immobilized (6-{2-[2-(2-Amino-ethoxy)-ethoxy]-ethoxy}-7-methoxy-2,4-dihydro-indeno[1,2-c]pyrazole-3-yl)-(3-fluoro-phenyl)-amine on agarose gel, Cpd 2

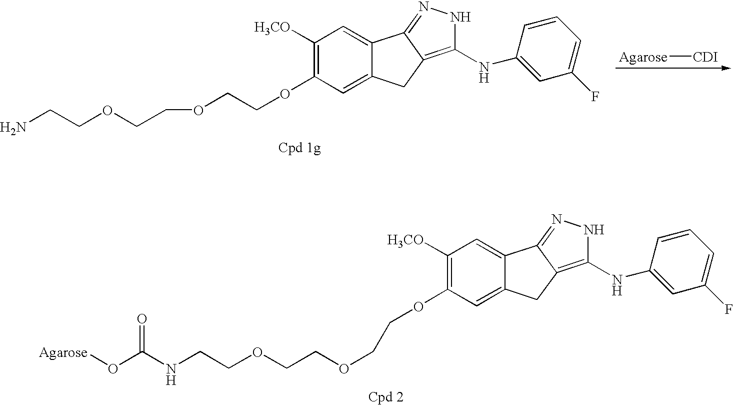

A mixture of Compound 1g, (0.020 g, 0.045 mmol) in 0.2 mL MeOH, Agarose gel-CDI activated (Pierce Reacti-Gel), and Buffer A (3 mL) was agitated at 4° C. overnight. The mixture was then quenched with hydroxyethylamine (3 mL, 0.2 M) in Buffer A and stirred for 2 h. The mix was sequentially washed with Buffer A (6×) and then water (6×). The loading was measured to be 7 μmol/mL per bead using HPLC analysis and 1-indanol as an internal standard.

### Example 3

### Immobilized (6-{2-[2-(2-Amino-ethoxy)-ethoxy]-ethoxy}-7-methoxy-2,4-dihydro-indeno[1,2-c]pyrazole-3-yl)-(3-fluoro-phenyl)-amine on agarose gel—GF2000, Cpd 7

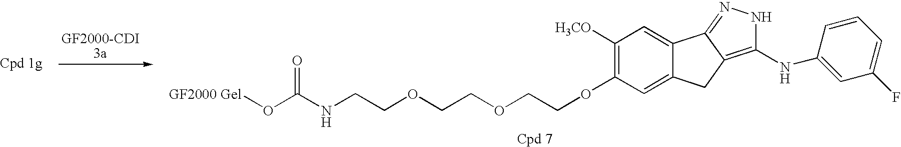

A mixture of Compound 2 (0.086 g, 0.18 mmol) in 0.4 mL MeOH, Agarose gel (Pierce CDI-activated Trisacryl GF-2000), and Buffer A (6 mL) was agitated at 4° C. overnight. The mixture was washed with additional Buffer A and then was treated with 0.2 M hydroxylethylamine (6 mL) and stirred for 2 h. The gel was thoroughly washed with buffer solution (6×), and then water (3×). The loading was determined to be 72 μmol/mL/bead using HPLC analysis and 1-indanol as an internal standard.

### Example 4

### Immobilized (6-{2-[2-(2-Amino-ethoxy)-ethoxy]-ethoxy}-7-methoxy-2,4-dihydro-indeno[1,2-c]pyrazole-3-yl)-(3-fluoro-phenyl)-amine on Ciphergen protein PS-10 Chips, Cpd 6

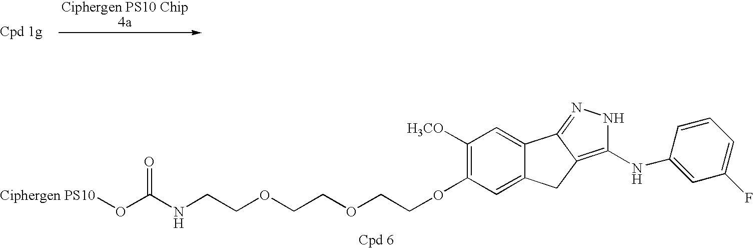

To a solution of Compound 2 (0.02 g, 0.18 mmol) in DMF (2 mL) was added Buffer A (2 mL). The mixture was placed in a 8×12 platform containing Ciphergen PS10 chips (Compound 4a) (100 μL per well) and the platform was agitated for 3 h at rt. The chip was washed with Buffer A (3×), quenched with Tris-solution (0.2 mM), and then washed with Buffer A (6×) and water (3×).

### Example 5

### (7-{3-[2-(2-Amino-ethoxy)-ethoxy]-propoxy}-6-ethoxy-2,4-dihydro-indeno[1,2-c]pyrazol-3-yl)-(3-ethoxy-phenyl)-amine

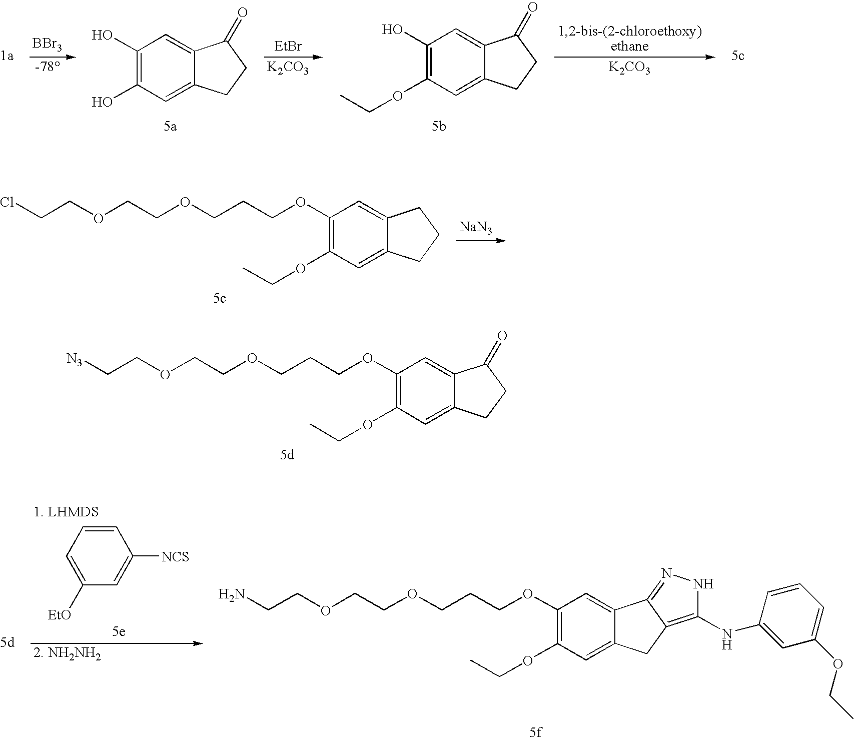

A solution of Compound 1a (19 g, 0.094 mol) in 200 mL of methylene chloride was cooled to −78° C. using a dry ice/i-PrOH bath. A solution of BBr₃ in CH₂Cl₂ (200 mL, 0.2 mol) was added dropwise. The resulting solution was stirred at −78° C. for 1 h, at which time the temperature was allowed to warm to 0° C. with stirring for an additional 1 h. The mixture was then cooled back to −78° C. and quenched with MeOH (50 mL). The solution was concentrated under reduced pressure to dryness. The resulting solid was dissolved in MeOH (50 mL) and concentrated under reduced pressure two more times. The red solid, Compound 5a, was used in the subsequent reaction without further purification. MS m/z 165 (M+H)⁺.

A mixture of Compound 5a, (2.0 g, 0.00122 mol), potassium carbonate (4.2 g, 0.0305 mol) and ethyl bromide (0.911 mL, 0.0122 mol) in DMF (20 mL) was stirred at rt for 12 h. The reaction mixture was diluted with EtOAc, washed with water, and dried (MgSO₄), filtered, and concentrated in vacuo. The residue was purified by column chromatography (silica gel, 3/1 hexanes/EtOAc) to yield Compound 5b (5-ethoxy-6-hydroxy-indan-1-one).

A mixture Compound 5b, (1.5 g, 0.0078 mol), 1,2-bis-(2-chloroethoxy)ethane (8.76 g, 0.047 mol), and potassium carbonate (2.15 g, 0.0156 mol) in DMF (20 mL) was stirred at 50° C. for 3 d, followed by the addition of water. The mixture was extracted with EtOAc (2×). The combined organic extracts were concentrated and the 1,2-bis(2-chloroethoxy)ethane was removed in vacuo to yield a residue, Compound 5c, which was used in the subsequent reaction. MS m/z 343 and 345 (M+H)⁺.

A mixture of Compound 5c (2.15 g, 0.006 mol), sodium azide (4.08 g, 0.063 mol) in DMF (15 mL) was heated to 60° C. for 3 d, followed by the addition of water. The aqueous mixture was extracted with EtOAc, and the organic phase was separated and washed sequentially with water and brine, dried (Na₂SO₄), filtered and concentrated under reduced pressure to yield Compound 5d. MS m/z 350 (M+H)⁺.

A solution of Compound 5d (2.26 g, 0.0065 mol), 3-ethoxy-phenyl isothiocyanate, Compound 5e, (1.09 g, 0.0065 mol) in THF (25 mL) was added LHMDS (7.8 mL, 0.0078 mol) dropwise at rt with stirring. The reaction mixture was stirred overnight, and then acetic acid (0.5 mL, 0.0125 mol) and hydrazine (0.4 mL, 0.0125 mol) were added. The reaction mixture was heated at 75° C. for 5 h, then poured into a solution of NaHCO₃ (aq) (10 mL), and extracted with EtOAc. The organic layers were combined and washed sequentially with NaHCO₃ solution (aq), water, and brine, then dried (Na₂SO₄) and filtered. The filtrate was concentrated in vacuo. The residue was purified by reverse phase HPLC to yield the title compound, Compound 5f as a TFA salt. MS m/z 483 (M+H)⁺; ¹H NMR (DMSO-d₆): δ 1.31-1.35 (m, 6H), 2.97-2.99 (m, 2H), 3.49 (s, 2H), 3.59-3.65 (m, 4H), 3.66-3.67 (m, 2H), 3.77-3.79 (m, 2H), 3.95-3.99 (m, 2H), 4.04-4.08 (m, 2H), 6.43-4.45 (m, 2H), 6.71-6.75 doublet and singlet; 7.13-7.16 (t, 1H), 7.22 (s, 1H), 7.27 (1H), 7.90 (br. s, 2H).

### Example 6

### Immobilized (7-{3-[2-(2-Amino-ethoxy)-ethoxy]-propoxy}-6-ethoxy-2,4-dihydro-indeno[1,2-c]pyrazol-3-yl)-(3-ethoxy-phenyl)-amine on Ciphergen protein PS-10 Chips, Cpd 3

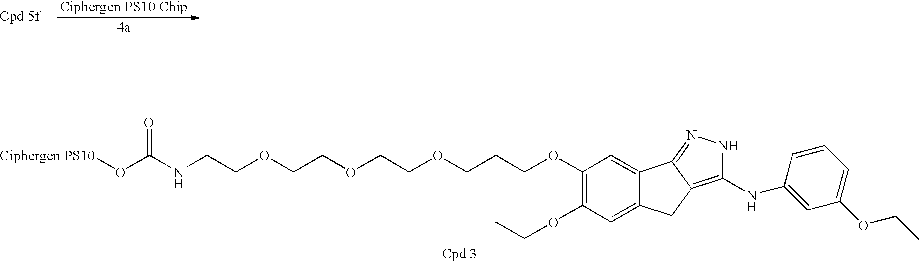

A mixture of Compound 5f, (0.0022 g, 0.0037 mmol), DMF (0.3 mL) and Buffer A (0.3 mL), was added to a 8×12 platform containing Ciphergen PS10 chips (35 μL per well) and the platform was agitated for 4 h at rt. The chip strip was washed with Buffer A (3×) and then quenched with 50 μL of a solution of hydroxyethylamine (80 μL) in NaHCO₃ buffer solution (0.8 mL) for 1 h and then was washed with Buffer A (3×) and water (5×).

### Example 7

### (3-{2-[2-(2-Amino-ethoxy)-ethoxy]-ethoxy}-phenyl)-(6,7-dimethoxy-2,4-dihydro-indeno[1,2-c]pyrazol-3-yl)-amine

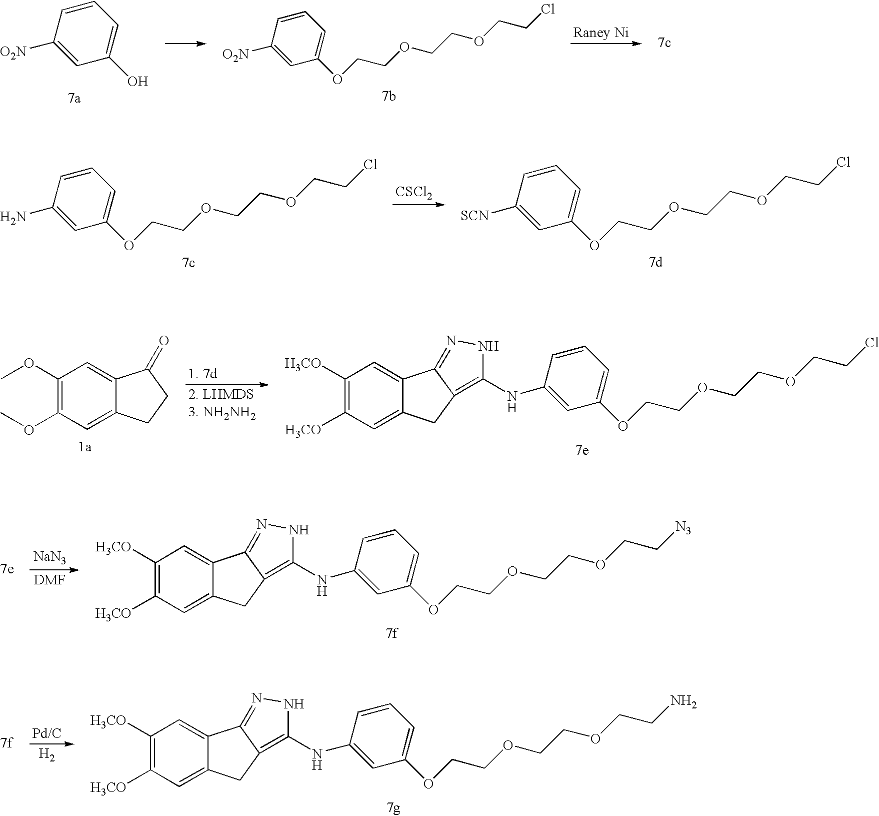

A mixture of 3-nitrophenol, Compound 7a, (1.51 g, 0.011 mmol), 1,2-bis(2-chloroethoxy)ethane (7.26 g, 0.039 mol) and potassium carbonate (3.60 g, 0.026 mol) in DMF (100 mL) was stirred at 50° C. overnight. The reaction mixture was poured into water (100 mL) and then extracted with EtOAc, washed sequentially with water 3×) and brine, and dried (NaSO₄) and filtered. The filtrate was concentrated in vacuo. Excess 1,2-bis(2-chloroethoxy)ethane was removed by distillation (pot temperature up to 95° C. under 0.1 mmHg) to yield Compound 7b. ¹H NMR(CDCl₃): δ 3.6-3.8 (m, 8H), 3.78-3.8 (m, 2H), 4.2 (m, 2H), 7.25 (m, 1H), 7.4 (m, 1H), 7.78 (m, 1H), 7.9 (d, 1H).

A mixture of Compound 7b (2 g, 0.0069 mol) and Raney-nickel (1 g) in MeOH (20 mL) was heated at 65° C., followed by the dropwise addition of hydrazine (0.2 mL) over 10 min. Heating was continued for additional 10 min. The catalyst was filtered off and the solution was concentrated in vacuo to yield Compound 7c. MS m/z 260 and 262 (M+H)⁺; ¹H NMR (CDCl₃): δ 3.6-3.8 (m, 10H), 4.02-4.1 (m, 2H), 6.2-6.32 (m, 3H), 7-7.07 (t, 1H).

A mixture of thiophosgene (0.65 g, 0.0056 mol) and water (5 mL) was stirred at 0° C., followed by the dropwise addition of a solution of Compound 7c (0.5 g, 0.0019 mol) in chloroform (5 mL). The resulting mixture was stirred for 1 h and the organic layer was separated and washed with water and brine. The organic phase was dried and concentrated in vacuo to yield Compound 7d. MS m/z 302 (M+H)⁺.

To a solution of Compound 1a (0.7 g, 0.0036 mol) and Compound 7d (1.30 g, 0.004 mol) in THF (5 mL) was added LHMDS in THF (1M) (3.75 mL, 0.00375 mol) dropwise at rt with stirring. The reaction mixture was stirred overnight and hydrazine (0.12 mL, 0.00375 mol) and acetic acid (0.264 mL, 0.0044 mol) were added to the reaction mixture. The reaction mixture was then heated at 75° C. for 2 h. To the resulting mixture was first added water (10 mL) followed by extraction with CH₂Cl₂. The organic layers were combined and washed sequentially with NaHCO₃ solution (aq), water, and brine. The organic phase was then dried (Na₂SO₄), filtered and the concentrated in vacuo. The residue was purified by reverse phase HPLC to yield Compound 7e as an off-white powder. MS m/z 474, 466 (M+H)⁺.

A mixture of Compound 7e (0.90 g, 0.0019 mol) and sodium azide (1.23 g, 0.019 mol) in DMF (10 mL) was heated at 55° C. for 3 d. The reaction mixture was poured into water and then extracted with EtOAc. The organic extracts were washed with water and brine, dried (Na₂SO₄), filtered and concentrated in vacuo to yield Compound 7f. MS m/z 481 (M+H)⁺.

A mixture of Compound 7f (0.042 g, 0.09 mmol) and palladium on carbon (0.010 g) in MeOH (5 mL) was hydrogenated under 1 atm of H₂ with stirring at rt for 2 h. The catalyst was removed by filtration, and the filtrate was concentrated to yield the title compound, Compound 7g. MS m/z 455 (M+H)⁺.

### Example 8

### Immobilized (3-{2-[2-(2-Amion-ethoxy)-ethoxy]-ethoxy}-phenyl)-(6,7-dimethoxy-2,4-dihydro-indeno[1,2-c]pyrazol-3-yl)-amine on Ciphergen protein PS-10 Chips, Cpd 5

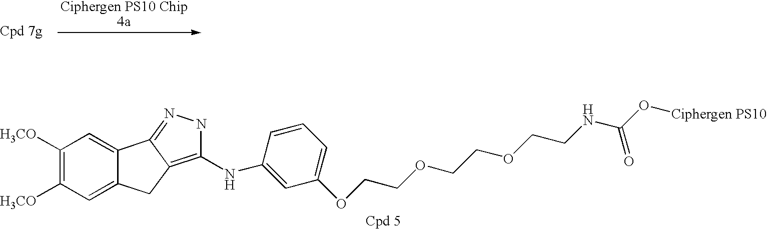

A mixture Compound 7f (0.042 g, 0.09 mmol) in DMF (0.3 mL) and buffer Buffer A (0.3 mL) was added to a 8×12 platform containing Ciphergen PS10 chips (35 μL per well) and the chip platform was agitated for 4 h at rt. The chip strip was washed with the buffer solution (3×) and then quenched with 50 μL of ethanolamine solution (80 μL in 0.8 mL Buffer A) for 1 h. The chip with compound, Compound 5, was washed with buffer solution (3×) and water (5×).

### Example 9

### (1-{2-[2-Amino-ethoxy)ethoxy]-ethyl}-6,7-dimethoxy-1,4-dihydro-indeno[1,2-c]pyrazol)-(3-fluoro-phenyl)-amine, and (2-{2-[2-Amino-ethoxy)ethoxy]-ethyl}-6,7-dimethoxy-2,4-dihydro-indeno[1,2-c]pyrazol)-(3-fluoro-phenyl)-amine

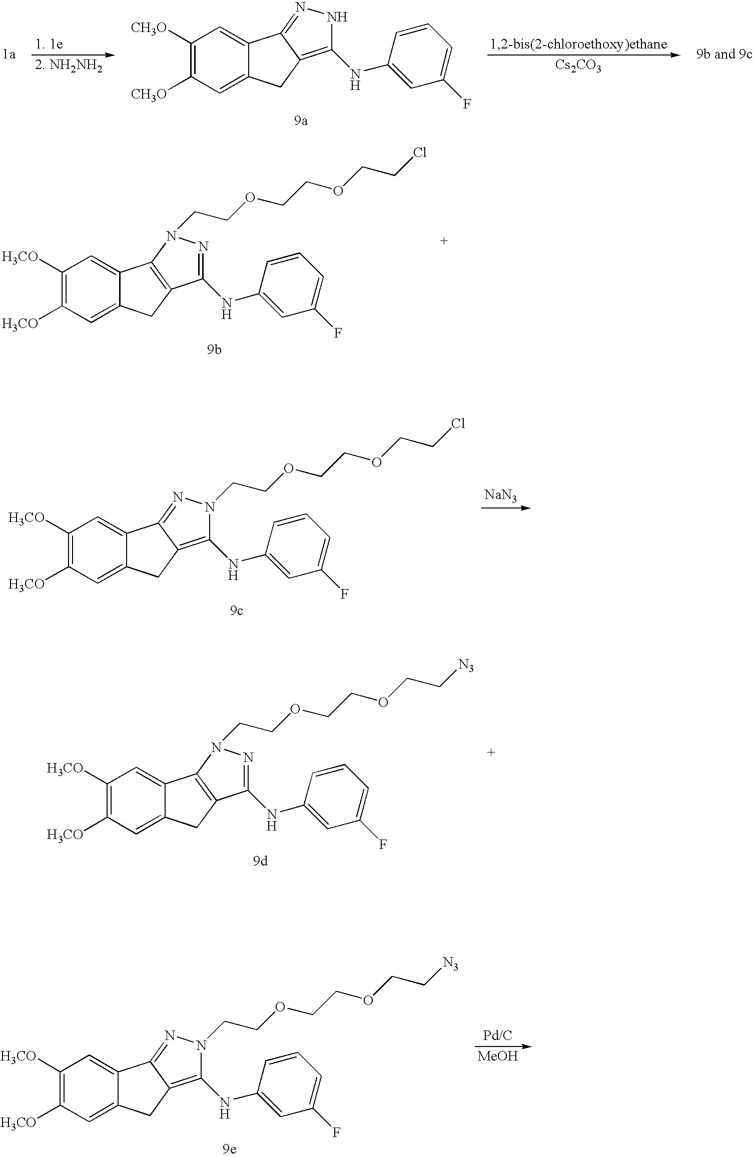

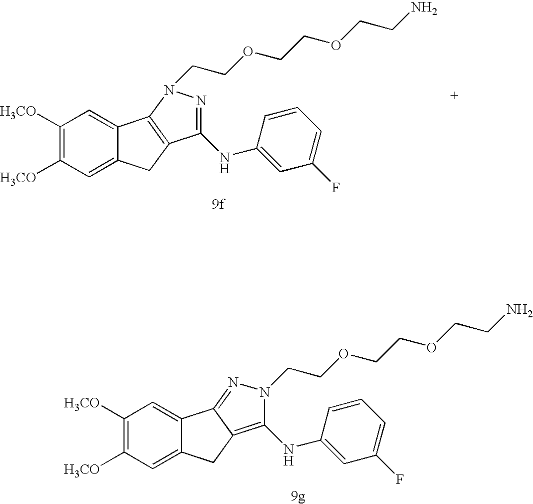

Compound 1a (3.0 g, 0.0154 mol) and Compound 1e (2.4 g, 0.0157 mol) in THF (3 mL) was added to LHMDS (15.4 mL, 0.0154 mol) dropwise at rt with stirring. The mixture was stirred for 12 h, at which time hydrazine (0.75 mL, 0.0154 mol) and acetic acid (0.96 mL) were added. The mixture was heated at reflux for 24 h, then added to water (30 mL) and extracted with CH₂Cl₂. The organic layers were combined, washed sequentially with NaHCO₃ solution (aq), water, and brine, then dried (Na₂SO₄), filtered and concentrated in vacuo. The residue was dissolved in hot CH₃CN and one equivalent of HCl etherate was added to afford a precipitate. The precipitate was dissolved in CH₃CN, decolorized with charcoal, re-crystallized, collected and dried under vacuum at rt to give Compound 9a as an off white solid. MS m/z 326 (M+H)⁺; ¹H NMR (DMSO-d₆): δ 3.44 (s, 2H), 3.80 (s, 3H), 3.81 (s, 3H), 6.58 (t, 1H), 6.85 (d, 1H), 7.1 (d, 1H), 7.21 (s, 1H), 7.23 (s, 1H), 7.3 (m, 1H), 9.2 (br s, 1H).

A mixture of Compound 9a (1.4 g, 0.0038 mol), 1,2-bis(2-chloroethoxy)ethane (8.8 g, 0.047 mol) and cesium carbonate (6 g, 0.018 mol) in DMF (30 mL) was stirred at rt for 2 d. Water (100 mL) was added and the aqueous phase was extracted with EtOAc. The organic phase was washed with water, dried (Na₂SO₄), filtered and concentrated in vacuo. The residue was purified using Gilson HPLC reverse phase chromatography (CH₃CN/H₂O with TFA (0.1%)) to yield a mixture of two isomers, Compound 9b and 9c. A small-scale purification was performed to separate Compounds 9b and 9c for identification. MS m/z 475 (M+H)⁺; Compound 9b: ¹H NMR (DMSO-d₆): δ 3.41-3.58 (m, 10H), 3.8 (s, 3H), 3.97 (s, 3H), 3.90 (m, 2H), 6.5 (t, 1H), 7.02 (d, 1H), 7.15 (d of d, 1H), 7.16 (s, 1H), 7.3 (s, 1H), 7.35 (d, 1H); Compound 9c: ¹H NMR (DMSO-d₆): δ 3.3 (s, 2H), 3.45 (m, 4H), 3.6 (m, 4H), 3.75-3.8 (two s, 6H and m, 2H), 4.15 (t, 2H); 6.5 (m, 1H), 6.55 (m, 1H), 6.65 (m, 1H), 7.1 (s, 1H), 7.15 (s, 1H), 7.2 (m, 1H), 8.2 (s, NH). The structures were assigned by hetero-multiple-bond correlation (HMBC) experiments. The mixture of Compound 9b and 9c was used for subsequent reactions.

A mixture of Compounds 9b and 9c (0.32 g, 0.68 mmol) and sodium azide (0.045 g, 6.8 mmol) in DMF (25 mL) was heated at 60° C. for 2 d. Water was added to the mixture which was then extracted with EtOAc. The combined organic extracts were washed with water and brine. The organic phase was dried (Na₂SO₄), filtered, and concentrated in vacuo to yield Compounds 9d and 9e as a mixture of two isomers. MS m/z 483 (M+H)⁺.

A mixture of Compounds 9d and 9e (0.04 g, 0.002 mmol) and palladium on carbon (0.012 g) in MeOH (5 mL) was hydrogenated under a H₂ atmosphere (1 atm) with stirring at rt for 2 h. The catalyst was removed and the filtrate was concentrated to yield Compounds 9f and 9g. MS m/z 457 (M+H)⁺.

### Example 10

### Immobilized (1-{2-[2-Amino-ethoxy)ethoxy]-ethyl}-6,7-dimethoxy-1,4-dihydro-indeno[1,2-c]pyrazol)-(3-fluoro-phenyl)-amine and (2-{2-[2-Amino-ethoxy)ethoxy]-ethyl}-6,7-dimethoxy-2,4-dihydro-indeno[1,2-c]pyrazol)-(3-fluoro-phenyl)-amine on Ciphergen PS10, Cpd 4

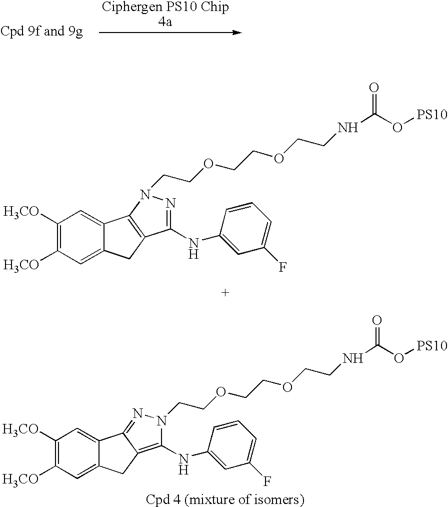

A mixture of Compound 9f and Compound 9g (0.040 g, 0.09 mmol) in DMF (0.3 mL) and buffer solution (0.3 mL) was added into a 8×12 platform containing Ciphergen PS10 chips (35 μL per well) and the chip platform was agitated for 4 h at rt. The chip strip was washed with buffer solution (3×) and then quenched with 50 μL of hydroxyethylamine solution (80 μL in 0.8 mL NaHCO₃ buffer solution) for 1 h and then was washed with buffer solution (3×) and water (5×) to yield Compound 4.

### Example 11

### 3-(6,7-Dimethoxy-2,4-dihydro-indeno[1,2-c]pyrazol-3-ylamino)-N-{-2-[2-(-{2-[5-(2-oxo-dihydro-thieno[3,4-d]imidazol-4-yl)-pentanoyl]-ethoxy}-ethoxy)-ethoxyl]-ethyl}-benzamide, Cpd 1

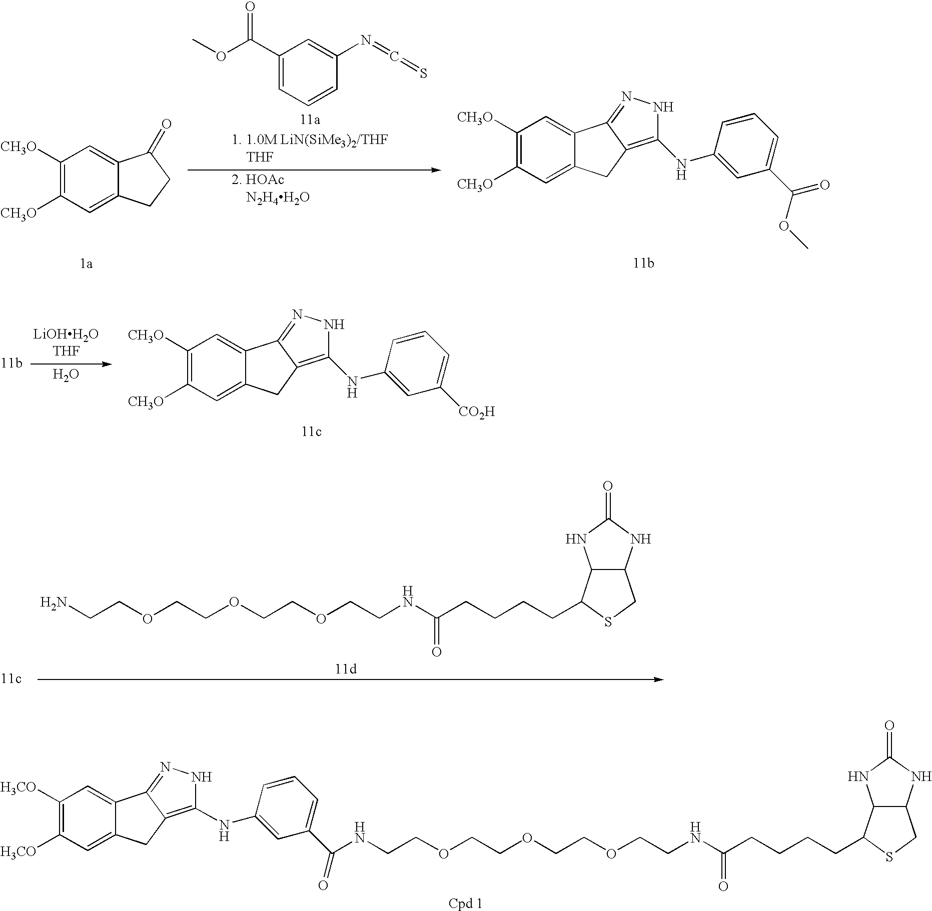

To a flask under argon was added Compound 1a (0.30 g, 1.51 mmol), isothiocyanate Compound 11a (0.30 g, 1.54 mmol), 1.5 mL THF, and 1.54 mL (1.54 mmol, 1.0 M) LHMDS. The reaction mixture was stirred for 5 min, then glacial acetic acid (0.095 mL, 1.665 mmol) and hydrazine hydrate (0.079 mL, 1.54 mmol) were added. The reaction mixture was heated at 70° C. for 16 h, and then 1 mL of water was added and the solution was filtered through a Varian Cartridge Elut 1003. The cartridge was washed with 8.5 mL methylene chloride and the elulent was evaporated. The residue was purified by reverse phase chromatography to give 0.10 g of Compound 11 b as a TFA salt.

To a flask under argon was added 0.16 g (0.33 mmol) of Compound 11b, THF (4.5 mL), 1 mL H₂O, and 0.043 g (1.0 mmol) of lithium hydroxide monohydrate. The reaction mixture was stirred for 2 d at rt. The solvent was evaporated, water and a ½ drop of TFA were added, and the sample was purified by reverse phase chromatography to give 0.06 g of Compound 11c as its TFA salt.

Compound 11c (20 mg, 0.043 mmol), Compound 11d (34 mg, 0.09 mmol) (biotin-PEO-LC-amine (5-(2-oxo-hexahydro-thieno[3,4-d]imidazol-4-yl)-pentanoic acid (2-{2-[2-(2-amino-ethoxy)-ethoxy]-ethoxy}-ethyl)-amide, from Pierce), EDC (15.5 mg, 0.09 mmol), HOBt (11 mg, 0.09 mmol), and DIEA (0.016 mL, 0.09 mmol) were stirred together in a minimal volume of DMF. The reaction was stirred at rt overnight, then quenched with water and extracted with EtOAc. The organic phase was separated, dried (Na₂SO₄), filtered, and concentrated in vacuo. The residue was purified by reverse phase HPLC to give Compound 1. MS m/z 752 (M+H)⁺; ¹H NMR (CDCl₃) at 60° C.: δ 1.5 (m, 2H), 1.72 (m, 4H), 2.11 (t, 2H), 2.85 (dd, 2H), 3.10 (q, 1H), 3.30 (m, 2H), 3.67 (m, 16H), 3.91 (s, 3H), 3.93 (s, 3H), 4.30 (m, 1H), 4.55 (m, 1H), 7.07 (s, 1H), 7.30 (m, 2H), 7.52 (t, 1H), 7.69 (m, 2H), 9.44 (s, 1H).

### Example 12

### Immobilized N-[2-(2-{2-[3-(3-Fluoro-phenylamino)-7-methoxy-2,4-dihydro-indeno[1,2-c]pyrazol-6-yloxy]-ethoxy}-ethoxy)-ethyl]-N′-{3-[2-(2-{3-[5-(2-oxo-hexahydro-thieno[3,4-d]imidazol-6-yl)-pentanoylamino]-propoxy}-ethoxy)-ethoxy]-propyl}-succinamide on Streptavidin-coated Ciphergen PS20 chip, Cpd 124

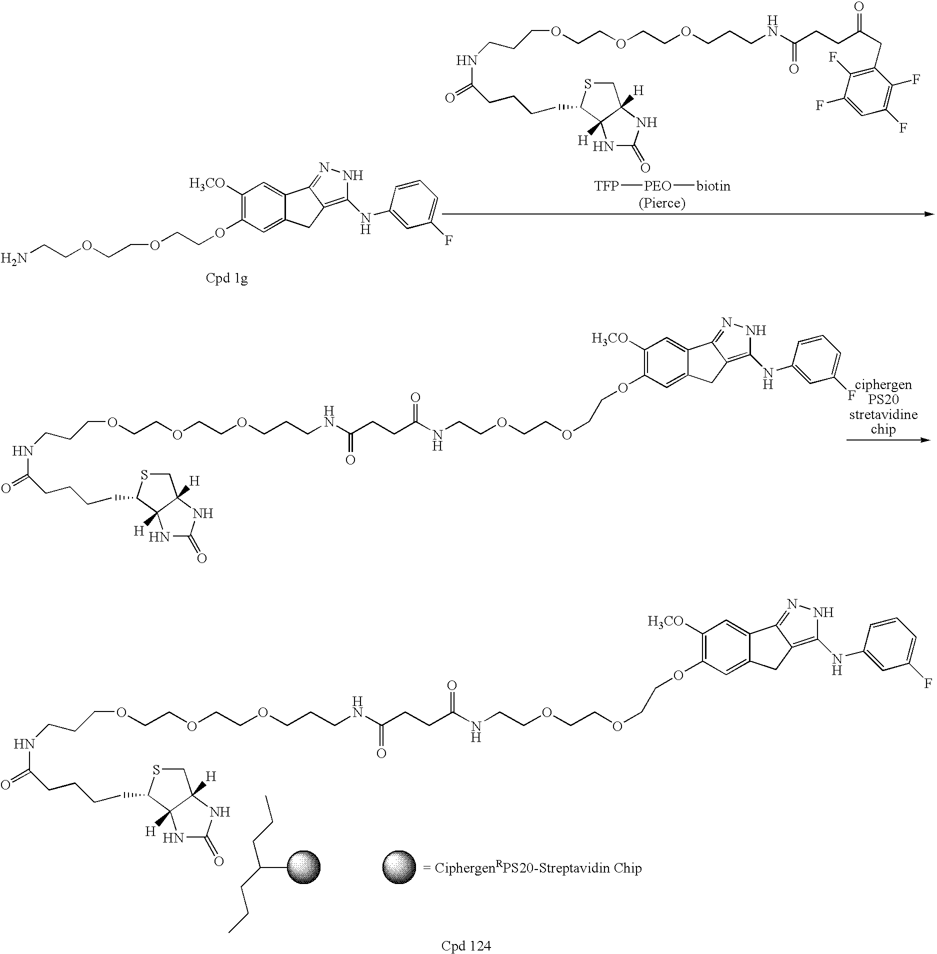

A mixture of Compound 1g, (0.051 g, 0.109 mmol) and 130 mg of TFP-PEO biotin (obtained from Pierce) in 1 mL DMF and 0.1 mL of Di EA was stirred at room temperature for overnight. The mixture was then diluted with CH₃CN and H₂O (1:1) and then was lyophilized to dryness. The obtained crude was purified by Gilson C-18 reverse phase column chromatography using CH₃CN—H₂O containing 0.1% TFA. The desired fractions were collected and lyophilized to give a colorless solid as a TFA salt. MS m/z 972 (M+H)⁺.

The obtained compound was dissolved in DMF and was incubated with Ciphergen PS20®-Streptavidine chip at room temperature. The chip was washed with DMF and distilled water and then air-dried to give Cpd 124.

2. Biological Application

2.A Identification of a Biologically Relevant Target

As used herein, “a core structure of a compound of formula 1” (also referred to as “a core structure compound of a compound of formula 1”) refers to the portion of the compound of formula 1 that is responsible for its interaction with a biological molecule. As used herein, “a precursor compound to a compound of formula 1” (also referred to as “precursor compound”) refers to a compound that is otherwise identical to a compound of formula 1 except that it does not contain a matrix and thus is not immobilized. One skilled in the art will be able to synthesize the precursor compounds or the core structure of a compound of formula 1 by methods known in the art, including the methods disclosed in U.S. patent application Ser. No. 10/438,152 and PCT/US/03/15193.

The present invention further comprises methods of identifying a biologically relevant target for a compound of formula 1. In one general aspect, the present invention provides a method of identifying a biological molecule that binds to a compound of formula 1, comprising the steps of: (1) contacting a test sample with the compound of formula 1 under a condition that allows a biological molecule within the test sample to bind to the compound, wherein said biological molecule is immobilized to a matrix via binding to the compound; (2) releasing the bound biological molecule from the matrix; and (3) characterizing the released biological molecule.

In another general aspect, the present invention provides a method of identifying a biological molecule that binds to a precursor compound to a compound of formula 1, comprising the steps of: (1) contacting a test sample with the precursor compound of formula 1 under a condition that allows a biological molecule within the test sample to bind to the precursor compound; (2) immobilizing the precursor compound to a matrix to form a compound of formula 1, wherein said biological molecule is immobilized to the matrix via binding to the compound; (3) releasing the bound biological molecule from the matrix; and (4) characterizing the released biological molecule.

As used herein, the term a “biological molecule” refers to any macromolecule that can be found in a living organism, such as a cell. Examples of “biological molecules” include, but are not limited to, lipids, carbohydrates, polypeptides, and polynucleotides.

Lipids are primarily hydrocarbon structures. They tend to be poorly soluble in water, and serve as a major component of the various membrane structures found in cells. Lipids also serve as a convenient, compact way to store chemical energy. Examples of lipids include, but are not limited to saturated or unsaturated fatty acids, steroids, prostalglandins, terpanes, waxes, triacylglycerol, and phospholipids.

Carbohydrates are primarily hydrocarbon structures as well, but they also contain many polar hydroxyl (—OH) groups and are therefore soluble in water. The most common carbohydrates are the simple six carbon (hexose) and five carbon (pentose) sugars. Carbohydrates also include polysaccharides, large carbohydrate molecules that consist of many small, ring-like sugar monomers attached to one another by glycosidic bonds in a linear or branched array. Examples of polysaccharides include, but are not limited to, glycogen, cellulose, or starch. In a cell, polysaccharides often form storage granules that may be readily broken down into their component sugars. Polysaccharides also serve as a major component of the cell wall.

Polypeptides are linear polymers of at least two amino acids held together by peptide linkage. Examples of polypeptides include short chains, which also commonly are referred to in the art as, e.g., peptides, oligopeptides and oligomers, and the longer chains, which generally are referred to in the art as proteins, of which there are many types. Examples of polypeptides also include the modified polypeptides. Modifications can occur anywhere in a polypeptide, including the peptide backbone, the amino acid side-chains and the amino or carboxyl termini. Several common modifications to a polypeptide, such as glycosylation, lipid attachment, sulfation, gamma-carboxylation of glutamic acid residues, hydroxylation and ADP-ribosylation, are described in many basic texts, including PROTEINS—STRUCTURE AND MOLECULAR PROPERTIES, 2nd Ed., T. E. Creighton, W. H. Freeman and Company, New York (1993). Many detailed reviews are also available on this subject, such as those provided by Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al. (1990), Meth. Enzymol. 182, 626-646; and Rattan et al., “Protein Synthesis: Posttranslational Modifications and Aging”, (1992) Ann. N.Y. Acad. Sci. 663, 48-62.

Proteins are the most complex macromolecules found in the cell. Many proteins are composed of two or more polypeptides held together by non-covalent forces. Some proteins have structural roles, for example to interact with lipids in membrane structures or to form part of the cytoskeleton that gives the cell its shape. Other proteins are the chief component of muscle or connective tissue. Yet, a major class of proteins known as enzymes function as catalysts that direct and accelerate biochemical reactions. Cells often contain thousands of different types of enzymes.

Polynucleotide is a chain structure containing at least 2 nucleotides joined together by phosphodiester bonds (5′-3′), and may comprise ribonucleotides and/or deoxyribonucleotides. Examples of polynucleotide include a deoxyribonucleic acid (DNA) molecule and a ribonucleic acid (RNA) molecule including, but not limited to, the messenger RNA (mRNA), the ribosomal RNA (rRNA), and the transfer RNA (tRNA). Examples of polynucleotides also include the short chains oligonucleotides, such as the small interference RNA (SiRNA). DNA contains the genetic information. Different types of RNA molecules serve different functions. For example, mRNA transmit the genetic information from DNA to a protein during protein synthesis, rRNA is found in ribosomes where protein synthesis takes place, and tRNA transports specific amino acids to the ribosomes, where they become linked into polypeptides. Oligonucleotides can also play an important role in the cell. For example, SiRNA binds to an RNA-induced silencing complex (RISC), can result in cleavage of the homologous mRNA sequence, thus specifically inhibits protein synthesis.

As used herein, “a test sample” refers to a sample containing or consisting of one or more biological molecules that are unknown or purported to bind to a compound of formula 1 or derivatives thereof. A test sample can be a test biological sample that has been the object of analysis, monitoring, or observation. Such a sample can be cells or biological fluids isolated from a subject. The subject can be a eukaryotic organism, such as an animal, a plant, a worm, or a yeast cell. Preferably, the subject is a mammal, such as a rat, a mouse, a monkey, or a human, who has been the object of treatment, observation or experiment. Examples of test biological samples include, for example, sputum, blood, blood cells (e.g., white blood cells), amniotic fluid, plasma, semen, bone marrow, tissue or fine-needle biopsy samples, urine, peritoneal fluid, pleural fluid, and cell cultures. Test biological samples can also include sections of tissues such as frozen sections taken for histological studies. In preferred embodiments, the test sample is a “clinical sample,” which is a sample derived from a human patient.

A test sample can be a library of synthetic or natural biological molecules. For example, it can be total cell or tissue homogenates or lysates, recombinant protein products resulting from expression of randomized oligonucleotides, a library of synthetic peptides, or a combination thereof. The test sample can be phage particles from a peptide or cDNA phage library. The test sample can be enriched for certain types of biological molecules, such as an immunoprecipitation of proteins. The test sample can also contain an isolated or purified biological molecule.

As used herein “binds” as used in “a biological molecule binds to a compound” refers to the close inter molecular interaction between the biological molecule and the compound such that they form a complex. Such an interaction can be covalent, i.e., a covalent bond is formed between the biological molecule and the compound. The interaction can also be non-covalent, i.e., the biological molecule and the compound can form a complex via one or more non-covalent interactions, such as van der Waals' forces, ionic interaction, or hydrogen bonding.

A biological molecule can bind to a core structure of a compound of formula 1 disregard whether the compound has been immobilized or not. For example, a biological molecule can bind to an immobilized compound of formula 1 as well as a precursor compound to a compound of formula 1 that is not immobilized.

In one embodiment, a biological molecule that binds to a compound of formula 1 can be isolated from a test sample by contacting the test sample with an immobilized compound of formula 1 to allow interaction of the immobilized compound with the biological molecule. The biological molecule becomes immobilized via binding to the compound. It can then be isolated from the test sample by methods consistent with the property of matrix used for immobilization.

In another embodiment, a biological molecule that binds to a compound of formula 1 can be isolated from a test sample by first contacting the test sample with a precursor compound to a compound of formula 1, allowing the biological molecule to bind to the precursor compound; then immobilizing to a matrix the precursor compound with or without the bound biological molecule. The biological molecule can then be isolated from the test sample by methods consistent with the property of matrix used for immobilization.

As used herein, the term “matrix” refers to a support that is an insoluble, functionalized, polymeric material to which a precursor compound to a compound of formula 1 can be attached to form a compound of formula 1, allowing the compound of formula 1 to be readily separated from excess reagents, soluble reaction by-products, or solvents. Materials suitable for matrix in the present invention include gels (e.g. dextrin or agarose), cellulose, glass, plastic material (e.g. polyethylene, polypropylene, polystyrene, polyamide, polyester, and the like), beads (e.g. magnetic, plastic, gel), and plates (metal, plastic, protein chips).

Commercially available matrices can be used in the present invention. For example, AminoLink® Coupling Gel and SulfoLink® Coupling Gel from Pierce Biotechnology, Inc. can be used. These are cross-linked beaded agarose supports that are reactive toward primary amines and sulfhydryl groups, respectively. UltraLink™ matrices from Pierce Biotechnology, Inc. can also be used. They are beaded biosupport containing bis-acrylamine/azlactone copolymer wherein a variety of functional groups may be present on the bead. Examples of such matrices include UltraLink™ DADPA (diaminodipropylamine); UltraLink™ Iodoacetyl support (a support activated with a terminal iodoacetyl group that reacted preferentially with sulfhydryl groups); and CarboLink™ coupling gel and UltraLink™ hydrazide gel (a beaded agarose derivatized to yield a terminal hydrazide group). Other types of matrices from Pierce Biotechnology, Inc. can also be used, such as PharmaLink™ gel, and Reacti-Gel® CDI Support (from Pierce Biotechnology, Inc.), a cross-linked beaded agarose solid support (referred to hereinafter as “GF2000”) that is reactive toward target molecules that contain an amino functional group. In addition, Protein Chip®Arrays from Ciphergen Biosystems, Inc. can also be used as matrix in the present invention. The protein chips include PS10 and PS20, referred to herein as “PS10 or PS20”. They are spot arrays, which have their surface activated by functional groups such as CDI (carbonyldiimidazole, PS10) or epoxy groups (PS20) for the covalent attachment of J¹ of a compound of formula 1 or precursor thereof.

Those skilled in the art will recognize that by selecting a matrix support with a desired functionality, a variety of chemical methods can be used for the attachment of the matrix to K via J¹ or —X-J³ to a precursor compound to a compound of formula 1 to form a compound of formula 1, wherein J¹ or J³ is as defined supra, and X is a specific binding pair. Immobilization of the precursor compound must occur at an attachment point that does not block the biologically important function of the core structure.

A preferred means of attachment of the matrix to form compounds of Formula 1 wherein L₄ is -M-K-J¹-Matrix or L₅ is -M¹-K-J¹-Matrix is through a covalent bond created between K and the matrix. The J¹ portion of the linker is the product of the reaction of a functionalized matrix with a functional group present on the terminus of K, wherein M, K, or J¹ is as defined supra.

Another preferred means of attachment of the matrix to form compounds of Formula 1 wherein L₄ is -M-K-J³-X-Matrix or L₅ is -M¹-K-J³-X-Matrix is through noncovalent interactions between the members of X, wherein M, M¹, K, or J³ is as defined supra, and X is a specific binding pair.

“A specific binding pair” is defined herein as a pair of molecules that have a high affinity for each other, resulting in a binding that is almost irreversible. For example, the specific binding pair (X) can consist of biotin (or a chemical derivative of biotin, such as iminobiotin) and its complementary protein such as avidin or streptavidin (Streptomyces avidinii). Avidin is an egg-white derived glycoprotein with an extraordinarily high affinity (affinity constant>10¹⁵ M⁻¹) for biotin. Streptavidin and NeutrAvidin™ have similar properties to avidin, but have a lower affinity for biotin. While streptavidin and NeutrAvidin™ are less stable than avidin, in most applications, streptavidin, NeutrAvidin™, and avidin are interchangeable.

The J³ portion of the linker is chemically derived as described above for J¹. An additional step involves the covalent attachment of biotin or the like to J³ using a biotinylating agent such as EZ-Link™ (from Pierce Biotechnology, Inc.). The complementary binding moity, avidin, streptavidin, or NeutrAvidin™, is purchased covalently bound to a matrix (e.g. ImmunoPure® Immobilized Avidin Gel, ImmunoPure® Immobilized Streptavidin Gel, or MagnaBind™ beads, from Pierce Biotechnology, Inc.)

Additional means for immobilizing a precursor compound include hydrophobic interactions, magnetic interactions, or polar interactions. For example, a magnetic bead (e.g. a bead capable of being magnetized such as a ferromagnetic bead) can be attracted to a magnetic support, and can be released from the support by the removal of the magnetic field. Alternatively, the bead can be provided with an ionic or hydrophobic moiety that can associate with, respectively, an ionic or hydrophobic moiety on a support.

After the biological molecule of interest is immobilized to a matrix via binding to a compound of formula 1, the methods of the present invention comprise a step of washing the matrix with a suitable buffer to remove non-specifically bound artifacts found in the test sample. The buffer suitable for removing non-specific binding but not the biological molecule of interest can be chosen by routine experimentation, such as by varying the pH or ionic strength of the buffer.

The biological molecules bound to an immobilized compound of formula 1 can be identified through various means of biological separation and detection known to a practitioner skilled in the art. For example, the bound biological molecule can be first released from the matrix, then isolated and characterized.

Various methods can be used to release a bound biological molecule from a matrix. In one embodiment, a bound biological molecule together with a precursor compound to a compound of formula 1 can be eluted from the matrix by the breakage of a disulfide linkage that affixes the precursor compound to the matrix (Example 1, infra). In another embodiment, a non-covalently bound biological molecule can be displaced from the immobilized compound of formula 1 by using excessive amount of a core structure compound of formula 1 (Example 2, infra). In yet another embodiment, a bound biological molecule together with a precursor compound to a compound of formula 1 can be displaced from the matrix by using excessive amount of biotin when the precursor compound of formula 1 is immobilized to the matrix by a specific binding pair of biotin and its complementary protein (Example 3, infra). In a particular enbodiment, when Ciphergen protein chip is used as the matrix, the biological molecule can be released from the chip by adding an energy-absorbing molecule (EAM) to the surface of the chip, followed by excitation with a nitrogen laser.

The released biological molecule can be further purified and characterized by methods known in the art for biological molecule separation and identification. For example, it can be first concentrated by appropriate concentration means, such as a membrane-based concentrator. The biological molecule can be subject to further analyses such as polyacrylamide gel electrophoresis (PAGE), see Laemmli, UK, Nature, 227, 680 (1970), Matrix Assisted Laser Desorption/Ionization-Time of Flight (MALDI-TOF), or chromatographic separations, see Ion exchange chromatography, Protein Purification, Principles, High resolution methods and applications, Ryden, L. (Eds) VCH, Publishers Inc. New York. (1989).

After isolation, the biological molecule of interest can be further identified. For example, a polypeptide biological molecule can be digested with trypsin or another proteolytic enzyme such as endoproteinase Lys-C, or S. aureus V8 protease whose fragmentation patterns are known. The digested protein is then analyzed by mass spectrometry (MS) to obtain the masses of the tryptic (or other enzyme) peptides. These masses can be compared to known peptide masses of digested proteins using a protein identification database, such as, for example, ProFound, Matrix Science Mascot, ProteinProspector and MOWSE. For any of these databases, the peptide masses are entered along with limitations of the search including, enzyme used for the digest, species, protein size and measurement error. Results will be given as probable matches for the identity of the protein based on digests of known proteins. Each identification will also be given a probability score so the user can determine if it is a valid identification or a random match. Z-scores of less than 2 are considered random while those greater that 2.6 carry a probability of greater than 95% of being accurate.

Since the identification obtained by the protein identification databases are based on a probability match of peptide fragments to fragment patterns of known proteins, a confirmation is preferred. To achieve this, several of the peptides generated by the enzyme digest can be further analyzed by tandem mass spectrometry. Using this technology, the peptide is collided with an inert gas to produce random amino acid fragments from which amino acid masses can be determined. These masses can be compared to databases to give an identification based on the amino acid sequence of each peptide. Matrix Science Mascot is an example of a database used for this analysis. If more than 2 of the analyzed peptides results in a sequence that matches the same protein then the identification is confirmed. Confirmation by this method requires more sophisticated equipment as well as additional time and money therefore it may not be performed in every instance.

Alternatively, an isolated polypeptide can be subject to N-terminal sequencing. The sequencing data can be compared to proteins in databases using standard methods such as Blast searches to identify the polypeptide. Similarly, sequence analyses can be performed when the biological molecule is a DNA. The identity of the DNA molecule can be searched by comparison of the sequencing data with a DNA database.

In a particular embodiment, phage display technology can be used for identifying polypeptides that binds to a compound of formula 1. Immobilized compound of formula 1 is used to adsorb phage particles from a peptide phage library. The process involves several steps that can be carried out by a practitioner skilled in the art. See, e.g., Rodi, et al., “Identifying of small molecule binding sites within proteins using phage display technology,” Combinatorial Chemistry and High Throughput Screening (2001), 4(7), 553-572.

The phage library is constructed such that individual phage particles display pieces of mammalian coding polypeptides via the insertion of mammalian cDNAs into the coding region of the phage surface protein gene. Phage particles that bind to an immobilized compound of formula 1 are isolated upon releasing from the matrix. They are subsequently used to infect E. coli cells to generate more phage particles, which are used for the next round assay. After several rounds of panning and phage amplification, phage particles that bind with high affinity to the compound of formula 1 are enriched. These phages are subject to DNA sequence analyses to determine the sequence of the mammalian cDNA that codes for a polypeptide that binds to the compound of formula 1. This sequence can be compared to DNA sequence databases to identify the compound interacting protein. For example, Rodi, D. J. et al., “Screening of a library of phage-displayed peptides identifies human bcl-2 as a taxol-binding protein,” JOURNAL OF MOLECULAR BIOLOGY 1999, 285(1), 197-203 reveals the use of biotinylated paclitaxel to discover novel binding proteins by phage display technology. Several phage display libraries and systems are available commercially. For example, a system such as that described above is commercially available from Novagen and is called the T7 select system (Catalog No. 70018). In addition, a variety of phage display libraries are also available from Novagen, for example a human colon tumor phage display T7 select library, Catalog No. 70645.

Once a potential biological target for the compound of interest is identified, the biologically relevant function of the compound can be examined by testing of the interaction of the compound with the biological target using a functional assay. Said assay preferably comprises comparing the biological activity of the biological molecule in the presence and absence of the compound of formula 1, or precursor or core compound thereof.

Selection of an appropriate assay will depend on the purported biological functions of the identified proteins. An example of such would be, but not limited to, a kinase assay if the purported function of the identified protein is to carry out a phosphorylation reaction of a substrate protein.

Another example would be, but not limited to, in vitro testing of the precursor compound in the presence of the purified protein in an ELISA assay, to examine if the precursor compound affects the binding or any other type of interaction of the identified protein with its purported interacting biomolecules. If the identified protein functions as a receptor, its binding to a known ligand can be examined in this fashion in the presence of the compound.

Alternatively, or in addition, the biological relevance of the discovered target of the precursor compound can further be assessed using knockout technologies (anti-sense RNA, siRNA, stem cell knockouts) to construct cellular systems devoid of the compound's target. In these systems the compound should not elicit the biological effect. Such a study provides further validation of the biological function of the precursor compound in a defined biological system. Such a study is also valuable if the identified protein is a novel biomolecule or a previously uncharacterized function. This type of study also further establishes if the identified protein is a potential novel molecular and therapeutic target.

The putative biological targets for a compound would need to be sufficient to explain the observed biological activities elicited by the compounds. For example, if a particular compound blocks tumor cells in a specific cell cycle stage (e.g. G2/M), the potential targets underlying this cell cycle block activity would need possess this function of inducing G2/M cell cycle arrest.

### 2.B EXAMPLES

In the subsequent examples, compounds of formula 1, the precursor, or core structure compounds thereof were based on the compounds disclosed in U.S. patent application Ser. No. 10/438,152 and PCT/US/03/15193 and synthesized as disclosed therein. The compounds were known to be useful in the treatment of cell proliferative disorders, disorders related to PDGF receptor such as tumors, restenosis, rheumatoid arthritis, diabetic retinopathy, and the like.

Compounds of formula 1 and the precursor compounds thereof can be used as investigative research tools for the identification of biomolecular/cellular targets other than the target, PDGF-RTK. Methods of the present invention allowed for selectivity profiling against additional protein kinases, enzymes, cellular proteins, or other relevant biomolecules, for toxicity prediction, compound optimization and exploration in biological systems for additional therapeutically relevant biomolecular targets. In another embodiment, methods of the present invention can be used for identifying additional molecular target(s) that underlie the anti-proliferative activity discovered for the precursor compounds disclosed in U.S. patent application Ser. No. 10/438,152 and PCT/US/03/15193. In yet another embodiment, the present invention allowed for identification of secondary actions of the precursor compounds and the identification of potential side effects and/or additional therapeutic benefits and indications for the precursor compounds.

The following examples illustrate methods of identifying a protein that binds to a core structure of formula 1. The process described thereafter involves several steps that can be carried out by a practitioner skilled in the art. A person of ordinary skill in the art is also able to identify biological molecules other than proteins that bind to a compound of formula 1, using similar methods with modifications suitable for identifying the specific type of target molecule, such as nucleic acids, lipid, or carbohydrates. The following examples are given for the purpose of illustrating various aspects of the present invention and are not meant to limit the present invention in any fashion.

### Example 1

### Identification of a Protein that Covalently or Non-Covalently Interacts with a Compound of Formula 1 Containing a Biotin Substituent

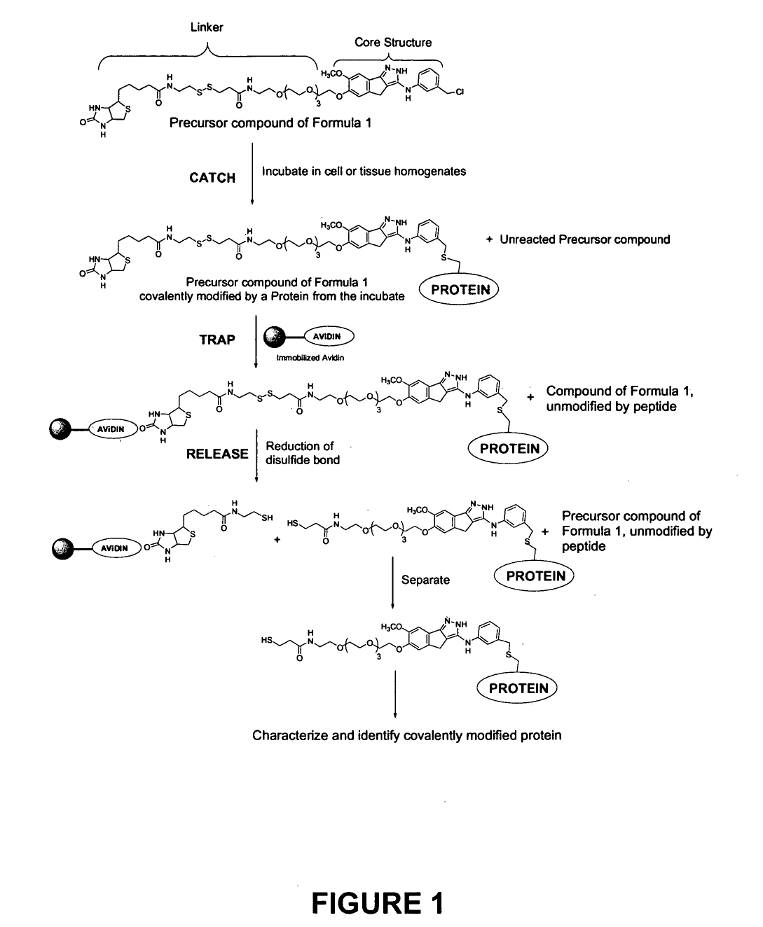

FIG. 1 depicts a method of the present invention for the discovery of proteins that covalently bind to precursor compounds of Formula 1 or that form high affinity non-covalent interactions with precursor compounds of Formula 1. In particular, the method depicted in FIG. 1 initially involves a particular precursor compound of Formula 1 containing a biotin substituent covalently attached to a linker via a disulfide bond. The exemplified method is equally applicable to any precursor compound of Formula 1 containing a biotin substituent incorporated via a disulfide linkage.

During the “CATCH” step, the precursor compound of Formula I is incubated with cell or tissue homogenates in an incubation mixture for a time ranging from 10 min to 24 h, at temperatures ranging from 0° C. to 37° C. In addition to the precursor compound and cell or tissue homogenates, the incubation mixture further comprises optimized media containing protease inhibitors and standard buffers, such as PBS (phosphate buffered saline) and RIPA (radioimmunoprecipitation buffer).

Upon completion of the “CATCH’ step, some or all of the precursor compound of Formula 1 may have formed a covalent or non-covalent bond with one or more types of proteins in the cell or tissue homogenates via a reactive amino acid side chain, such as a thiol group from a cysteine (as illustrated in the example in FIG. 1), an amino group from a lysine or a N-terminal amino acid, a guanidine from an arginine, a hydroxyl group of a serine or threonine, or a carboxylate group from either aspartic acid, glutamic acid or the carboxylate of a C-terminal amino acid or by means of a substantially irreversible, high affinity non-covalent interaction.

During the “TRAP” step, the incubation mixture is exposed to a biotin complementary protein that is immobilized to a matrix. Any type of immobilized biotin complementary proteins, such as immobilized Avidin or streptavidin can be used during the TRAP step. See for example, Avidin-biotin immobilization systems. Wilchek, Meir; Bayer, Edward A.; Editor(s): Cass, Tony; Ligler, Frances S. Immobilized Biomolecules in Analysis (1998), 15-34. Publisher: Oxford University Press, Oxford, UK). The immobilized biotin complementary protein forms a specific binding pair with the biotin substituent on the precursor compound, which in turn immobilizes the precursor compound to the matrix to form the compound of formula 1. Because some or all of the immobilized precursor compound of formula 1 has formed a covalent or non-covalent bond with one or more types of proteins in the cell or tissue homogenates, the compound-interacting protein(s) is also immobilized, thus separated from other components in the cell or tissue homogenates. Non-specific binding artifacts found in the incubation mixture can be removed by washing the matrix with suitable buffer.

During the “RELEASE” step, the disulfide bond that incorporates the biotin to the compound of formula (I) is cleaved to form two thiol-terminated species. One of the thiol-terminated species is the matrix bound specific binding pair consisting of biotin and the biotin complementary protein, such as the immobilized avidin/biotin specific binding pair. The other thiol-terminated species is a precursor compound to a compound of formula 1 that is either free from any bounded protein or has been covalently or non-covalently modified with a protein or several proteins found in the incubate. The precursor compound no long has the biotin substituent as compared to that in the “CATCH” step.

Cleavage of the disulfide bond can be effected by any of the mild chemical methods known in the art for reduction of a disulfide bond to the constituent thiol groups, for example, by the use of compound such as sodium borohydride, sodium triacetoxy borohydride, sodium cyanoborohydride, potassium triisopropoxyborohydride or dithiothreitol in water, aqueous alcohol or aqueous THF or aqueous dimethyl sulfoxide or aqueous dimethyl formaimde or aqueous dimethyl acetamide solution at temperatures ranging from 0° C. to 37° C. with exposure times ranging from 1 min to 24 h or more. See Brown, H. C. et al., J. Org. Chem., 1984, 885.

After cleavage of the disulfide linkage, the two thiol-terminated species are separated by methods consistent with the immobilization matrix. Such methods include, but are not limited to, physical removal of avidin that has been immobilized on a chip, elution from a chromatography column for avidin that has been immobilized on a chromatographic support such as agarose, sephadex or sepharose, or by the use of magnets for avidin that has been immobilized on a magnetic nanoparticle. See Applications of magnetic nanoparticles in biomedicine. Pankhurst, Q. A.; Connolly, J.; Jones, S. K.; Dobson, J; Journal of Physics D: Applied Physics (2003), 36(13), R167-R181.

The precursor compound of Formula 1 that has been covalently or non-covalently modified with a protein or several proteins found in the incubation mixture can be further separated from the precursor compound that is free from any bounded protein by any of the typical separation methods, which utilize either protein charge or protein molecular weight for separation. Such methods include, but are not limited to size-exclusion chromatography, gel electrophoresis, or Western blot or high performance liquid chromatography. Using similar methods, the protein or proteins bound to the precursor compound of Formula 1 can be further isolated to homogeneity. The homogeneous protein can then be characterized and identified by any of the typical methods know to a practitioner skilled in the art of protein identification such as, but not limited to, Matrix Assisted Laser Desorption/Ionization-Time Of Flight (MALDI-TOF) or Surface Enhanced Laser Desorption/Ionization-Time of Flight (SELDI-TOF) Mass Spectroscopy, micro-sequencing, capillary or gel electrophoresis, Western blot, or N-terminal sequencing.

### Example 2

### Identification of a Protein that Forms a Reversible, Non-Covalent Complex with a Compound of Formula 1

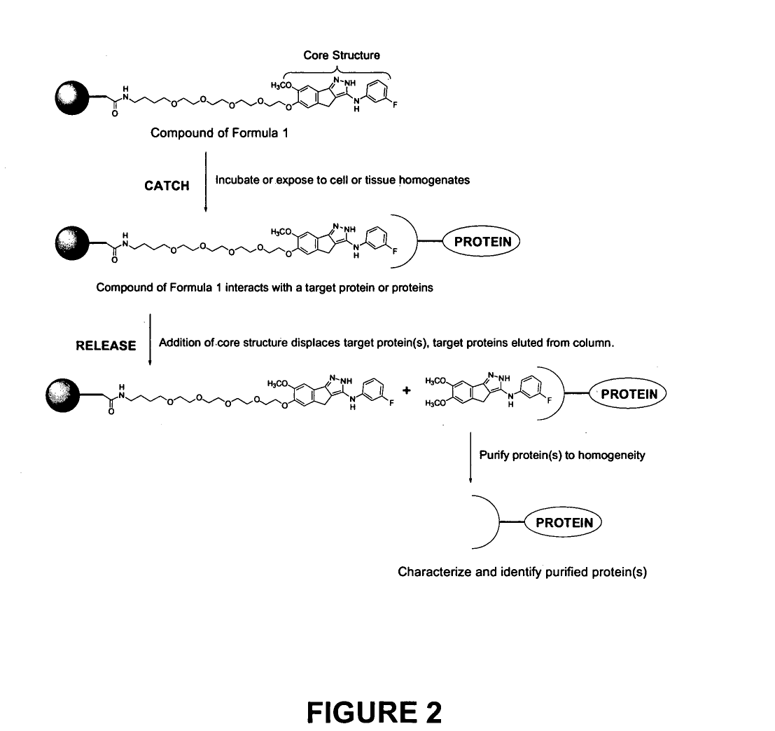

FIG. 2 depicts a method of the present invention for the discovery of a protein that forms a reversible, non-covalent complex with a compound of Formula 1. In particular, the method initially involves a particular compound of formula 1 that is affixed to a matrix. The method of the present invention exemplified herein, however, is equally applicable to any compound of Formula 1.

During the “CATCH” step, the compound of Formula I is incubated with cell or tissue homogenates in an incubation mixture for a time ranging from 10 min to 24 h, at temperatures ranging from 0° C. to 37° C. In addition to the compound and cell or tissue homogenates, the incubation mixture further comprises optimized media containing protease inhibitors and standard buffers, such as PBS (phosphate buffered saline) and RIPA (radioimmunoprecipitation buffer).

Upon completion of the ‘CATCH’ step, some or all of the compound of Formula 1 may have formed a reversible, non-covalent complex with one or more types of proteins in the cell or tissue homogenates via a reversible non-covalent interaction with the core structure portion of Formula 1. The interaction is of sufficient affinity, which allows for the immobilization of the one or more types of proteins to the matrix of the compound of formula 1, thus the separation of this protein(s) from other components of the cell or tissue homogenates. Non-specific binding artifacts found in the incubation mixture can be removed by washing the matrix with suitable buffer.

During the “RELEASE” step, excess amounts of simple analogs of the core structure portion of formula 1 are added to displace the protein(s) that bind to the compound of formula 1 via a reversible non-covalent interaction. The displaced protein(s) is then separated from the immobilized avidin/biotin specific binding pair by methods consistent with the immobilization matrix. Such methods include, but are not limited to, physical removal of avidin that has been immobilized on a chip, elution from a chromatography column for avidin that has been immobilized on a chromatographic support such as agarose, sephadex or sepharose, or by the use of magnets for avidin that has been immobilized on a magnetic nanoparticle. See Applications of magnetic nanoparticles in biomedicine. Pankhurst, Q. A.; Connolly, J.; Jones, S. K.; Dobson, J; Journal of Physics D: Applied Physics (2003), 36(13), R167-R181.

The protein or proteins that bind to the compound of formula 1 via a reversible non-covalent interaction can then be further purified to homogeneity by any of several methods that utilize either protein charge or protein molecular weight. Such methods include, but are not limited to, size-exclusion chromatography, gel electrophoresis, Western blot or high performance liquid chromatography. The homogeneous protein can then be characterized and identified by any of the typical methods know to a practitioner skilled in the art of protein identification such as, but not limited to, Matrix Assisted Laser Desorption/Ionization-Time Of Flight (MALDI-TOF) or Surface Enhanced Laser Desorption/Ionization-Time of Flight (SELDI-TOF) Mass Spectroscopy, micro-sequencing, capillary or gel electrophoresis, Western blot, or N-terminal sequencing.

### Example 3

### Identification of a Protein that Forms a Reversible, Non-Covalent Complex with a Compound of Formula 1 Containing a Biotin Substituent

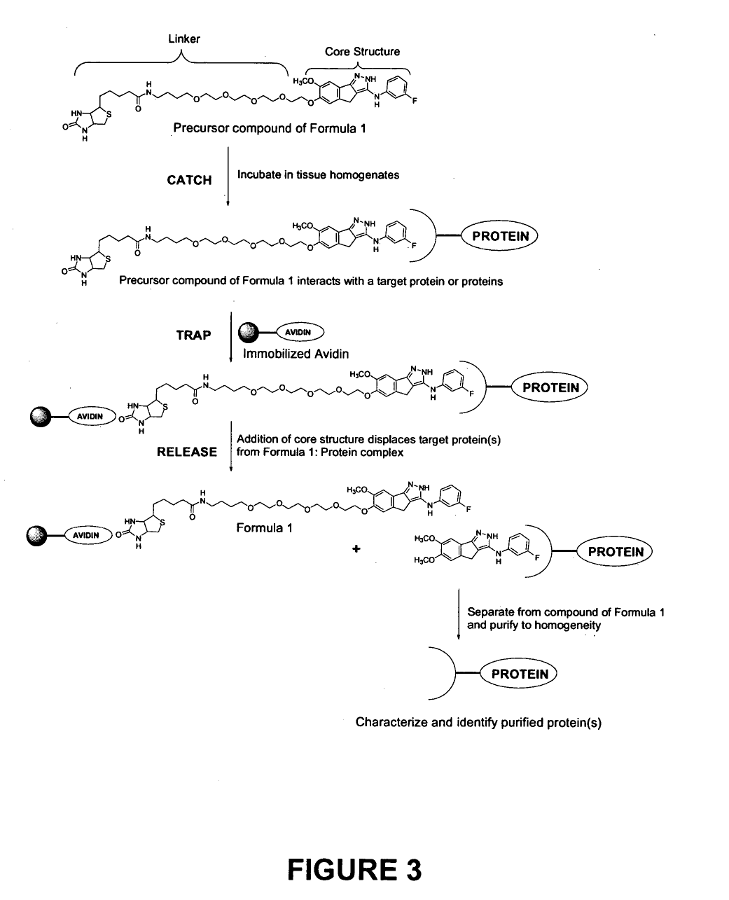

FIG. 3 depicts a method of the present invention for the discovery of a protein that forms a reversible, non-covalent complex with a compound of Formula 1. In particular, the method initially involves a particular precursor compound of formula 1 containing a biotin substituent that is linked to the compound without via a disulfide bond. The exemplified method is equally applicable to any precursor compound of Formula 1 containing a biotin substituent.

During the “CATCH” step, the precursor compound of Formula I is incubated with cell or tissue homogenates in an incubation mixture for a time ranging from 10 min to 24 h, at temperatures ranging from 0° C. to 37° C. In addition to the precursor compound and cell or tissue homogenates, the incubation mixture further comprises optimized media containing protease inhibitors and standard buffers, such as PBS (phosphate buffered saline) and RIPA (radioimmunoprecipitation buffer).

Upon completion of the ‘CATCH’ step, some or all of the precursor compound of Formula 1 may have formed a reversible, non-covalent complex with one or more types of proteins in the cell or tissue homogenates via a reversible non-covalent interaction with the core structure portion of Formula 1. The interaction is of sufficient affinity.

During the “TRAP” step, the incubation mixture is exposed to a biotin complementary protein that is immobilized to a matrix. Any type of immobilized biotin complementary proteins, such as immobilized Avidin or streptavidin can be used during the TRAP step. See for example, Avidin-biotin immobilization systems. Wilchek, Meir; Bayer, Edward A.; Editor(s): Cass, Tony; Ligler, Frances S. Immobilized Biomolecules in Analysis (1998), 15-34. Publisher: Oxford University Press, Oxford, UK). The immobilized biotin complementary protein forms a specific binding pair with the biotin substituent on the precursor compound, which in turn immobilizes the precursor compound to the matrix to form the compound of formula 1. Because some or all of the immobilized precursor compound of formula 1 has formed a reversible, non-covalent interaction with one or more types of proteins in the cell or tissue homogenates, the compound-interacting protein(s) is also immobilized, thus separated from other components in the cell or tissue homogenates. Non-specific binding artifacts found in the incubation mixture can be removed by washing the matrix with suitable buffer.

During the “RELEASE” step, excess amounts of simple analogs of the core structure portion of formula 1 are added to displace the protein(s) that bind to the compound of formula 1 via a reversible non-covalent interaction. Alternatively, excess amount of free biotin can also be used to displace the reversible non-covalently bound protein(s). The displaced protein(s) is then separated from the immobilized avidin/biotin specific binding pair by methods consistent with the immobilization matrix. Such methods include, but are not limited to, physical removal of avidin that has been immobilized on a chip, elution from a chromatography column for avidin that has been immobilized on a chromatographic support such as agarose, sephadex or sepharose, or by the use of magnets for avidin that has been immobilized on a magnetic nanoparticle. See Applications of magnetic nanoparticles in biomedicine. Pankhurst, Q. A.; Connolly, J.; Jones, S. K.; Dobson, J; Journal of Physics D: Applied Physics (2003), 36(13), R167-R181.

The protein or proteins that bind to the compound of formula 1 via a reversible non-covalent interaction can then be further purified to homogeneity by any of several methods that utilize either protein charge or protein molecular weight. Such methods include, but are not limited to, size-exclusion chromatography, gel electrophoresis, Western blot or high performance liquid chromatography. The homogeneous protein can then be characterized and identified by any of the typical methods know to a practitioner skilled in the art of protein identification such as, but not limited to, Matrix Assisted Laser Desorption/Ionization-Time Of Flight (MALDI-TOF) or Surface Enhanced Laser Desorption/Ionization-Time of Flight (SELDI-TOF) Mass Spectroscopy, micro-sequencing, capillary or gel electrophoresis, Western blot, or N-terminal sequencing.

### Example 4

### Identification of a Protein that Forms a Reversible, Non-Covalent Complex with a Compound of Formula 1 Bound to a Chip System

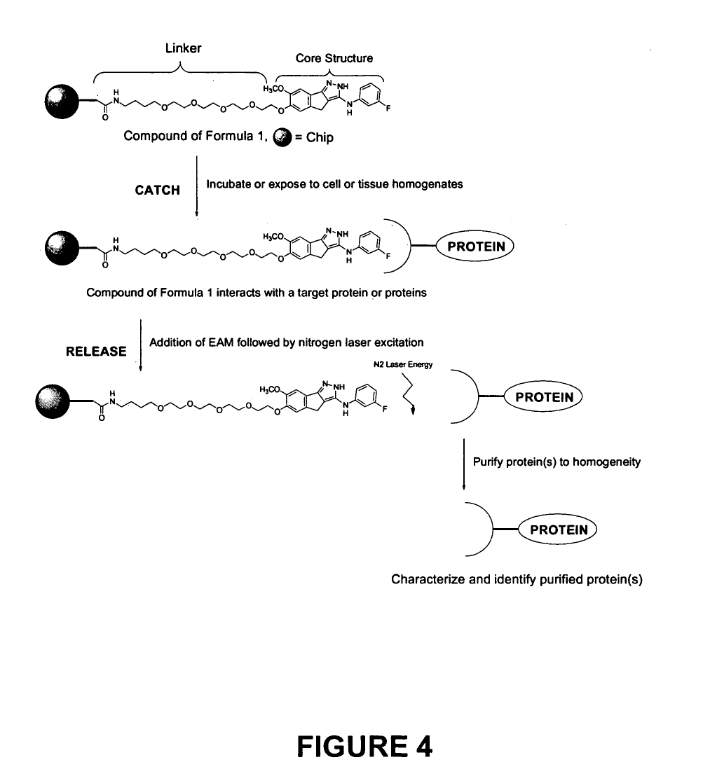

FIG. 4 depicts a method of the present invention for the discovery of proteins that interact with a compound of Formula 1 bound to a Ciphergen PS10 or PS20 ProteinChip. The method of the present invention exemplified herein, however, is equally applicable to any compound of Formula 1 immobilized to a protein chip system.

The Ciphergen ProteinChip system is a tool that allows for the study of proteins through Retentate Chromatography™ (Ciphergen Biosystems, Inc.). In this system, proteins bound to the chip surface are released in ionized form by a nitrogen laser, and their molecular weights are determined by time of flight (TOF) mass spectroscopy. The technology is known as Surface Enhanced Laser Desorption/Ionization-Time of Flight (SELDI-TOF) Mass Spectroscopy. Advantages of this method over other forms of protein chromatography include rapid optimization of chromatographic conditions and minimization of protein loss prior to detection. See: Chapman K., “The ProteinChip Biomarker System from Ciphergen Biosystems: a novel proteomics platform for rapid biomarker discovery and validation,” Biochem Soc Trans. 2002 April; 30(2): 82-7.

A number of chemistry-based surfaces are commercially available for the binding of proteins based on characteristics such as charge or hydrophobicity (Ciphergen, Dumbarton, Calif.). Also available are chemically reactive surfaces intended for the binding of biomolecules as bait for protein-protein interactions or nucleic acid-protein interactions, for example, the PS10 and PS20 ProteinChips from Ciphergen coated with carbonyl diimidazole and epoxy groups respectively. These coating groups allow the attachment of proteins or nucleic acids though covalent bonds directly to the chip. See: Hinshelwood, et al., “Identification of the C3b Binding Site in a Recombinant VWF-A Domain of Complement Factor B by Surface-enhanced Laser Desorption-Ionisation Affinity Mass Spectrometry and Homology Modeling: Implications for the Activity of Factor B,” Journal of Molecular Biology, 294(2) 1999, 587-599; Forde, C. E et al., “Characterization of transcription factors by mass spectrometry and the role of SELDI-MS,” Mass Spectrom. Rev. (2002 November-December), 21(6), 419-39.

The proteins or nucleic acids bound to the chip surface can serve as bait to capture proteins that specifically interact with the bound biomolecules. The present invention makes use of these reactive surface chemistries intended for the immobilization of large biomolecules to alter the PS10 and PS20 chip surfaces in a unique way. The chips are reacted with a precursor compound to a compound of formula 1 to form a compound of Formula 1. The linker of the compound of Formula 1 is preferably fashioned to allow movement of the compound away from the chip surface in a three-dimensional space. This will theoretically allow more binding as the binding protein can access the core structure more readily (See Improving protein-ligand interactions. Wandless, Thomas J. Department of Chemistry, Stanford University, Stanford, Calif., USA. Book of Abstracts, 219th ACS National Meeting, San Francisco, Calif., Mar. 26-30, 2000 (2000)). In most cases, a short tether of a linker consisting, for example four carbons, will not allow adequate space for protein binding. Preferred tethers comprise at least an eight-atom linker or the equivalent length.

Prior to the ‘CATCH’ step of FIG. 4, a precursor compound to a compound of formula 1 was attached to a PS10 or PS20 ProteinChip to form a compound a formula 1 wherein the matrix is a PS10 or PS20 ProteinChip. In general, the chips were set in a 8×12 platform reaction block and incubated with the compounds in solution such as DMF, THF, dichloromethane etc. at temperature ranging from 0 to 150° C. for several hours. The chip surface was then washed and equilibrated with the binding buffer (PBS, 0.1% Triton-X-100) three times each for about 10 minutes.

During the “CATCH” step, protein lysates from LoVo cells (ATCC CCL-229) prepared using M-Per®, Mamalian Protein Extraction Reagent (Pierce, Rockford, Ill.) were fractionated into 6 fractions based on isoelectric point (pl). A strong anion exchange column was used for the fractionation. After the column was loaded with lysates, it was washed with pH 9.0 (50 mM Tris), pH 7.0 (0.1 M NaPO₄), pH 5.0 (0.1M NaAcetate), pH 4.0 (0.1M NaAcetate), and pH 3.0 buffers (0.05 M Na Citrate) each containing 0.1 nOctyl beta-D-glucopyranoside (OGP), followed by an organic wash (33.3% isopropanol/16.7% acetonitrile/0.1% trifluoroacetic acid). The fractions were then diluted to 0.5 mg/ml in 100 μl of binding buffer and incubated on the compound-linked chip surface for 4 h at 37° C. Other possible sources of proteins that can be incubated with the compound are serum, plasma, urine, cell or tissue homogenates with an incubation time ranging from 10 min to 24 h, at temperatures ranging from 0° to 37° C. in optimized media containing protease inhibitors and a common buffer such as PBS or RIPA.

Upon completion of the ‘CATCH’ step, some or all of the compound of Formula 1 may have formed a reversible, non-covalent complex with one or more types of proteins in the cell or tissue homogenates via a reversible non-covalent interaction with the core structure portion of Formula 1. The interaction is of sufficient affinity, which allows for the immobilization of the one or more types of proteins to the protein chip, thus the separation of this protein(s) from other components of the cell or tissue homogenates. Non-specific binding artifacts found in the incubation mixture can be removed by washing the chip with suitable buffer. In this case, the chips were washed with the binding buffer three times each for about 15 minutes at room temperature to remove any non-specific binding artifacts.

During the “RELEASE” step, the protein(s) were released from the ProteinChip surface using the surface enhanced laser/desorption ionization time of flight (SELDI-TOF) technology. The Ciphergen ProteinChip Reader (PBS IIc) was used, which consists of adding an energy-absorbing molecule (EAM) to the chip, followed by excitation with a nitrogen laser.

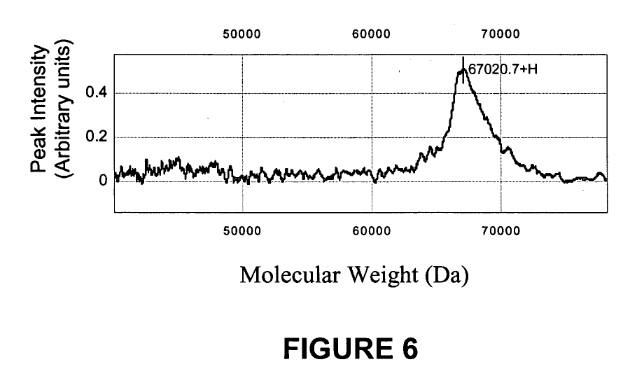

To confirm specific binding to the core structure, the peaks from the spectra generated from the chips coated with Formula 1 were compared with spectra from chips coated with the control compound, i.e., the aliphatic linker. An example of the unique peak from a pH 9.0 fraction is shown in FIG. 6, when Compound 6 of the present invention was used to capture the associated proteins.

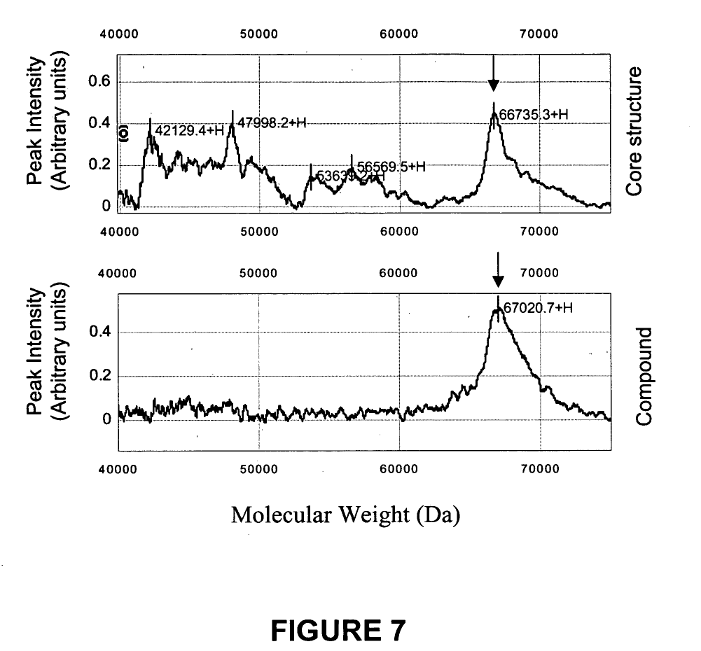

Results from the chip studies were compared to those from studies using an ATP affinity column. The LoVo cancer cell lysate was first passed through an ATP affinity column to allow ATP-binding proteins in the lysate to be bound to the column. A solution of an un-tethered analog corresponding to the core structure of compound of formula 1 was added to the ATP column to elute the protein(s) that also binds to the compound of formula 1, wherein the interaction between ATP and the eluted protein(s) is reversible and non-covalent. As shown in FIG. 7 (top graph), several protein peaks were eluted from the ATP column with a solution of an un-tethered analog corresponding to the core structure of compound 6 of the present invention. Among them, the major protein peak is identical to the unique protein peak captured using a compound 6 affixed to a PS10 protein chip (bottom graph of FIG. 7). The results of the ATP column studies provided an independent validation of the method of the invention. The data demonstrated that compounds of Formula 1, wherein the matrix is a chip surface, can be used to capture proteins that form a substantial intermolecular binding interaction with the core structure of formula 1.

The data also revealed that the position of the tether is critical in determining a protein's capability to specifically bind to the core structure. Compounds of Formula 1 with linkers at different positions on the core structure were used in the chip study. Each of these compounds yielded varying results depending on the position of the tether. Therefore, to completely profile the potential of a core structure to interact with protein targets known and yet to be discovered, it is preferable to repeat the method of the present invention with several analogs of the core structure in which the tether is substituted at different locations on the core structure.

The isolated target protein or several target proteins may then be purified to homogeneity by any of several methods that utilize either protein charge or protein molecular weight such as, but not limited to, size-exclusion chromatography, gel electrophoresis, Western blot or high performance liquid chromatography. The homogeneous protein can then be characterized and identified by any of the typical methods known to a practitioner skilled in the art of protein identification such as, but not limited to, MALDI-TOF or further SELDI-TOF Mass Spectroscopy, micro-sequencing, capillary or gel electrophoresis, enzymatic activity, Western blot, or N-terminal sequencing.

### Example 5

### Identification of a Protein that Forms a Reversible, Non-Covalent Complex with a Compound of Formula 1 Containing a Biotin Substituent Using a Protein Chip System

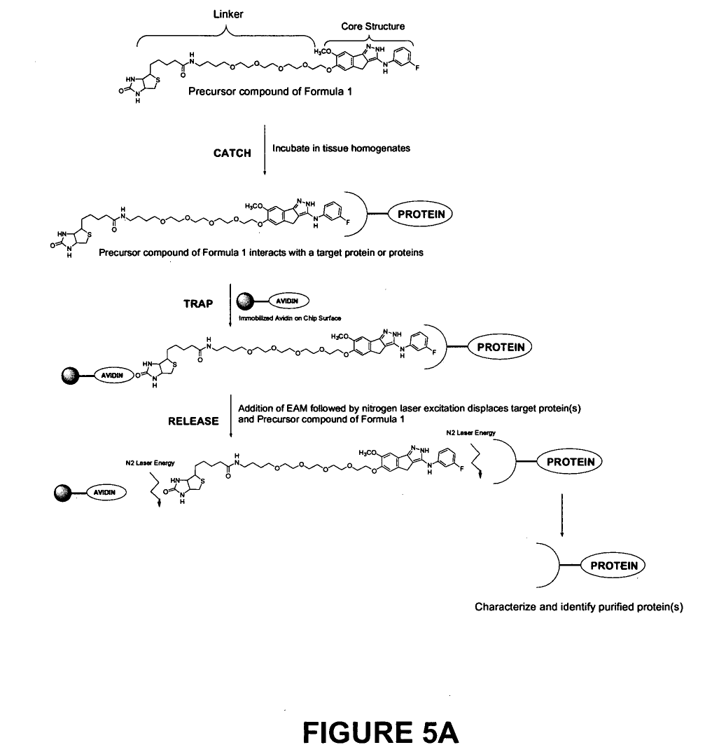

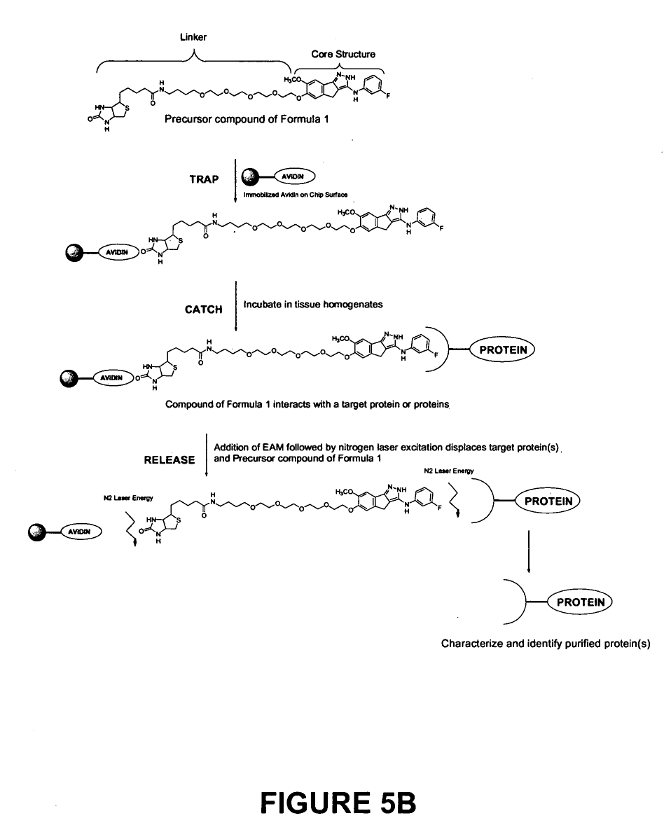

FIGS. 5A and 5B depict a method for the discovery of proteins that interact with precursor compounds of Formula 1 that contain a biotin substituent using a protein chip system. The method of the present invention as exemplified herein, however, is equally applicable to any derivative of Formula 1 containing a biotin substituent using a protein chip system.

A compound containing a biotin substituent can be indirectly attached to a chip surface using covalently bound streptavidin (SA) on the chip surface (Bane T K, LeBlanc J F, Lee T D, Riggs A D. DNA affinity capture and protein profiling by SELDI-TOF mass spectrometry: effect of DNA methylation. Nucleic Acids Res. 2002 Jul. 15;30(14):e69). Therefore the precursor compound of formula 1 containing a biotin substituent can be immobilized onto a Ciphergen PS10 or PS20 ProteinChips coated with a biotin complementary protein, such as streptavidin or Avidin. This immobilization or “TRAP” step can be performed either before (FIG. 5B) or after (FIG. 5A) the “CATCH” step, where the precursor compound is incubated with a biological sample, such as cell or tissue homogenates.

In FIG. 5A, during the ‘CATCH’ step, a precursor compound of Formula I comprising a biotin substituent is incubated with serum, plasma, urine, cell lysates or tissue homogenates for a time ranging from 10 min to 24 h, at temperatures ranging from 0° to 37° C. in optimized media containing protease inhibitors and buffers such as PBS, RIPA and others. Upon completion of the ‘CATCH’ step, some or all of the core structure of the precursor compound of Formula 1 may have formed a reversible, non-covalent complex with a protein or several proteins in the tissue homogenate of sufficient affinity to allow for isolation. The protein(s) that binds to the precursor compound of Formula 1 is then isolated from the incubation mixture in the ‘TRAP’ step, during which the incubation mixture is added to a protein chip coated with high affinity cognate proteins for biotin, such as avidin or streptavidin. Suitable washing step is performed to remove non-specific binding moieties found in the incubation mixture. During the “RELEASE” step, the bound protein(s) and precursor compound are released from the ProteinChip surface into a time of flight mass spectrometer by addition of an energy-absorbing molecule (EAM) followed by excitation with a nitrogen laser.

FIG. 5B outlines a variation to the method of FIG. 5A. It employs the TRAP step prior to the CATCH step and therefore forming an immobilized compound of Formula 1 on the chip prior to incubating the compound with a biological sample.

The isolated target protein or several target proteins found by either method may then be purified to homogeneity by any of several methods that utilize either protein charge or protein molecular weight such as, but not limited to, size-exclusion chromatography, gel electrophoresis, Western blot or high performance liquid chromatography. The homogeneous protein can then be characterized and identified by any of the typical methods known to a practitioner skilled in the art of protein identification such as, but not limited to, MALDI-TOF or further SELDI-TOF Mass Spectroscopy, micro-sequencing, capillary or gel electrophoresis, or Western blot, or N-terminal sequencing.

### Example 6

### Compound 124 of the Invention Forms a Reversible, Non-Covalent Complex with Polymerizing Tubulin

Using the method described in FIG. 5B, a protein shown to be associated with Compound 124 was identified as tubulin. To confirm the result, purified tubulin was used during the “CATCH” step in a method similar to that of FIG. 5B to demonstrate that tubulin indeed binds to Compound 124.

A Ciphergen PS20 chip was coated with 2 μL of 0.5 mg/ml streptavidin (SA). The SA-coated chip surface was then exposed to 10 μM precursor compound of compound 124 to form Compound 124 of Formula 1, which has the Ciphergen PS20 chip as the matrix. The SA-coated chip surface was exposed to 10 μM biotin alone to form a control compound, which has biotin bound to the PS20 chip but does not have the core structure of compound 124. Excess precursor compound or biotin was removed by washing the chip surface three times each for 10 min with PBS.

During the ‘CATCH’ step, Compound 124 was incubated for 1 h with 10 mg/mL purified tubulin in the presence or absence of 1 mM GTP, which allows for polymerization. In a third condition of the ‘CATCH’ step, the same amount of tubulin was first polymerized for 30 min in the presence of 1 mM GTP in a tube and then incubated on the chip surface of Compound 124 for 1 h. The chips were washed 3 times with PBS, 0.1% triton to remove unbound protein.

During the “RELEASE” step, the bound protein(s) and precursor compound are released from the ProteinChip surface into a time of flight mass spectrometer by addition of an energy-absorbing molecule (EAM) followed by excitation with a nitrogen laser. Specific binding to Compound 124 was determined by comparing peaks from time of flight spectra generated from Compound 124 to that generated from biotin-coated chips.

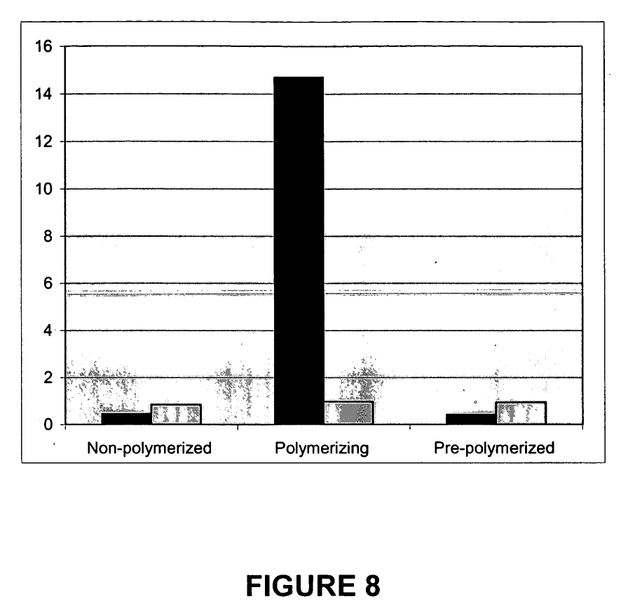

As shown in FIG. 8 polymerizing tubulin binds to compound 124. Greater than 10 fold polymerizing tubulin bound to compound 124 as compared to that bound to the control compound. In addition, FIG. 8 illustrates that significantly more tubulin bound to Compound 124 when the tubulin was in the polymerizing state, i.e., when the tubulin was incubated with Compound 124 in the presence of GTP. No significant binding to the compound was detected with non-polymerized tubulin (no GTP during the “CATCH” step) or pre-polymerized tubulin (polymerized prior to the “CATCH” step).

The discovery that a compound of the series of N-substituted tricyclic 3-aminopyrazoles compounds binds polymerizing tubulin is an interesting one. Agents that bind to tubulin have been shown to exert antimitotic effects by disrupting the function of the mitotic spindle, an essential component of the cytoskeleton that is involved in cell motility and transport. Such agents have been proven effective in the treatment of various cancers.

The two main families of antitubulin drugs are the taxanes and the vinca alkaloids. The taxanes include paclitaxel and docetaxel. Paclitaxel is used mainly in the treatment of ovarian, lung and breast cancer, and is being investigated for use as a single agent for the treatment of small cell lung cancer (SCLC), non-small cell lung cancer (NSCLC), advanced head and neck cancers, and adenocarcinomas of the upper gastrointestinal tract. Docetaxel is used mainly to treat locally advanced or metastatic breast and lung cancer after chemotherapy has failed. The vinca alkaloids include vincristine, vinblastine, and vinorelbine. Vincristine and vinblastine are most commonly used in combination therapy regimens. For example, Vinblastine has been used in the treatment of Hodgkins disease, some lymphomas, and neuroblastoma.

Two newer families of tubulin-binding drugs are the epothilones and the dolastintins. The epothilones (A and B) are naturally occurring macrocyclic lactones isolated from the soil bacterium Polyangium cellulosum. They share a similar mode of action to the taxanes and are being investigated in clinical trials. The dolastatins are derived from the sea hare (Dolabella auricularia). They share a similar mode of action to that of the vincas, and are currently being investigated.

The discovery that a compound of the series of N-substituted tricyclic 3-aminopyrazoles compounds binds to polymerizing tubulin provides a method of identifying a compound that binds to tubulin, comprising the steps of: (a) synthesizing a compound that mimics the core structure of a compound of formula 1; and b) determining the ability of the compound that mimics the core structure of a compound of formula 1 to bind to polymerizing tubulin.

The discovery that a compound of the series of N-substituted tricyclic 3-aminopyrazoles compounds binds to polymerizing tubulin further provides a method of regulating an activity of polymerizing tubulin comprising a step of contacting the polymerizing tubulin with a compound of formula 1, a precursor compound to a compound of formula 1, or a core structure of a compound of formula 1.

The discovery further provides a method of disrupting the function of the mitotic spindle in a cell comprising the step of contacting the cell with a compound of formula 1, a precursor compound to a compound of formula 1, or a core structure of a compound of formula 1.

1. A compound of Formula (I): 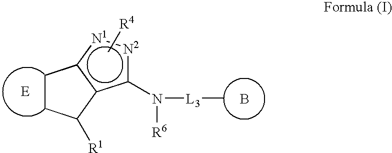
or an optical isomer, enantiomer, diastereomer, racemate, or pharmaceutically acceptable salt thereof, wherein: 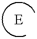
is selected from the group consisting of Formulae A-1, A-2 and A-3: 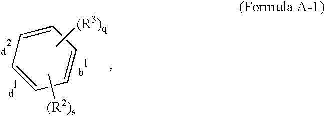
wherein Formula A-1 is attached on the b¹ side of Formula A-1 to the R¹-substituted ring of formula (I) and optionally substituted with one substituent selected from the group consisting of Formulae A-1-a, A-1-b and A-1-c: 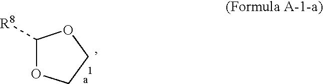
wherein Formula A-1-a is attached on the a¹ side to adjacent carbons on the d¹ or d² side of Formula A-1; 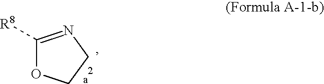
wherein Formula A-1-b is attached on the a² side to adjacent carbons on the d¹ or d² side of Formula A-1; and 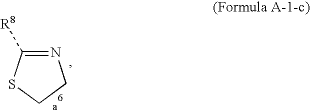
wherein Formula A-1-c is attached on the a⁶ side to adjacent carbons on the d¹ or d² side of Formula A-1;
wherein R⁸ is selected from the group consisting of hydrogen, alkyl, or L₄; 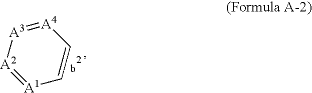
wherein Formula A-2 is attached on the b² side of Formula A-2 to the R¹-substituted ring of formula (I), and A¹, A², A³, A⁴ are (i) —N—; or (ii) —C— substituted with H or alkoxy, wherein the alkoxy may be optionally further substituted with alkoxy on a terminal carbon or up to 3 halogen atoms on a terminal carbon; provided that at least one and no more than two of A¹, A², A³, A⁴ are —N—; and 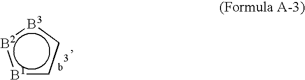
wherein Formula A-3 is attached on the b³ side of Formula A-3 to the R¹-substituted ring of formula (I), and B¹, B² and B³ are independently (i) —CH— optionally substituted with alkyl, aryl, alkoxy, or halogen, (ii) —S—; (iii) —O—; or (iv) —N—; provided that no more than one of B¹, B² or B³ is —S— or —O—, and, provided that when one of B¹, B² or B³ is —S— or —O—, then the adjacent ring members are not —S— or —O—;
s is an integer from 0 to 2;
q is an integer from 0 to 4; provided that when 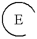
is not substituted with Formulae A-1-a, A-1-b or A-1-c, the sum of q and s is an integer from 0 to 4, and when 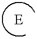
is substituted with one of Formulae A-1-a, A-1-b, or A-1-c, the sum of q and s is an integer from 0 to 2;
R¹ is selected from hydrogen, lower alkyl, —OH, alkoxy, -oxo-NH₂, —NH(alkyl), and —N(alkyl)₂;
R² is selected from the group consisting of hydrogen, alkoxy, alkyl, alkenyl, alkylamino, amino, cyano, dialkylamino, halogen, halogenated alkyl, halogenated alkyloxy, hydroxylated alkyloxy, halogenated —SO₂alkyl, halogenated thioalkyl, hydroxy, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio, thioalkyl, —CO₂H, —C(═O)Oalkyl, —C(═O)NH₂, —C(═O)NH(alkyl), —C(═O)N(alkyl)₂, and L₄;
R³ is independently selected from the group consisting of 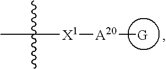
-X-A²⁰-Y¹-A²¹, —CO₂H, —C(═O)Oalkyl, —C(═O)NH₂, —C(═O)NH(alkyl), —C(═O)N(alkyl)₂, and L₄;
wherein X¹ and Y¹ are each independently absent or selected from the group consisting of: -(alkyl)C(═O)N(alkyl)-, —C(═O)N(alkyl)-, —C(═O)NH—, —C(═O)O—, —N(alkyl)-, —N(alkyl)C(═O)—, —N(alkyl)C(═O)NH—, —N(alkyl)C(═O)O—, —N(alkyl)SO₂—, —NH—, —NHC(═O)—, —NHC(═O)N(alkyl)-, —NHC(═O)NH, —NHC(═O)O—, —NHSO₂—, —O—, —OC(═O), —OC(═O)N(alkyl)-, —OC(═O)NH—, —OC(═O)O—, —S—, —SO—, —SO₂—, —SO₂N(alkyl)-, and —SO₂NH—;
A²⁰, when present, is selected from alkyl or alkenyl; and
A²¹ is selected from alkyl, alkenyl, or H;
wherein when A²⁰ or A²¹ is alkyl or alkenyl, the alkyl or alkenyl may be optionally substituted with one or more groups independently selected from: alkoxy, alkylamino, amino, cyano, dialkylamino, halogen, halogenated alkoxy, halogenated —SO₂alkyl, halogenated thioalkyl, hydroxy, —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; 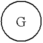
is selected from the group consisting of aryl, cycloalkyl, partially unsaturated carbocycle, heteroaryl, heterocycloalkyl, nine to ten membered benzo-fused cycloalkyl, and nine to ten membered benzo-fused heterocycloalkyl; wherein, the aryl, cycloalkyl, partially unsaturated carbocycle, heteroaryl, heterocycloalkyl, benzo-fused cycloalkyl, or benzo-fused heterocycloalkyl, are optionally substituted with one or more substituents independently selected from halogen, hydroxy, amino, thio, nitro, cyano, alkyl, halogenated alkyl, alkoxy, halogenated alkoxy, alkylamino, —NHC(═O)alkyl, —N(alkyl)C(═O)alkyl, or dialkylamino, —NHC(═O)NH₂, —NHC(═O)NHalkyl, —N(alkyl)C(═O)NHalkyl, —OC(═O)NHalkyl, —NHC(═O)Oalkyl, —N(alkyl)C(═O)Oalkyl, —NHSO₂alkyl, —N(alkyl)SO₂alkyl, thioalkyl, halogenated thioalkyl, —SO₂alkyl, halogenated —SO₂alkyl, —NHC(═O)N(alkyl)₂, —N(alkyl)C(═O)N(alkyl)₂ or —OC(═O)N(alkyl)₂;
L₄ is selected from -M-K-J¹-Matrix or -M-K-J³-X-Matrix;
wherein:
M is selected from —C(═O)NH—, —(C═O)O—, —NHC(═O)—, —NH(C═O)NH—, NH(C═O)O—, —O(C═O)NH—, —OC(═O)O—, —O(C═O)—, —O—, —NH—, —(CH₂)₁₋₃O—, —SS—, or —S—;
K is selected from the group consisting of —(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)NH(CH₂)ᵣ₂]ₙ(CH₂)ₚ—
—(CH₂)ₘNH(C═O)(CH₂)ₙNH(C═O)(CH₂)ₚ—,
—(CH₂)ₘC(═O)NH(CH₂)ₙNH(CH₂)ₚ—,
—(CH₂)ₘC(═O)NH(CH₂)ₙNHC(═O)(CH₂)ₚ—,
—(CH₂)ₘNH(C═O)(CH₂CH₂(OCH₂CH₂)ₙNH(C═O)(CH₂)ₚ—,
—(CH₂)ₘ(C═O)NHCH₂CH₂(OCH₂CH₂)ₙ(C═O)NHCH₂(CH₂)ₚ—,
—(CH₂)ₘ(C═O)NHCH₂(CH₂)ₙ(C═O)NHCH₂(CH₂)ₚ—,
—(CH₂)ₘNH(CH₂)ₜ—, (CH₂)ₘO(CH₂)ₜ—, —(CH₂)ₘS(CH₂)ₜ—,
—(CH₂)ₘS(═O)(CH₂)ₜ—, —(CH₂)ₘSO₂(CH₂)ₜ—,
—(CH₂)ₘNH(C═O)(CH₂)ₜ—,
—(CH₂)ₘ(C═O)NH(CH₂)ₜ—,
—(CH₂)ₘNH(C═O)NH(CH₂)ₜ—,
—(CH₂)ₘSS(CH₂)ₜ—,
—(CH₂)ₘ(SCH₂CH₂)ₙ(CH₂)ₚ—,
—(CH₂)ₘNH(C═O)CH₂CH₂(SCH₂CH₂)ₙNH(C═O)(CH₂)ₚ—,
—(CH₂)ₘ(C═O)NHCH₂CH₂(SCH₂CH₂)ₙ(C═O)NHCH₂(CH₂)ₚ—,
—(CH₂)ms(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—, and
—(CH₂)ₘ(OCH₂CH₂)ₙSS(CH₂)ₚ—;
m is 1 to 7;
n is 1 to 10;
p is 0 to 7;
r and r2 are 1 to 5;
t is 1 to 7;
J¹ is selected from —C(═O)NH—, —(C═O)O—, —NHC(═O)—, —NH(C═O)NH—, NH(C═O)O—, —NH—, —O(C═O)NH—, —OC(═O)O—, —O(C═O)—, —O—, —(CH₂)₁₋₃O—, —SS—, —S—, —SS—, —SCH₂(CHOH)—, —CH═NNH—, 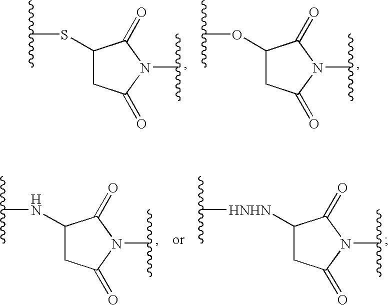
J³ is selected from the group consisting of —NH—, —O—, —NHNH—, —NHNH═CH—, —NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —CH═NNH—, 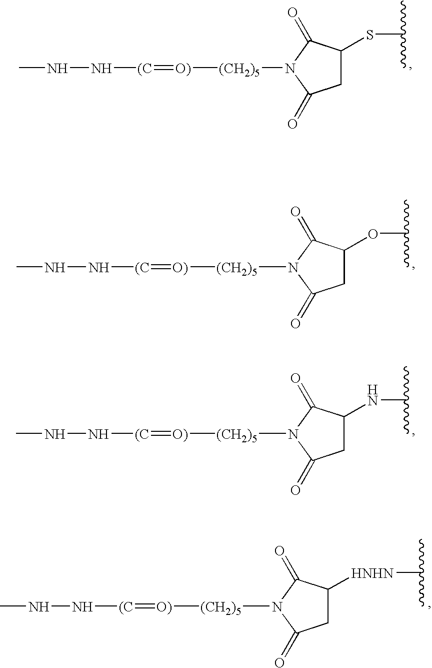
—NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHNH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C═O)NHNH(C═O)NH—, and —NH(CH₂)₆—NH(C═O)CH₂CH₂SS—;
provided that in L₄ the attachment of M to K to J¹ or J³ is not through a N—O or O—N bond, a N—S or S—N bond, an O—S or S—O bond, or an O—O bond;
X is selected from biotin/avidin, biotin/streptavidin, iminobiotin/avidin, or iminobiotin/streptavidin, biotin/NeutrAvidin™, or iminobiotin/NeutrAvidin™;
R⁴ is a substituent independently selected from:
(a)
(b) 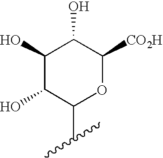
provided that R⁶ is not 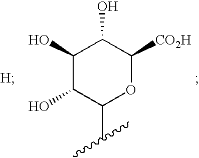
(c) —CH₂— or C₁₋₆alkenyl substituted with one group selected from: —H, -methyl, —Oalkyl, —CH₂OH, —CH(CH₃)OH, cyano, halogen, amino, —(CH₂)₁₋₄alkylamino, —(CH₂)₁₋₄dialkylamino, —O(C═O)alkyl, —(C═O)OH, —C(═O)Oalkyl, —C(═O)Oaryl, —C(═O)Oheteroaryl, —(C═O)NH₂, —(C═O)NHalkyl, —(C═O)N(alkyl)₂, —C(═O)alkyl, -phenyl-OCH₃ or -phenyl-OC(═O)alkyl;
(d) —C(═O)(CH₂CH₂O—)₁₋₁₀ terminating with H, methyl, ethyl, or benzyl;
(e) —C(═O)CH₂O(CH₂CH₂O—)₁₋₁₀ terminating with H, methyl, ethyl, or benzyl;
(f) —C(═O)alkyl, or —C(═O)(C₃₋₆)cycloalkyl, wherein said —C(═O)alkyl, and —C(═O)(C₃₋₆)cycloalkyl may be optionally substituted with one or more groups independently selected from: —OH, —Oalkyl, —Oalkylaryl, —NH₂, —NHalkyl, —N(alkyl)₂, heterocycloalkyl, —NHC(═O)alkyl, —NHSO₂alkyl, or —OC(═O)alkyl;
(g) —C(═O)(CH₂)₁₋₃aryl, —C(═O)aryl, —C(═O)(CH₂)₁₋₃heteroaryl, or —C(═O)heteroaryl, wherein said —C(═O)(CH₂)₁₋₃aryl, —C(═O)aryl, —C(═O)(CH₂)₁₋₃heteroaryl, and —C(═O)heteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(h) —C(═O)(CH₂)₁₋₆C(═O)— terminating with methyl, ethyl, —OH, —Oalkyl, —NH₂, —NHalkyl, —N(alkyl)₂, or heterocycloalkyl;
(hh) —C(═O)alkylOC(═O)alkyl- terminating with —OH, —Oalkyl, —NH₂, —NHalkyl, —N(alkyl)₂, or heterocycloalkyl;
(i) —C(═O)O(CH₂CH₂O—)₁₋₁₀ terminating with H, methyl, ethyl, or benzyl;
(j) —C(═O)Oalkyl, or —C(═O)O(C₃₋₆)cycloalkyl, wherein said —C(═O)Oalkyl, and —C(═O)O(C₃₋₆)cycloalkyl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —C(═O)OH, —C(═O)Oalkyl, —C(═O)NH₂, —C(═O)NHalkyl, or —C(═O)N(alkyl)₂;
(k) —C(═O)O(CH₂)₁₋₃aryl, —C(═O)Oaryl, —C(═O)O(CH₂)₁₋₃heteroaryl, or —C(═O)Oheteroaryl, wherein said —C(═O)O(CH₂)₁₋₃aryl, —C(═O)Oaryl, —C(═O)O(CH₂)₁₋₃heteroaryl, or —C(═O)Oheteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(l) —C(═O)NH(CH₂CH₂O—)₁₋₁₀ terminating with —H, methyl, ethyl, benzyl, —CH₂CH₂NH₂, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl or —C(═O)alkyl;
(m) —C(═O)NH₂, —C(═O)NH(C₁₋₂₀)alkyl, —C(═O)NH(C₃₋₆)cycloalkyl, or —C(═O)N(alkyl)₂, wherein said —C(═O)NH(C₁₋₂₀)alkyl, —C(═O)NH(C₃₋₆)cycloalkyl, and —C(═O)N(alkyl)₂ may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, —OC(═O)alkyl, —OC(═O)alkenyl, —NHC(═O)aryl, —C(═O)OH, —C(═O)Oalkyl, —C(═O)NH₂, —C(═O)NHalkyl, or —C(═O)N(alkyl)₂; and, wherein the aryl portion of said —NHC(═O)aryl may be optionally substituted with one or more groups independently selected from: alkyl, —OH, —Oalkyl, —NH₂, —NH alkyl, —N(alkyl)₂, halogen or nitrile;
(n) —C(═O)NH(CH₂)₁₋₃aryl, —C(═O)NHaryl, —C(═O)NH(CH₂)₁₋₃heteroaryl, or —C(═O)NHheteroaryl, wherein said —C(═O)NH(CH₂)₁₋₃aryl, —C(═O)NHaryl, —C(═O)NH(CH₂)₁₋₃heteroaryl, and —C(═O)NHheteroaryl may be optionally substituted with one or more groups independently selected from: —OH, —Oalkyl, —NH₂, —NHalkyl, —N(alkyl)₂, heterocycloalkyl, —NHC(═O)alkyl, —NHSO₂alkyl, halogen, nitrile, or —OC(═O)alkyl;
(o)—C(═O)NHCH₂CH₂NH(CH₂CH₂NH—)₀₋₃ terminating with H, methyl, ethyl, —CH₂CH₂NHalkyl, —CH₂CH₂N(alkyl)₂, —CH₂CH₂-1-pyrrolidinyl, —CH₂CH₂-1-piperidinyl, —CH₂CH₂-4-morpholinyl, —CH₂CH₂-1-piperazinyl, —CH₂CH₂-1-(4-CH₃)-piperazinyl, —CH₂CH₂OH, —CH₂CH₂OCH₃, —CH₂CH₂OCH₂CH₃, —CH₂CH₂C(═O)alkyl, or —C(═O)aryl; wherein the aryl portion of said —C(═O)aryl may be optionally substituted with one or more groups independently selected from: alkyl, —OH, —Oalkyl, —NH₂, —NHalkyl, —N(alkyl)₂, halogen or nitrile;
(p) —C(═S)NH₂;
(q) —C(═S)NHalkyl;
(r) —C(═S)N(alkyl)₂;
(s) —SO₂NH₂;
(t) —SO₂NHalkyl;
(u) —SO₂N(alkyl)₂;
(v) —P(═O)(OCH₃)₂;
(w) —P(═O)(OCH₂CH₃)₂; or
(x) L₅
L₅ is selected from -M¹-K-J¹-Matrix or -M¹-K-J³-X-Matrix wherein:
M¹ is selected from —C(═O)—, —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄₀(C═O)NH—, —(CH₂)₂₋₄OC(═O)—, —(CH₂)₂₋₄(C═O)O—, —(CH₂)₂₋₄S—, —(CH₂)₂₋₄SS—, or —(CH₂)₂₋₄O—;
provided that in L₅ the attachment of M¹ to K to J¹ or J³ is not through a N—O or O—N bond, a N—S or S—N bond, an O—S or S—O bond, or an O—O bond;
R⁶ is selected from the group consisting of H and 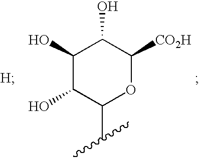
provided that if R⁴ is 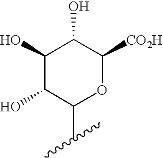
then R⁶ is H;
L₃ is absent or is a linking group selected from the group consisting of alkylidyl, carbonyl, -alkylidyl-C(═O)—, —C(═O)-alkylidyl-, -alkylidylC(═O)alkylidyl-, or —SO₂—;
B is selected from the group consisting of an aryl, a heteroaryl, a nine to ten membered benzo-fused cycloalkyl, a nine to ten membered benzo-fused heterocycloalkyl, —CH(R⁹)aryl, and —CH(R⁹)heteroaryl; wherein the aromatic portion of said B is optionally substituted with R⁵; and wherein R⁹ is a substituent selected from hydrogen, alkyl, or cycloalkyl, wherein said alkyl is optionally substituted with alkylamino, amino, cyano, dialkylamino, halogenated alkyl, halogenated alkyloxy, —SO₂alkyl, or hydroxy;
R⁵ is independently selected from the group consisting of L₄, alkyl; alkylamino, alkyloxy, amino, —C(═O)NH₂, —C(═O)NH(alkyl), —C(═O)N(dialkyl), —C(═O)Oalkyl, —C(═O)OH, —CH₂OH, cyano, dialkylamino, halogen, halogenated alkyl, halogenated alkyloxy, halogenated SO₂-alkyl, halogenated thioalkyl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, —SO₂NH₂, thio, thioalkyl, 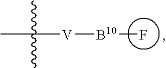
and -V-B¹⁰-W-B²⁰; wherein,
V and W are each independently absent or selected from the group consisting of: —C(═O), —C(═O)N(alkyl)-, —C(═O)NH—, —C(═O)O—, —N(alkyl)-, —N(alkyl)C(═O)—, —N(alkyl)C(═O)N(alkyl)-, —N(alkyl)C(═O)NH—, —N(alkyl)C(═O)O—, —N(alkyl)SO₂—, —NH—, —NHC(═O)—, —NHC(═O)N(alkyl)-, —NHC(═O)NH—, —NHC(═O)O—, —NHSO₂—, —O—, —OC(═O), —OC(═O)N(alkyl)-, —OC(═O)NH—, —OC(═O)O—, —S—, —SO—, —SO₂—, —SO₂N(alkyl)- and —SO₂NH—;
B¹⁰ is absent or selected from alkyl or alkenyl;
B²⁰ is absent or selected from alkyl, alkenyl, or H;
wherein, when B¹⁰ or B²⁰ is alkyl or alkenyl, the alkyl or alkenyl group may be optionally substituted with one or more groups independently selected from:
alkoxy, alkylamino, amino, cyano, dialkylamino, halogen, halogenated alkoxy, halogenated —SO₂alkyl, halogenated thioalkyl, hydroxy —N(alkyl)C(═O)alkyl, —N(alkyl)C(═O)N(alkyl)₂, —N(alkyl)C(═O)NH₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, —OC(═O)alkyl, —OC(═O)N(alkyl)₂, —OC(═O)NHalkyl, —OC(═O)Oalkyl, —SO₂alkyl, thio or thioalkyl; and 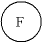
is selected from the group consisting of: an aryl, a cycloalkyl, a partially unsaturated carbocycle, a heteroaryl, a heterocycloalkyl, a nine to ten membered benzo-fused cycloalkyl, and a nine to ten membered benzo-fused heterocycloalkyl,
wherein, the aryl, cycloalkyl, partially unsaturated carbocycle, heteroaryl, heterocycloalkyl, benzo-fused cycloalkyl, or benzo-fused heterocycloalkyl, is optionally substituted with one or more substituents independently selected from: alkoxy, alkylamino, amino, cyano, dialkylamino, halogen, halogenated alkoxy, halogenated alkyl, halogenated —SO₂alkyl, halogenated thioalkyl, heteroaryl, hydroxy, hydroxy alkyl, —N(alkyl)C(═O)alkyl, —N(alkyl)C(═O)N(alkyl)₂, —N(alkyl)C(═O)NHalkyl, —N(alkyl)C(═O)Oalkyl, —N(alkyl)SO₂alkyl, —NHC(═O)alkyl, —NHC(═O)N(alkyl)₂, —NHC(═O)NH₂, —NHC(═O)NHalkyl, —NHC(═O)Oalkyl, —NHSO₂alkyl, nitro, —OC(═O)N(alkyl)₂, —OC(═O)NHalkyl, —SO₂alkyl, thio or thioalkyl;
provided that at least one, and no more than one, L₄ or L₅ is present.

2. The compound of claim 1, wherein 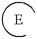
is: 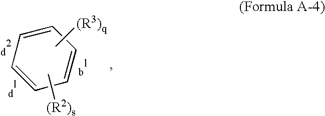
wherein Formula A-4 is attached on the b¹ side of Formula A-4 to the R¹ substituted ring of formulae (I); and wherein
q is an integer from 0 to 4; provided that when 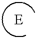
is Formula A-4, the sum of q and s is an integer from 0 to 4.

3. The compound of claim 1, wherein L₄ is selected from -M-K-J¹-Matrix or -M-K-J³-X-Matrix; wherein M is selected from —C(═O)NH—, —O—, —NHC(═O)O—, —S—, or —SS—; and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ[NH(C═O)(CH₂)ᵣ]ₙ(CH₂)ₚ—,
—(CH₂)ₘ[(C═O)NH(CH₂)ᵣ₂]ₙ(CH₂)ₚ—,
—(CH₂)ₘNH(C═O)(CH₂)ₙNH(C═O)(CH₂)ₚ—,
—(CH₂)ₙC(═O)NH(CH₂)ₙNH(CH₂)ₚ—,
—(CH₂)ₘC(═O)NH(CH₂)ₙNHC(═O)(CH₂)ₚ—,
—(CH₂)ₘS(CH₂)ₙO(CH₂)ₚ—
—(CH₂CH₂SCH₂CH₂O)ₙ(CH₂)ₚ—
—(CH₂)ₘS(CH₂)C(═O)NH(CH₂)ₙ—
—(CH₂)ₘSS(CH₂)ₙC(═O)NH(CH₂)ₚ—
—(CH₂)ₘNHC(═O)(CH₂)ₙSS(CH₂)ₚ—
—(CH₂)ₘNH(C═O)CH₂OCH₂C(═O)NH(CH₂)ₙ(OCH₂CH₂)ₚCH₂)—
—(CH₂)ₘ(OCH₂CH₂)ₙC(═O)NH(CH₂)ₚ—
—(CH₂)ₘC(═O)NH(CH₂)ₙ(OCH₂CH₂)ₚ—,
—(CH₂)ₘ(OCH₂CH₂)ₙNHC(═O)(CH₂)ₚ—,
—(CH₂)ₘ(OCH₂CH₂)ₙSS(CH₂)ₚ—; and
wherein J¹ is selected from —C(═O)NH—, —(C═O)O—, —NHC(═O)—, —NH(C═O)NH—, —NH(C═O)O—, —O(C═O)NH—, —OC(═O)O—, —O(C═O)—, —O—, —NH—, —(CH₂)₁₋₃O—, —SS—, —S—, —SCH₂(CHOH)—, —CH═NNH—, 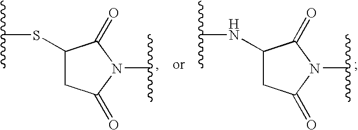
and
wherein J³ is selected from the group consisting of —NH—, —O—, —NHNH—,
—NHNH═CH—, —NHNH—(C═O)—, —NHNH—(C═O)O—, —NHNH—(C═O)NH—, —NH—(CH₂)₆—NH—(C═O)CH₂—S—, —NH—(CH₂)₆—NH—(C═O)CH₂O—, —NH—(CH₂)₆—NH—(C═O)CH₂—NH—, —NH—(CH₂)₅(C═O)—NH—(CH₂)₅(C═O)—NHN═CH—, —NH—(CH₂)₅(C═O)—NH—(CH₂)₅(C═O)—NHNH—(C═O)—, —NH—(CH₂)₅(C═O)—NH—(CH₂)₅(C═O)—NHNH—(C═O)O—, —NH—(CH₂)₅(C═O)—NH—(CH₂)₅(C═O)—NHNH—(C═O)NH—, —NH—(CH₂)₅(C═O)—NH—, —NH—(CH₂)₅(C═O)—O—, —NH—(CH₂)₅(C═O)—NHNH—, —NH—(CH₂)₅(C═O)—NHN═CH—, —NH—(CH₂)₅(C═O)—NHNH(C═O)—, —NH—(CH₂)₅(C═O)—NHNH(C═O)O, —NH—(CH₂)₅(C═O)—NHNH(C═O)NH—, —NH—(CH₂)₂—SS(CH₂)₂(C═O)NH—, —NH—(CH₂)₂—SS—(CH₂)₂(C═O)O—, —NH—(CH₂)₂—SS—(CH₂)₂(C═O)NHNH—, —NH—(CH₂)₂—SS—(CH₂)₂(C═O)NHN═CH—, —NH—(CH₂)₂—SS—(CH₂)₂(C═O)NHNH(C═O)—, —NH—(CH₂)₂—SS—(CH₂)₂(C═O)NHNH(C═O)O—, —NH—(CH₂)₂—SS—(CH₂)₂(C═O)NHNH(C═O)NH—, and —NH(CH₂)₆—NH(C═O)CH₂CH₂SS—;
provided that in L₄ the attachment of M to K to J¹ or J³ is not through a N—O or O—N bond, a N—S or S—N bond, an O—S or S—O bond, or an O—O bond.

4. The compound of claim 1, wherein L₄ is selected from -M-K-J¹-Matrix or -M-K-J³-X-Matrix; wherein M is selected from —C(═O)NH— or —O—; and wherein K is selected from
—(CH₂)ₘ(CH₂)ₙ(CH₂)ₚ—,
—(CH₂)ₘ(OCH₂CH₂)ₙ(CH₂)ₚ—,
—(CH₂)ₘ(OCH₂CH₂)ₙ(OCH₂)ₚ—,
—(CH₂)ₘ(OCH₂CH₂)ₙNHC(═O)(CH₂)ₚ—,
—(CH₂)ₘ(OCH₂CH₂)ₙSS(CH₂)ₚ—; and
wherein J¹ is selected from —NH—, —NHC(═O)O—, —OC(═O)—, —C(═O)O—, —C(—O)NH—, or —NHC(═O)—, —CH═NNH—, 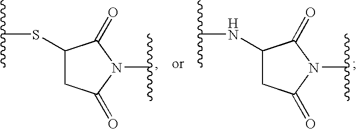
and
J³ is selected from —NH—, —NHC(═O)O—, —OC(═O)—, —C(═O)O—, —C(═O)NH—, or —NHC(═O)—;
provided that in L₄ the attachment of M to K to J¹ or J³ is not through a N—O or O—N bond, a N—S or S—N bond, an O—S or S—O bond, or an O—O bond.

5. The compound of claim 1 and wherein L₅ is selected from -M¹-K-J¹-Matrix or -M¹-K-J³-X-Matrix; wherein: M¹ is selected from —C(═O)NH—, —(CH₂)₂₋₄NHC(═O)—, —(CH₂)₂₋₄NH(C═O)NH—, —(CH₂)₂₋₄(C═O)NH—, —(CH₂)₂₋₄NHC(═O)O—, —(CH₂)₂₋₄(C═O)O—, —(CH₂)₂₋₄S—, or —(CH₂)₂₋₄O—; provided that in L₅ the attachment of M¹ to K to J¹ or J³ is not through a N—O or O—N bond, a N—S or S—N bond, an O—S or S—O bond, or an O—O bond.

6. The compound of claim 1, wherein L₄ is -M-K-J¹-Matrix or -M-K-J³-X-Matrix and L₅ is -M¹-K-J¹-Matrix or -M¹-K-J³-X-Matrix and L₄ and L₅ are of a length and degree of flexibility that permits binding between the precursor compound and the target proteins.

7. The compound of claim 1, wherein said Matrix comprises a solid support material.

8. The compound of claim 7, wherein said solid support material is selected from the group consisting of gel, cellulose, glass, plastic material, beads, and plates.

9. A compound of claim 1 wherein: Matrix is selected from the group consisting of Ciphergen PS10 chip; Ciphergen PS20 chip; Reacti-Gel; UltraLink; UltraLink DADPA, PharmaLink, AminoLink; CarboLink; SulfoLink; MagnaBind bead; and UltraLink maleimide.

10. A method of identifying a biological molecule that binds to a compound of claim 1, comprising the steps of:
(1) contacting a test sample with the compound of claim 1 under a condition that allows a biological molecule within the test sample to bind to the compound, wherein said biological molecule is immobilized to a matrix via binding to the compound;
(2) releasing the bound biological molecule from the matrix; and
(3) characterizing the released biological molecule.

11. The method of claim 10, further comprising a step of washing the matrix with a suitable buffer to remove non-specifically bound artifacts after the contacting step.

12. The method of claim 10, wherein the biological molecule that binds to the compound of claim 1 comprises a polypeptide, a polynucleotide, a carbohydrate, or a lipid.

13. The method of claim 12, wherein the biological molecule that binds to the compound of claim 1 comprises a polypeptide.

14. The method of claim 10, wherein the biological molecule binds to the compound of claim 1 covalently.

15. The method of claim 10, wherein the biological molecule binds to the compound of claim 1 non-covalently.

16. The method of claim 10, wherein the releasing step comprises the step of contacting the matrix with excess amount of a core structure of the compound of claim 1.

17. The method of claim 10, wherein the releasing step comprises the step of breaking a disulfide bond linkage contained within the compound of claim 1.

18. The method of claim 10, wherein the releasing step comprises the step of contacting the matrix with an excess amount of biotin when the compound of claim 1 contains a specific binding pair of biotin and its complementary protein.

19. The method of claim 10, wherein the matrix comprises a protein-chip.

20. The method of claim 19, wherein the releasing step comprises the steps of:
(1) adding an energy-absorbing molecule to the surface of the protein-chip; and
(2) exposing the surface of the protein-chip to a nitrogen laser.

21. The method of claim 10, further comprising a step of comparing the biological activity of the biological molecule in the presence and absence of the compound of claim 1, a precursor compound to the compound of claim 1, or a core structure of the compound of claim 1.

22. A method of identifying a biological molecule that binds to a precursor compound to a compound of claim 1, comprising the steps of:
(1) contacting a test sample with the precursor compound under a condition that allows a biological molecule within the test sample to bind to the precursor compound;
(2) immobilizing the precursor compound to a matrix to form the compound of claim 1, wherein said biological molecule is immobilized to the matrix via binding to the compound of claim 1;
(3) releasing the bound biological molecule from the matrix; and
(4) characterizing the released biological molecule.

23. The method of claim 22, further comprising a step of washing the matrix with a suitable buffer to remove non-specifically bound artifacts after the contacting step.

24. The method of claim 22, wherein the biological molecule that binds to the compound of claim 1 comprises a polypeptide, a polynucleotides, a carbohydrate, or a lipid.

25. The method of claim 24, wherein the biological molecule that binds to the compound of claim 1 comprises a polypeptide.

26. The method of claim 22, wherein the biological molecule binds to the compound of claim 1 covalently.

27. The method of claim 22, wherein the biological molecule binds to the compound of claim 1 non-covalently.

28. The method of claim 22, wherein the releasing step comprises the step of contacting the matrix with excess amount of a core structure of the compound of claim 1.

29. The method of claim 22, wherein the releasing step comprises the step of breaking a disulfide bond linkage contained within the compound of claim 1.

30. The method of claim 22, wherein the releasing step comprises the step of contacting the matrix with excess amount of biotin when the compound of claim 1 contains a specific binding pair of biotin and its complementary protein.

31. The method of claim 22, wherein the matrix comprises a protein-chip.

32. The method of claim 31, wherein the releasing step comprises the steps of:
(1) adding an energy-absorbing molecule to the surface of the protein-chip; and
(2) exposing the surface of the protein-chip to a nitrogen laser.

33. The method of claim 22, further comprising a step of comparing the biological activity of the biological molecule in the presence and absence of the compound of claim 1, the precursor compound to the compound of claim 1, or a core structure of the compound of claim 1.

34. A method of identifying a compound that binds to tubulin, comprising the steps of:
(a) synthesizing a compound that mimics the core structure of a compound of claim 1; and
(b) determining the ability of the compound that mimics the core structure of the compound of claim 1 to bind to polymerizing tubulin.

35. The method of claim 34, wherein in the step of synthesizing, the compound of claim 1 is:

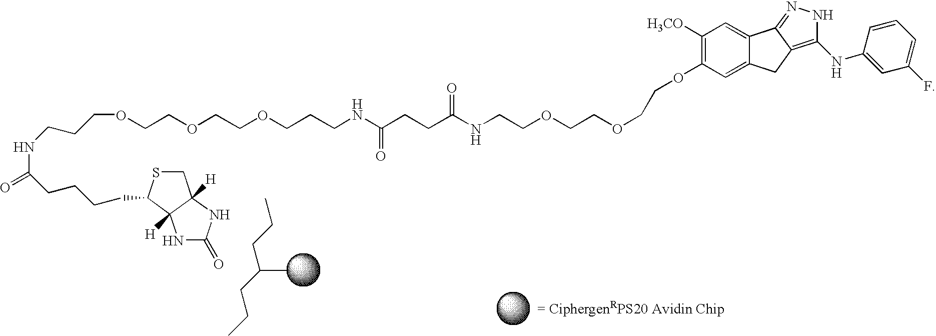

36. A method of regulating an activity of polymerizing tubulin, comprising a step of contacting the polymerizing tubulin with a compound of claim 1, a precursor compound to a compound of claim 1, or a core structure of a compound of claim 1.

37. The method of claim 36, comprising a step of contacting the polymerizing tubulin with: 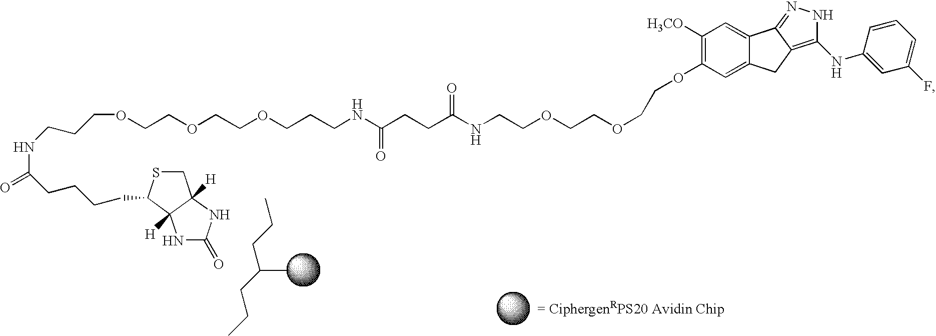
a precursor compound or a core structure compound thereof.

38. A method of disrupting the function of the mitotic spindle in a cell comprising the step of contacting the cell with a compound of claim 1, a precursor compound to a compound of claim 1, or a core structure of a compound of claim 1.

39. The method of claim 38, comprising the step of contacting the cell with: 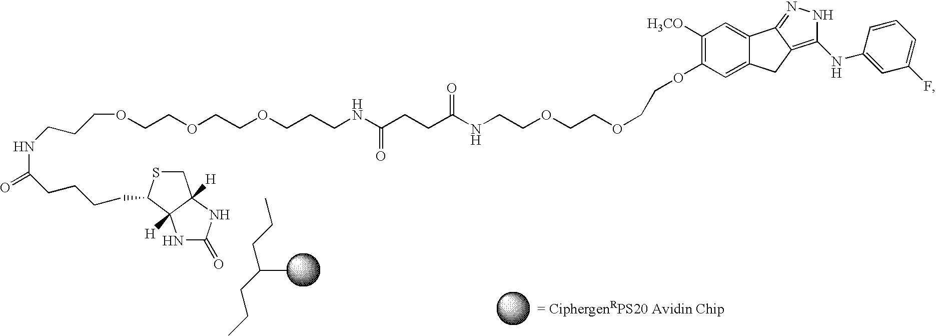
a precursor compound or a core structure compound thereof.
